(19) United States
(12) Patent Application Publication
(10) Pub. No.: US 2026/0167621 A1
(43) Pub. Date: Jun. 18, 2026

(54) Three Cluster Galactose Type Compound, Conjugate, Making Method and Use Thereof

(71) Applicant: GUANGZHOU RIBOBIO CO., LTD., Guangzhou, Guangdong (CN)

(72) Inventors: Bill Biliang ZHANG, Boston, MA (US); Haoting ZHAO, Guangzhou, Guangdong (CN); Naixing WU, Guangzhou, Guangdong (CN)

(21) Appl. No.: 18/845,843

(22) PCT Filed: Mar. 26, 2024
(86) PCT No.: PCT/CN2024/083850

§ 371 Date: Sep. 10, 2024

(30) Foreign Application Priority Data
Jul. 20, 2023 (CN) 202310897323.4

(51) Int. Cl. C07D 401/14; C12N 15/113

(52) U.S. Cl. CPC C07D 401/14; C12N 15/113

(57) ABSTRACT

This disclosure relates to a compound of formula (I), and a conjugate linked by this compound and pharmaceutical molecule. This disclosure also relates the making method of this compound, as well as the use of the said compound and the conjugate in making pharmaceutical related to cure or prevent the disease related to expression or over expression of gene in hepatocyte.

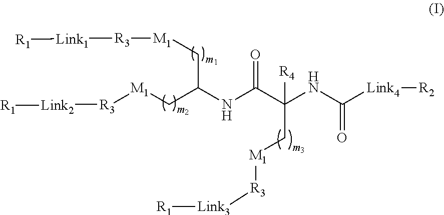

### CROSS-REFERENCE TO RELATED APPLICATIONS

The present application is a National Stage of International Patent Application No. PCT/CN2024/083850 filed on Mar. 26, 2024, which claims the benefit of priority from Chinese Application No. 202310897323.4, filed on Jul. 20, 2023, the disclosure of which is incorporated herein by reference in its entirety.

### SEQUENCE LISTING

The instant disclosure contains a Sequence Listing which has been submitted electronically in ASCII format and is hereby incorporated by reference in its entirety. Said ASCII copy is named PN242758.xml and is 17,704 bytes in size. The date of creation is 2024-06-14.

### TECHNICAL FIELD

This disclosure relates to biology technology field, specifically relates to gel formulations of three cluster galactose type compound and conjugate. This disclosure also relates the making method of the said compound, and the use of the said compound and the conjugate in making pharmaceutical related to cure or prevent the disease related to the expression or over expression of gene in hepatocyte.

### BACKGROUND

Asialoglycoprotein receptor (ASGPR) is an abundant endocytic receptors for heterologous oligomers, which mainly exists on the surface of hepatic parenchymal cell membrane facing into the side of antroid space, is specifically recognized by sugar, since the exposed minor end of each glycoprotein is the residue of galactose after removing terminal sialic acid by enzymatic hydrolysis or acidolysis, the sugar binding specificity of ASGPR is actually originated from galactosyl, which is also named galactose-specific receptor. ASGPR mainly distributes in hepatic parenchymal cell and low content in other cells, so it becomes the best receptor of hepatic targeting transport.

The glycoprotein with residue end of non-reducing galactose (Gal) or N-acetylgalactosamine (GalNAc) of can be recognized by ASGPR, the binding affinity between GalNAc and ASGPR is 50 times higher than Gal (Iobst S T et al, J Biol Chem, 1996, 271 (12): 6686-6693). In vitro experiment showed that the clustered sugar residues can occupy the binding site of receptor at the same time to make its binding affinity much higher than unclustered sugar residues.

The hepatic targeting oligonucleotide mediated by ASGPR receptor is new breakthrough in the nucleic acid innovation pharmaceutical researching field. GalNAc (N-acetylgalactosamine) conjugated modification is the most common used small nucleic acid pharmaceutical delivery system currently. Nowadays, most conjugate structures about GalNAc are tri-antennary GalNAc structure, specifically means that the GalNAc trivalently conjugates to 3′ or 5′ end of sense strand of different siRNA sequences to form polysaccharide-siRNA monoconjugate, thereby accomplishing Hepatocyte targeting delivery and making pharmaceutical entry into cell by endocytosis to play its role.

In 2012, the US Alnylam Pharmaceuticals, Inc. used tri-antennary GalNAc structure invented by predecessor to covalently bind to small interference RNA (siRNA), thereby accomplishing in vivo Hepatocyte targeting delivery. By applying this technology, researchers developed pharmaceuticals related to amyloidosis, hemophilia, hypercholesterolemia, hepatic porphyrias, hepatitis B, etc. In 2014, the US Ionis Pharmaceuticals, Inc. used tri-antennary GalNAc to covalently bind to antisense nucleic acid, thereby accomplishing animal in vivo hepatic targeting administration, the activity of bound antisense nucleic acid improved 10 times (Prakash T P et al, Nucleic Acids Res. 42, 8796-807).

However, the hepatic targeting oligonucleotides technology mediated by ASGPR receptor is still needed to be further developed and improved, people still study on other structures different with the traditional tri-antennary GalNAc structure.

### SUMMARY

This disclosure is aim at solving the above mentioned questions, providing a three cluster galactose type compound, different with traditional tri-antennary GalNAc structure, which can remain good hepatic targeting binding affinity, good hepatic targeting, better hydrophilicity, low toxicity, as well as easy to synthesized.

For developing three cluster galactose type compound with new structure, inventors intend to make use of multiple amino acids to synthesize compounds with different branch structure, such as glutamic acid, aspartate or lysine and so on, but these structures are not obvious in improving the binding affinity to ASGPR receptor. After continuous trail and research, inventors found that using structure condensed by L-serine and serinol as branch structure can obtain three cluster galactose type compound with better hydrophilicity and low toxicity, which have very powerful application value in clinic.

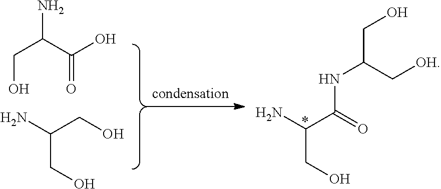

In one aspect, this disclosure provides a compound of formula (I), or a pharmaceutical acceptable salt, tautomer or stereoisomer thereof:

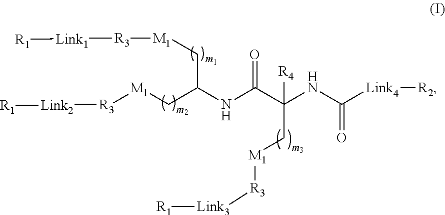

wherein each groups are defined as below.

In other aspect, this disclosure provides a conjugate with a structure shown in formula (III):

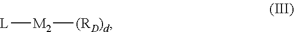

wherein each groups are defined as below.

In other aspect, this disclosure provides a pharmaceutical composition which includes conjugates of this disclosure, and optional selected pharmaceutical acceptable carrier, excipient, adjuvant or vehicle, and optionally other therapeutic agents.

In other aspect, this disclosure provides use of conjugate of this disclosure in making pharmaceutical related to cure or prevent the disease related to expression or over expression of gene in hepatocyte.

In other aspect, this disclosure provides method of curing or preventing the disease related to expression or over expression of gene in hepatocyte in subject, including administrating conjugate of this disclosure or pharmaceutical composition of this disclosure to subject.

In other aspect, this disclosure provides conjugate of this disclosure or pharmaceutical composition of this disclosure, which are used in curing or preventing the disease related to expression or over expression of gene in hepatocyte.

In specific examples, the said gene is selected from at least one of HBV gene group, HCV gene group, PCSK9, xanthine oxidase, URAT1, APOB, hepatic fibrosis related gene (AP3S2, AQP2, AZINI, DEGSI, STXBP5L, TLR4, TRPM5), non-alcoholic fatty liver disease related gene (PNPLA3, FDFTI), or primary biliary cirrhosis related gene (HLA-DQB1, IL-12, IL-12RB2), or the combination thereof.

In other specific example, this disclosure is used to cure or prevent the following diseases: hereditary angioedema, familial tyrosinemia type I or type II, Alagille syndrome, α-1-antitrypsindeficiency, bile acid synthesis and metabolism defect, biliary atresia, cystic fibrosis liver disease, idiopathic neonatal hepatitis, mitochondrial liver disease, progressive familial intrahepatic cholestasis, primary sclerosing cholangitis, transthyretin amyloidosis, hemophilia, homozygous familial hypercholesterolemia, hyperlipidemia, hepatitis B virus (HBV), hepatitis C virus (HCV), steatohepatitis, nonalcoholic steatohepatitis (NASH), non-alcoholic fatty liver disease (NAFLD), hyperglycemia or disease of abnormally increased hepatic glucose production similar to type I and II diabetes, hepatitis and hepatoporphyrin.

### Definition

### Chemical Definition

Definitions of specific functional groups and chemical terms are described in more detail below.

When the numeric range is listed, it includes each value and the sub-range within the said range. For example, “C₁₋₆ alkyl” includes C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅ and C₅₋₆ alkyl.

“C₁₋₁₀ alkyl” refers to a radical of a straight or branched, saturated hydrocarbon group having 1-10 carbon atoms. In some embodiments, C₁₋₆ alkyl, C₁₋₅ alkyl, C₁₋₄ alkyl, C₁₋₃ alkyl and C₁₂ alkyl are preferred. Examples of C₁₋₆ alkyl include methyl (C₁), ethyl (C₂), n-propyl (C₃), iso-propyl (C₃), n-butyl (C₄), tert-butyl (C₄), sec-butyl (C₄), iso-butyl (C₄), n-pentyl (C₅), 3-pentyl (C₅), pentyl (C₅), neopentyl (C₅), 3-methyl-2-butyl (C₅), tert-pentyl (C₅) and n-hexyl (C₆). The term “C₁₋₆ alkyl” also includes heteroalkyl, wherein one or more (e.g., 1, 2, 3 or 4) carbon atoms are substituted with heteroatoms (e.g., oxygen, sulfur, nitrogen, boron, silicon, or phosphorus). Alkyl groups can be optionally substituted with one or more substituents, for example, with 1 to 5 substituents, 1 to 3 substituents or 1 substituent. Conventional abbreviations of alkyl include Me (—CH₃), Et (—CH₂CH₃), Pr (—CH(CH₃)₂), nPr (—CH₂CH₂CH₃), n-Bu(—CH₂CH₂CH₂CH₃) or i-Bu (—CH₂CH(CH₃)₂).

“C₂₋₆ alkenyl” refers to a radical of a straight or branched hydrocarbon group having 2 to 6 carbon atoms and at least one carbon-carbon double bond. In some embodiments, C₂₋₄ alkenyl is preferred. Examples of C₂₋₆ alkenyl include vinyl (C₂), 1-propenyl (C₃), 2-propenyl (C₃), 1-butenyl (C₄), 2-butenyl (C₄), butadienyl (C₄), pentenyl (C₅), pentadienyl (C₅), hexenyl (C₆), etc. The term “C₂₋₆ alkenyl” also includes heteroalkenyl, wherein one or more (e.g., 1, 2, 3 or 4) carbon atoms are replaced by heteroatoms (e.g., oxygen, sulfur, nitrogen, boron, silicon, or phosphorus). The alkenyl groups can be optionally substituted with one or more substituents, for example, with 1 to 5 substituents, 1 to 3 substituents or 1 substituent.

“C₂₋₆ alkynyl” refers to a radical of a straight or branched hydrocarbon group having 2 to 6 carbon atoms, at least one carbon-carbon triple bond and optionally one or more carbon-carbon double bonds. In some embodiments, C₂₋₄ alkynyl is preferred. Examples of C₂₋₆ alkynyl include, but are not limited to, ethynyl (C₂), 1-propynyl (C₃), 2-propynyl (C₃), 1-butynyl (C₄), 2-butynyl (C₄), pentynyl (C₅), hexynyl (C₆), etc. The term “C₂₋₆ alkynyl” also includes heteroalkynyl, wherein one or more (e.g., 1, 2, 3 or 4) carbon atoms are replaced by heteroatoms (e.g., oxygen, sulfur, nitrogen, boron, silicon, or phosphorus). The alkynyl groups can be substituted with one or more substituents, for example, with 1 to 5 substituents, 1 to 3 substituents or 1 substituent.

“C₁₋₂₅ alkylene” refers to a divalent group formed by removing another hydrogen of the C₁₋₂₅ alkyl, and can be a substituted or unsubstituted alkylene. In some embodiments, C₅₋₂₅ alkylene, C₁₋₂₀ alkylene, C₃₋₂₀ alkylene, C₅₋₂₀ alkylene, C₈₋₂₀ alkylene, C₁₋₁₇ alkylene, C₁₋₁₇ alkylene, C₃₋₁₇ alkylene, C₁₋₁₀ alkylene, C₃₋₁₀ alkylene, C₁₋₆ alkylene, C₃₋₆ alkylene, C₄₋₆ alkylene, C₁₋₄ alkylene, C₂₋₄ alkylene and C₁₋₃ alkylene are preferred. In some embodiments, C₆ alkylene and C₄ alkylene are preferred. The unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), and hexylene (—CH₂CH₂CH₂CH₂CH₂CH₂—), etc. Examples of substituted alkylene groups, such as those substituted with one or more alkyl (methyl) groups, include, but are not limited to, substituted methylene (—CH(CH₃)—, —C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, and —CH₂CH₂C(CH₃)₂—), etc. In some embodiments, a straight chain alkylene is preferred.

“C₀₋₆ alkylene” refers to chemical group and the “C₁₋₆ alkylene” as defined above. “C₀₋₄ alkylene” refers to chemical group and the “C₁₋₄ alkylene” as defined above.

“Halo” or “halogen” refers to fluorine (F), chlorine (Cl), bromine (Br) or iodine (I).

Thus, “C₁₋₁₀ haloalkyl” refers to the above “C₁₋₁₀ alkyl”, which is substituted by one or more halogen. In some embodiments, C₁₋₆ haloalkyl, C₁₋₅ haloalkyl, C₁₋₄ haloalkyl and C₁₋₃ haloalkyl are especially preferred, more prefer C₁₋₂ haloalkyl. Exemplary the said haloalkyl groups include, but are not limited to, —CF₃, —CH₂F, —CHF₂, —CHFCH₂F, —CH₂CHF₂, —CF₂CF₃, —CCl₃, —CH₂Cl, —CHCl₂, and 2,2,2-trifluoro-1,1-dimethyl-ethyl, and the like. The haloalkyl can be substituted at any available point of attachment, for example, with 1 to 5 substituents, 1 to 3 substituents or 1 substituent.

“C₃₋₁₀ cycloalkyl” refers to a radical of a non-aromatic cyclic hydrocarbon group having from 3 to 10 ring carbon atoms and zero heteroatoms, which optionally includes 1, 2 or 3 double bonds or triple bonds. In some embodiments, C₅₋₁₀ cycloalkyl, C₃₋₇ cycloalkyl and C₃₋₆ cycloalkyl are especially preferred, more prefer C₅₋₇ cycloalkyl and C₅₋₆ cycloalkyl. The cycloalkyl also includes a ring system in which the cycloalkyl described herein is fused with one or more aryl or heteroaryl groups, wherein the point of attachment is on the cycloalkyl ring, and in such case, the number of carbon atoms continues to represent the number of carbon atoms in the cycloalkyl system. The cycloalkyl further comprises the cycloalkyl described above, in which the substituents on any non-adjacent carbon atoms are connected to form a bridged ring, together forming a polycyclic alkane sharing two or more carbon atoms. The cycloalkyl further comprises the cycloalkyl described above, in which the substituents on the same carbon atom are connected to form a ring, together forming a polycyclic alkane sharing one carbon atom. Exemplary cycloalkyl groups include, but are not limited to, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), cycloheptyl (C₇), cycloheptenyl (C₇), cycloheptadienyl (C₇), and cycloheptatrienyl (C₇), etc. The cycloalkyl can be substituted with one or more substituents, for example, with 1 to 5 substituents, 1 to 3 substituents or 1 substituent.

“3-10 membered heterocyclyl” refers to a saturated or unsaturated radical of 3-10 membered non-aromatic ring system having ring carbon atoms and 1 to 5 ring heteroatoms, wherein each of the heteroatoms is independently selected from at least one of nitrogen, oxygen, sulfur, boron, phosphorus and silicon, optionally wherein 1, 2 or 3 double or triple bonds are contained. In the heterocyclyl containing one or more nitrogen atoms, the point of attachment can be a carbon or nitrogen atom as long as the valence permits. In some embodiments, 5-10 membered heterocyclyl is preferred, which is a radical of 5-10 membered non-aromatic ring system having ring carbon atoms and 1 to 5 ring heteroatoms; in some embodiments, 3-7 membered heterocyclyl is preferred, which is a radical of 3-7 membered non-aromatic ring system having ring carbon atoms and 1 to 4 ring heteroatoms; 5-7 membered heterocyclyl is preferred, which is a radical of 5-7 membered non-aromatic ring system having ring carbon atoms and 1 to 3 ring heteroatoms; 3-6 membered heterocyclyl is preferred, which is a radical of 3-6 membered non-aromatic ring system having ring carbon atoms and 1 to 3 ring heteroatoms; 4-6 membered heterocyclyl is preferred, which is a radical of 4-6 membered non-aromatic ring system having ring carbon atoms and 1 to 3 ring heteroatoms; 5-6 membered heterocyclyl is more preferred, which is a radical of 5-6 membered non-aromatic ring system having ring carbon atoms and 1 to 3 ring heteroatoms. The heterocyclyl also includes a ring system wherein the heterocyclyl described above is fused with one or more cycloalkyl groups, wherein the point of attachment is on the heterocyclyl ring, or the heterocyclyl described above is fused with one or more aryl or heteroaryl groups, wherein the point of attachment is on the heterocyclyl ring; and in such cases, the number of ring members continues to represent the number of ring members in the heterocyclyl ring system. The heterocyclyl further comprises the heterocyclyl described above, in which the substituents on any non-adjacent carbon or nitrogen atoms are connected to form a bridge ring, together forming a polycyclic heteroalkane sharing two or more carbon or nitrogen atoms. The heterocyclyl further comprises the heterocyclyl described above, in which the substituents on the same carbon atom are connected to form a ring, together forming a polycyclic heteroalkane sharing one carbon atom. Exemplary 3-membered heterocyclyl groups containing one heteroatom include, but are not limited to, aziridinyl, oxiranyl and thiorenyl. Exemplary 4-membered heterocyclyl groups containing one heteroatom include, but are not limited to, azetidinyl, oxetanyl and thietanyl. Exemplary 5-membered heterocyclyl groups containing one heteroatom include, but are not limited to, tetrahydrofuranyl, dihydrofuranyl, tetrahydrothiophenyl, dihydrothienyl, pyrrolidinyl, dihydropyrrolyl and pyrrolyl-2,5-dione. Exemplary 5-membered heterocyclyl groups containing two heteroatoms include, but are not limited to, pyrazolidyl, dioxolanyl, oxasulfuranyl, disulfuranyl, and oxazolidin-2-one. Exemplary 5-membered heterocyclyl groups containing three heteroatoms include, but are not limited to, triazolinyl, oxadiazolinyl, and thiadiazolinyl. Exemplary 6-membered heterocyclyl groups containing one heteroatom include, but are not limited to, piperidyl, tetrahydropyranyl, dihydropyridyl and thianyl. Exemplary 6-membered heterocyclyl groups containing two heteroatoms include, but are not limited to, piperazinyl, morpholinyl, dithianyl and dioxanyl. Exemplary 6-membered heterocyclyl groups containing three heteroatoms include, but are not limited to, triazinanyl. Exemplary 7-membered heterocyclyl groups containing one heteroatom include, but are not limited to, azepanyl, oxepanyl and thiepanyl. Exemplary 5-membered heterocyclyl groups fused with a C₆ aryl (also referred to as 5,6-bicyclic heterocyclyl herein) include, but are not limited to, indolinyl, isoindolinyl, dihydrobenzofuranyl, dihydrobenzothiophenyl, benzoxazolinonyl, etc. Exemplary 6-membered heterocyclyl groups fused with a C₆ aryl (also referred to as 6,6-bicyclic heterocyclyl herein) include, but are not limited to, tetrahydroquinolinyl, tetrahydroisoquinolinyl, etc. The heterocyclyl further includes the heterocyclyl described above sharing one or two atoms with a cycloalkyl, heterocyclyl, aryl or heteroaryl to form a bridged or spiro ring, as long as the valence permits, where the shared atom may be carbon or nitrogen atoms. The heterocyclyl further includes the heterocyclyl described above, which optionally can be substituted with one or more substituents, e.g., with 1 to 5 substituents, 1 to 3 substituents or 1 substituent.

“C₆₋₁₀ aryl” refers to a radical of monocyclic or polycyclic (e.g., bicyclic) 4n+2 aromatic ring system (e.g., having 6 or 10 shared π electrons in a cyclic array) having 6-10 ring carbon atoms and zero heteroatoms. In some embodiments, the aryl group has six ring carbon atoms (“C₆ aryl”; for example, phenyl). In some embodiments, the aryl group has ten ring carbon atoms (“C₁₀ aryl”; for example, naphthyl, e.g., 1-naphthyl and 2-naphthyl). The aryl group also includes a ring system in which the aryl ring described above is fused with one or more cycloalkyl or heterocyclyl groups, and the point of attachment is on the aryl ring, in which case the number of carbon atoms continues to represent the number of carbon atoms in the aryl ring system. The aryl can be substituted with one or more substituents, for example, with 1 to 5 substituents, 1 to 3 substituents or 1 substituent.

“5-10 membered heteroaryl” refers to a radical of 5-10 membered monocyclic or bicyclic 4n+2 aromatic ring system (e.g., having 6, 10 or 14 shared π electrons in a cyclic array) having ring carbon atoms and 1-4 ring heteroatoms, wherein each heteroatom is independently selected from at least one of nitrogen, oxygen and sulfur. In the heteroaryl group containing one or more nitrogen atoms, the point of attachment can be a carbon or nitrogen atom as long as the valence permits. Heteroaryl bicyclic systems may include one or more heteroatoms in one or two rings. Heteroaryl also includes ring systems wherein the heteroaryl ring described above is fused with one or more cycloalkyl or heterocyclyl groups, and the point of attachment is on the heteroaryl ring. In such case, the number of the carbon atoms continues to represent the number of carbon atoms in the heteroaryl ring system. In other some embodiments, 5-6 membered heteroaryl groups are especially preferred, which are radicals of 5-6 membered monocyclic or bicyclic 4n+2 aromatic ring systems having ring carbon atoms and 1-4 ring heteroatoms. Exemplary 5-membered heteroaryl groups containing one heteroatom include, but are not limited to, pyrrolyl, furyl and thienyl. Exemplary 5-membered heteroaryl groups containing two heteroatoms include, but are not limited to, imidazolyl, pyrazolyl, oxazolyl, isoxazolyl, thiazolyl, and isothiazolyl. Exemplary 5-membered heteroaryl groups containing three heteroatoms include, but are not limited to, triazolyl, oxadiazolyl (such as, 1,2,4-oxadiazolyl), and thiadiazolyl. Exemplary 5-membered heteroaryl groups containing four heteroatoms include, but are not limited to, tetrazolyl. Exemplary 6-membered heteroaryl groups containing one heteroatom include, but are not limited to, pyridyl or pyridonyl. Exemplary 6-membered heteroaryl groups containing two heteroatoms include, but are not limited to, pyridazinyl, pyrimidinyl, and pyrazinyl. Exemplary 6-membered heteroaryl groups containing three or four heteroatoms include, but are not limited to, triazinyl and tetrazinyl. Exemplary 7-membered heteroaryl groups containing one heteroatom include, but are not limited to, azacycloheptatrienyl, oxepinyl, and thiepinyl. Exemplary 5,6-bicyclic heteroaryl groups include, but are not limited to, indolyl, isoindolyl, indazolyl, benzotriazolyl, benzothiophenyl, isobenzothiophenyl, benzofuranyl, benzoisofuranyl, benzimidazolyl, benzoxazolyl, benzoisoxazolyl, benzoxadiazolyl, benzothiazolyl, benzoisothiazolyl, benzothiadiazolyl, indolizinyl and purinyl. Exemplary 6,6-bicyclic heteroaryl groups include, but are not limited to, naphthyridinyl, pteridinyl, quinolyl, isoquinolyl, cinnolinyl, quinoxalinyl, phthalazinyl and quinazolinyl. The heteroaryl can be substituted with one or more substituents, for example, with 1 to 5 substituents, 1 to 3 substituents or 1 substituent.

“C₆₋₁₀ arylene” refers to a divalent group formed by removing another hydrogen of the C₆₋₁₀ aryl, and can be a substituted or unsubstituted arylene. In some embodiments, phenylene is preferred, such as

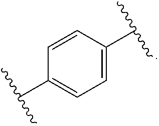 [SMILES: *c1ccc(*)cc1]

“C₅₋₁₀ heteroarylene” refers to a divalent group formed by removing another hydrogen of the C₅₋₁₀ heteroaryl, and can be a substituted or unsubstituted haloarylene. In some embodiment, C₅₋₉ heteroarylene, C₅₋₆ heteroarylene, C₆ heteroarylene and C₅ heteroarylene are especially preferred, such as

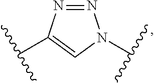 [SMILES: *c1cn(*)nn1]

such as

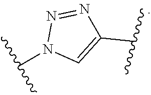 [SMILES: *c1cn(*)nn1]

The divalent groups formed by removing another hydrogen from the groups defined above such as alkyl, alkenyl, alkynyl, cycloalkyl, heterocyclyl, aryl and heteroaryl are collectively referred to as “ylene”. Ring-forming groups such as cycloalkyl, heterocyclyl, aryl and heteroaryl are collectively referred to as “cyclic groups”.

Alkyl, alkenyl, alkynyl, carbocyclyl, heterocyclyl, aryl and heteroaryl as defined herein are optionally substituted groups.

Exemplary substituents on carbon atoms include, but are not limited to, halogen, —C≡N, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂—, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), alkyl, haloalkyl, alkenyl, alkynyl, carbocyclyl, heterocyclyl, aryl and heteroaryl, wherein each of the alkyl, alkenyl, alkynyl, carbocyclyl, heterocyclyl, aryl and heteroaryl is independently substituted with 0, 1, 2, 3, 4 or 5 R^{dd} groups.

Or two geminal hydrogens on a carbon atom are replaced with ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb} or ═NOR^{cc} groups.

Each of the R^{aa} is independently selected from at least one of alkyl, haloalkyl, alkenyl, alkynyl, carbocyclyl, heterocyclyl, aryl and heteroaryl, or two of the R^{aa} groups are combined to form a heterocyclyl or heteroaryl ring, wherein each of the alkyl, alkenyl, alkynyl, carbocyclyl, heterocyclyl, aryl and heteroaryl is independently substituted with 0, 1, 2, 3, 4 or 5 R^{dd} groups.

Each of the R^{bb} is independently selected from at least one of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SO₂R^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, carbocyclyl, heterocyclyl, aryl and heteroaryl, or two R^{bb} groups are combined to form a heterocyclyl or a heteroaryl ring, wherein each of the alkyl, alkenyl, alkynyl, carbocyclyl, heterocyclyl, aryl and heteroaryl is independently substituted with 0, 1, 2, 3, 4 or 5 R^{dd} groups.

Each of the R^{cc} is independently selected from at least one of hydrogen, alkyl, haloalkyl, alkenyl, alkynyl, carbocyclyl, heterocyclyl, aryl and heteroaryl, or two R^{cc} groups are combined to form a heterocyclyl or a heteroaryl ring, wherein each of the alkyl, alkenyl, alkynyl, carbocyclyl, heterocyclyl, aryl and heteroaryl is independently substituted with 0, 1, 2, 3, 4 or 5 R^{dd} groups.

Each of the R^{dd} is independently selected from at least one of halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{cc}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, alkyl, haloalkyl, alkenyl, alkynyl, carbocyclyl, heterocyclyl, aryl, and heteroaryl, wherein each of the alkyl, alkenyl, alkynyl, carbocyclyl, heterocyclyl, aryl and heteroaryl is independently substituted with 0, 1, 2, 3, 4 or 5 R^{gg} groups, or two geminal R^{dd} substituents can be combined to form ═O or ═S.

Each of the R^{ee} is independently selected from at least one of alkyl, haloalkyl, alkenyl, alkynyl, carbocyclyl, aryl, heterocyclyl, and heteroaryl, wherein each of the alkyl, alkenyl, alkynyl, carbocyclyl, heterocyclyl, aryl and heteroaryl is independently substituted with 0, 1, 2, 3, 4 or 5 R^{gg} groups.

Each of the R^{ff} is independently selected from at least one of hydrogen, alkyl, haloalkyl, alkenyl, alkynyl, carbocyclyl, heterocyclyl, aryl and heteroaryl, or two R^{ff} groups are combined to form a heterocyclyl or a heteroaryl ring, wherein each of the alkyl, alkenyl, alkynyl, carbocyclyl, heterocyclyl, aryl and heteroaryl is independently substituted with 0, 1, 2, 3, 4 or 5 R^{gg} groups.

Each of the R^{gg} is independently selected from at least one of halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alky)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂C₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂-C₆ alkenyl, C₂-C₆ alkynyl, C₃-C₇ carbocyclyl, C₆-C₁₀ aryl, C₃-C₇ heterocyclyl, and C₅-C₁₀ heteroaryl; or two geminal R^{gg} substituents may combine to form ═O or ═S; wherein X⁻ is a counter-ion.

Exemplary substituents on nitrogen atoms include, but are not limited to at least one of hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, alkyl, haloalkyl, alkenyl, alkynyl, carbocyclyl, heterocyclyl, aryl and heteroaryl, or two R^{cc} groups attached to a nitrogen atom combine to form a heterocyclyl or a heteroaryl ring, wherein each of the alkyl, alkenyl, alkynyl, carbocyclyl, heterocyclyl, aryl and heteroaryl is independently substituted with 0, 1, 2, 3, 4 or 5 R^{dd} groups, and wherein R^{aa}, R^{bb}, R^{cc} and R^{dd} are as described herein.

### Other Definition

The term “oligonucleotide strand” refers to an oligomeric compounds with several or all chemical modified or without modified nucleotides, in which comprising a length of less than about 100 nucleotides (such as 1-20 nucleotides or 1-50 nucleotides). In some embodiments, oligonucleotide strand can include non-nucleic acid conjugated group. In some embodiments, the oligonucleotides comprise ribonucleic acid (RNA), deoxynucleic acid (DNA) or peptide nucleic acid (PNA). In some embodiments, the oligonucleotide strand is double strand or single strand. In some embodiments, the oligonucleic strand is siRNA, nucleic acid aptamer, antisense nucleic acid, sgRNA, tractRNA or crRNA.

The term “conjugate group” refers to an atom or atom group binding to oligonucleotide strand. In some situation, the conjugate group changes one or several properties of oligonucleotide that they bound to, including, but not limited to, pharmacodynamics, pharmacokinetics, binding, absorption, cellular distribution, cellular uptake, charge and/or scavenging properties.

The term “conjugate” refers to a coupling molecule of the compound described herein binding to oligonucleotide strand.

The term “receptor” refers to a biomacromolecule formed by glycoprotein or lipoprotein, which exists in cell membrane, cytoplasm or nucleus, different receptors have specific structure and configuration.

The term “ligand” refers to a substance or a compound having identification ability to receptor and is able to bind to it. In some embodiments, the said ligand is the ligand which binds to asialoglycoprotein receptor (ASGPR). In some embodiments, the said ligand is carbohydrate, such as monosaccharides and/or polysaccharides, including, but not limited to: galactose, N-acetylgalactosamine, mannose, glucose, glucosamine and fucose, including, but not limited to: D-mannopyranose, L-mannopyranose, D-arabinose, D-xylofuranose, L-xylofuranose, D-glucose, L-glucose, D-galactose, L-galactose, α-D-mannofuranose, β-D-mannofuranose, β-D-mannopyranose, α-D-glucopyranose, β-D-glucopyranose, α-D-glucofuranose, β-D-glucofuranose, α-D-fructofuranose, α-D-fructopyranose, α-D-galactopyranose, β-D-galactopyranose, α-D-galactofuranose, β-D-galactofuranose, glucosamine, sialic acid, galactosamine, N-acetylgalactosamine, N-trifluoroacetylgalactosamine, N-propionylgalactosamine, N-n-butyrylgalactosamine, N-isobutyrylgalactosamine, 2-amino-3-O—[(R)-1-carboxyethyl]-2-deoxy-β-D-glucopyranose, 2-deoxy-2-methylamino-L-glucopyranose, 4,6-dideoxy-4-carboxamido-2,3-di-O-methyl-D-mannopyranose, 2-deoxy-2-sulfoamino-D-glucopyranose, N-glycolyl-α-neuraminic acid, 5-thio-β-D-glucopyranose, 2,3,4-tri-O-acetyl-1-thio-6-O-tribenzyl-α-D-glucopyranosyl methyl ester, 4-thio-β-D-galactopyranose, 3,4,6,7-tetra-O-acetyl-2-deoxy-1,5-dithio-α-D-glucoheptoside ethyl ester, 2,5-anhydro-D-allosenitrile, ribose, D-ribose, D-4-thioribose, L-ribose and L-4-thioribose.

As used herein, the term “polysaccharide” refers to a polymer formed by several monosaccharides binding together by glycosidic bond. In this disclosure, the polysaccharide comprises oligosaccharide and/or oligomeric saccharide. Usually, “oligosaccharide” refers to a polymer formed by 2-10 monosaccharides binding together by glycosidic bond, “oligomeric saccharide” refers to a polymer formed by less than 20 monosaccharides binding together by glycosidic bond.

The terms “protecting group” and “protecting groups” refer to any atom or group of atoms that is added to a molecule in order to prevent existing groups in the molecule from undergoing unwanted chemical reactions. A “protecting group” may be a labile chemical moiety that is known in the art to protect reactive groups, such as hydroxyl, amino or thiol groups, against undesired or untimely reactions during chemical synthesis. Protecting groups are typically used selectively and/or orthogonally to protect sites during reactions at other reactive sites and can then be removed to leave the unprotected group as such or available for further reactions.

A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; a substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl)ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein). Preferred protecting groups are selected from a group comprising acetyl (Ac), benzoyl (Bzl), benzyl (Bn), isobutyryl (iBu), phenylacetyl, benzyloxymethyl acetal (BOM), beta-methoxyethoxymethyl ether (MEM), methoxymethylether (MOM), p-methoxybenzyl ether (PMB), methylthiomethyl ether, pivaloyl (Piv), tetrahydropyranyl (THP), triphenylmethyl (Trt), methoxytrityl [(4-methoxyphenyl)diphenylmethyl] (MMT), dimethoxytrityl, [bis-(4-methoxyphenyl)phenylmethyl (DMT), trimethylsilyl ether (TMS), tert-butyldimethylsilyl ether (TBDMS), tri-iso-propylsilyloxymethyl ether (TOM), tri-isopropylsilyl ether (TIPS), methyl ethers, ethoxyethyl ethers (EE), N,N-dimethylformamidine and 2-cyanoethyl (CE).

The term “hydroxyl protecting group” refers to groups in preventing hydroxyl from chemical reactions and deprotecting in specific condition to restore hydroxyl, which mainly comprises silane protecting groups, acyl protecting groups or ether protecting groups, preferred selected from at least one of:
trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethyloxycarbonyl (Fmoc), allyloxycarbonyl (Alloc), 2,2,2-trichloroethoxycarbonyl (Troc), carbobenzyloxy (Cbz), tert-butyloxycarbonyl (Boc), benzyl (Bn), p-methoxybenzyl (PMB), allyl, dimethoxytrityl (DMT), monomethoxytrityl (MMT), 9-phenyloxaanthracen-9-yl (Pixyl), 9-(p-methoxyphenyl) oxaanthracen-9-yl (Mox), trityl (Tr), 4-methoxytriphenylmethyl (MMTr), 4,4′-dimethoxytrityl (DMTr), 4,4′,4″-trimethoxytriphenylmethyl (TMTr), methoxymethyl (MOM), phenoxymethyl (BOM), 2,2,2-trichloroethoxymethyl, 2-methoxyethoxymethyl (MEM), methylthiomethyl (MTM), and p-methoxybenzyloxymethyl (PMBM).

In some embodiments, the hydroxyl protecting group is preferably selected from at least one of:
dimethoxytrityl (DMT), monomethoxytrityl (MMT), 9-phenyloxaanthracen-9-yl (Pixyl), 9-(p-methoxyphenyl) oxaanthracen-9-yl (Mox), trityl (Tr), 4-methoxytriphenylmethyl (MMTr), 4,4′-dimethoxytrityl (DMTr), 4,4′,4″-trimethoxytriphenylmethyl (TMTr), tert-butyldimethylsilyl (TBDMS), tert-butyldiphenylsilyl (TBDPS), triisopropylsilyl (TIPS), preferably selected from dimethoxytrityl (DMT), monomethoxytrityl (MMT), trityl (Tr), 4-methoxytriphenylmethyl (MMTr), 4,4′-dimethoxytrityl (DMTr), 4,4′,4″-trimethoxytriphenylmethyl (TMTr), preferably is 4,4′-dimethoxytrityl (DMTr).

In some embodiments, the hydroxyl protecting group is preferably selected from at least one of:
trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS) and triisopropylsilyl (TIPS), preferably tert-butyldimethylsilyl (TBDMS).

The term “carboxyl protecting group” refers to groups in preventing carboxyl from chemical reactions and deprotecting in specific condition to restore carboxyl, which mainly comprises protecting groups form with carboxyl to be esters. The said carboxyl protecting group is preferably selected from at least one of:

C₁₋₆ alkyl, benzyl, allyl,

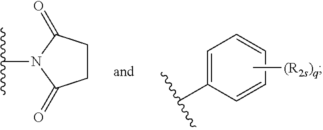

preferably, the said C₁₋₆ alkyl can be unsubstituted, or can be substituted, including, but not limited to: methyl, substituted methyl, β-substituted ethyl, and tert-butyl and so on.

The term “amino protecting group” refers to groups in preventing amino from chemical reactions and deprotecting in specific condition to restore amino, including, but not limited to: benzyloxycarbonyl (Cbz), tert-butoxycarbonyl (Boc), 9-fluorenylmethyloxycarbonyl (Fmoc), allyloxycarbonyl (Alloc), 2-(trimethylsilyl)ethoxycarbonyl (Teoc), methoxycarbonyl and ethoxycarbonyl and so on. In some embodiments, tert-butoxycarbonyl (Boc) is preferred.

The term “condensation activator” refers to materials could accelerate reaction rate of condensation reaction.

As used herein, the term “about” should be understood by a person skilled in the art, and varies to a certain extent depending on the context. If the use of the term is not clear for a person skilled in the art based the context, the term “about” means the deviation is no more than ±10% of the specific numerical value or range.

The term “treating” as used herein relates to reversing, alleviating or inhibiting the progression or prevention of the disorders or conditions to which the term applies, or of one or more symptoms of such disorders or conditions. The noun “treatment” as used herein relates to the action of treating, which is a verb, and the latter is as just defined.

The term “pharmaceutically acceptable salt” as used herein refers to those carboxylate and amino acid addition salts of the compounds of the present disclosure, which are suitable for the contact with patients' tissues within a reliable medical judgment, and do not produce inappropriate toxicity, irritation, allergy, etc. They are commensurate with a reasonable benefit/risk ratio, and are effective for their intended use. The term includes, if possible, the zwitterionic form of the compounds of the disclosure.

The pharmaceutically acceptable base addition salts are formed with metals or amines, such as alkali metal and alkaline earth metal hydroxides or organic amines. Examples of the metals used as cations include sodium, potassium, magnesium, calcium, etc. Examples of suitable amines are N,N′-dibenzylethylenediamine, chloroprocaine, choline, diethanolamine, ethylenediamine, N-methylglucamine and procaine.

The base addition salt of the acidic compound can be prepared by contacting the free acid form with a sufficient amount of the required base to form a salt in a conventional manner. The free acid can be regenerated by contacting the salt form with an acid in a conventional manner and then isolating the free acid. The free acid forms are somewhat different from their respective salt forms in their physical properties, such as solubility in polar solvents. But for the purposes of the present disclosure, the salts are still equivalent to their respective free acids.

The salts can be prepared from the inorganic acids, which include sulfates, pyrosulfates, bisulfates, sulfites, bisulfites, nitrates, phosphates, monohydrogen phosphates, dihydrogen phosphates, metaphosphates, pyrophosphates, chlorides, bromides and iodides. Examples of the acids include hydrochloric acid, nitric acid, sulfuric acid, hydrobromic acid, hydroiodic acid, phosphoric acid, etc. The representative salts include hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthoate, methanesulfonate, glucoheptanate, lactobionate, lauryl sulfonate, isethionate, etc. The salts can also be prepared from the organic acids, which include aliphatic monocarboxylic and dicarboxylic acids, phenyl-substituted alkanoic acids, hydroxyalkanoic acids, alkanedioic acid, aromatic acids, aliphatic and aromatic sulfonic acids, etc. The representative salts include acetate, propionate, octanoate, isobutyrate, oxalate, malonate, succinate, suberate, sebacate, fumarate, maleate, mandelate, benzoate, chlorobenzoate, methyl benzoate, dinitrobenzoate, naphthoate, besylate, tosylate, phenylacetate, citrate, lactate, maleate, tartrate, methanesulfonate, etc. The pharmaceutically acceptable salts can include cations based on alkali metals and alkaline earth metals, such as sodium, lithium, potassium, calcium, magnesium, etc., as well as non-toxic ammonium, quaternary ammonium, and amine cations including, but not limited to, ammonium, tetramethylammonium, tetraethylammonium, methylamine, dimethylamine, trimethylamine, triethylamine, ethylamine, etc. Salts of amino acids are also included, such as arginine salts, gluconates, galacturonates, etc. (for example, see Berge S. M. et al., “Pharmaceutical Salts,” J. Pharm. Sci., 1977; 66: 1-19 for reference).

“Subjects” to which administration is contemplated include, but are not limited to, humans (e.g., males or females of any age group, e.g., pediatric subjects (e.g., infants, children, adolescents) or adult subjects (e.g., young adults, middle-aged adults or older adults) and/or non-human animals, such as mammals, e.g., primates (e.g., cynomolgus monkeys, rhesus monkeys), cattle, pigs, horses, sheep, goats, rodents, cats and/or dogs. In some embodiments, the subject is a human. In some embodiments, the subject is a non-human animal. The terms “human”, “patient” and “subject” can be used interchangeably herein.

“Disease”, “disorder”, and “condition” can be used interchangeably herein.

Unless otherwise indicated, the term “treatment” as used herein includes the effect on a subject who is suffering from a particular disease, disorder, or condition, which reduces the severity of the disease, disorder, or condition, or delays or slows the progression of the disease, disorder or condition (“therapeutic treatment”). The term also includes the effect that occurs before the subject begins to suffer from a specific disease, disorder or condition (“prophylactic treatment”).

Generally, the “effective amount” of a pharmaceutical composition refers to an amount sufficient to elicit a target biological response. As understood by those skilled in the art, the effective amount of the pharmaceutical composition of the disclosure can vary depending on the following factors, such as the desired biological objective, the pharmacokinetics of the compound, the diseases being treated, the mode of administration, and the age, health status and symptoms of the subjects. The effective amount includes therapeutically effective amount and prophylactically effective amount.

Unless otherwise indicated, the “therapeutically effective amount” of the pharmaceutical composition as used herein is an amount sufficient to provide therapeutic benefits in the course of treating a disease, disorder or condition, or to delay or minimize one or more symptoms associated with the disease, disorder or condition. The therapeutically effective amount of a compound refers to the amount of the therapeutic agent that, when used alone or in combination with other therapies, provides a therapeutic benefit in the treatment of a disease, disorder or condition. The term “therapeutically effective amount” can include an amount that improves the overall treatment, reduces or avoids the symptoms or causes of the disease or condition, or enhances the therapeutic effect of other therapeutic agents.

Unless otherwise indicated, the “prophylactically effective amount” of the compound as used herein is an amount sufficient to prevent a disease, disorder or condition, or an amount sufficient to prevent one or more symptoms associated with a disease, disorder or condition, or an amount sufficient to prevent the recurrence of a disease, disorder or condition. The prophylactically effective amount of a compound refers to the amount of a therapeutic agent that, when used alone or in combination with other agents, provides a prophylactic benefit in the prevention of a disease, disorder or condition. The term “prophylactically effective amount” can include an amount that improves the overall prevention, or an amount that enhances the prophylactic effect of other preventive agents.

“Combination” and related terms refer to the simultaneous or sequential administration of the pharmaceutical compositions of the present disclosure and other therapeutic agents. For example, the pharmaceutical compositions of the present disclosure can be administered simultaneously or sequentially in separate unit dosage with other therapeutic agents, or simultaneously in a single unit dosage with other therapeutic agents.

### DETAILED DESCRIPTION

The embodiments of the disclosure are described by combining the following examples. However, a person skilled in the art understands that the following examples are only intended to describe the disclosure, and should not be regarded as defining the scope of the disclosure. In the case where the concrete conditions are not indicated in the examples, the examples are carried out according to conventional conditions or the conditions recommended by the manufacturer. The reagents or apparatuses, the manufacturers of which are not indicated, are the conventional products that are commercially available.

As used herein, the term “compound disclosed herein” refers to the following compounds of formulae (I), formulae (II), or a pharmaceutically acceptable salt, isotopic variants, tautomers, stereoisomers, prodrugs, polymorphs, hydrates, or solvates thereof; “conjugate disclosed herein” refers to the following conjugates of formulae (III).

For compounds having an asymmetric center, it should be understood, unless otherwise stated, that all optical isomers and mixtures thereof are included. Furthermore, unless otherwise specified, all isomer compounds and carbon-carbon double bonds included in the present disclosure may occur in the form of Z and E. Compounds which exist in different tautomeric forms, one of which is not limited to any particular tautomer, but is intended to cover all tautomeric forms.

In one embodiment, this disclosure relates a compound of formula (I), or a pharmaceutical acceptable salt, tautomer or stereoisomer thereof:

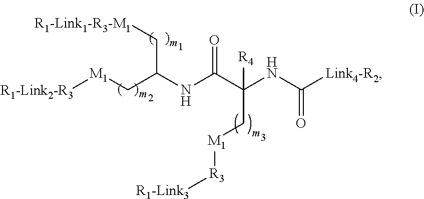

wherein,
M₁ is independently selected from at least one of —O—, —S—, —NR₆—, —CHR₇—, —C(O)—, —S(O)— and —S(O)₂—;
R₁ is residue of asialoglycoprotein receptor (ASGPR) ligand;
R₂ is selected from at least one of hydroxyl, carboxyl, —ORₚ, —OPG₂,

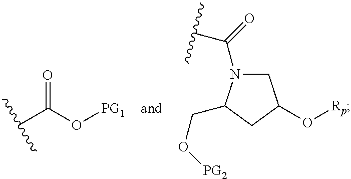

PG₁ is carboxyl protecting group;
PG₂ is hydroxyl protecting group;
Rₚ is

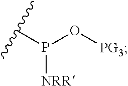

PG₃ is independently selected from phosphate protecting group;
R and R′ are respectively and independently selected from amino protecting group; or R, R′ and the N atom they linked optionally form together to be a 5-10 membered heterocyclic group;
R₃ is independently selected from

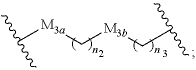

M₃ₐ is independently selected from at least one of chemical bond, —O—, —S—, —NR₆—, —CHR₇—, —C(O)—, —S(O)₁₋₂—, —NR₆C(O)—, —C(O)NR₆—, —NR₆S(O)₁₋₂—, —S(O)₁₋₂NR₆—, —OC(O)—, —C(O)O—, —OS(O)₁₋₂— and —S(O)₁₋₂O—;
M_{3b} is independently selected from at least one of —O—, —S—, —NR₆—, —CHR₇—, —CH═CH—, —C≡C—, —C(O)—, —S(O)₁₋₂—, —C(O)NR₆—, —NR₆C(O)—, —OC(O)—, —C(O)O—, —S(O)₁₋₂O—Y₁—, —OS(O)₁₋₂—Y₁—, —OS(O)₁₋₂O—, —S(O)₁₋₂NR₆—Y₁—, —NR₆S(O)₁₋₂—Y₁—, C₆₋₁₀ arylene, 5-10 membered heteroarylene,

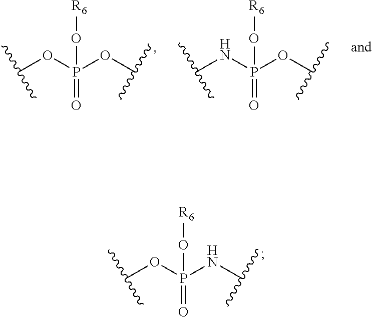

Y₁ is independently selected from C₆₋₁₀ arylene or 5-10 membered heteroarylene;
R₆ is respectively and independently selected from at least one of H, C₁₋₁₀ alkyl, C₁₋₁₀ haloalkyl, C₃₋₁₀ cycloalkyl, 3-10 membered heterocyclyl;
R₇ is respectively and independently selected from at least one of H, halogen, CN, NO₂, C₁₋₁₀ alkyl, C₁₋₁₀ haloalkyl, C₃₋₁₀ cycloalkyl, 3-10 membered heterocyclyl, C₆₋₁₀ arylene, 5-10 membered heteroaryl group, wherein, one or multiple methylene of the said alkyl or haloalkyl is optionally and independently substituted by the following group which is selected from at least one of —C(O)—, —C(O)NH—, —NHC(O)—, —OC(O)—, —O—, —C(O)O—, —S—, —S—(CH₂)₀₋₆—S—, —NR₆—, C₆₋₁₀ arylene, 5-10 membered heteroarylene, —CH═CH—, —C≡C— and —O—(CH₂)₁₋₆—O—;
n₂ is independently selected from 0, 1, 2, 3, 4, 5, 6, 7, 8, 9 or 10;
n₃ is independently selected from 1, 2, 3, 4, 5 or 6;
R₄ is selected from at least one of H, C₁₋₆ alkyl, C₁₋₆ haloalkyl and C₁₋₆ alkoxy;
Link₁, Link₂, Link₅ and Link₄ are respectively and independently selected from C₁₋₂₅ linear chain alkylene, wherein, one or multiple methylene of the said linear chain alkylene is optionally and independently substituted by R*, R* is independently selected from at least one of —O—, —S—, —NR₆—, —CHR₇—, —C(O)—, —S(O)₁₋₂—, —NR₆C(O)—, —C(O)NR₆—, —NR₆S(O)₁₋₂—, —S(O)₁₋₂NR₆—, —OC(O)—, —C(O)O—, —OS(O)₁₋₂— and —S(O)₁₋₂O—;
m₁, m₂ and m₃ are respectively and independently selected from 1, 2, 3, 4 or 5;
with the proviso that the methylene connected to the carbonyl of left side in Link₄ is not substituted by R*.

M₁:

In one embodiment, M₁ is —O—; in other embodiment, M₁ is —S—; in other embodiment, M₁ is —NR₆—, such as —NH—; in other embodiment, M₁ is —CHR₇—, such as —CH₂—; in other embodiment, M₁ is —C(O)—; in other embodiment, M₁ is —S(O)—; in other embodiment, M₁ is —S(O)₂—.

In one specific embodiment, M₁ is independently selected from at least one of —O—, —S—, —NH—, —CH₂—, —C(O)—, —S(O)— and —S(O)₂—; in another more specific embodiment, M₁ is independently selected from at least one of —O—, —S— and —NH—; in another more specific embodiment, M₁ is —O—.

R₁:

In one embodiment, R₁ is residue of asialoglycoprotein receptor (ASGPR) ligand; in other embodiment, the said ASGPR ligand is D-mannopyranose; in other embodiment, the said ASGPR ligand is L-mannopyranose; in other embodiment, the said ASGPR ligand is L-arabinose; in other embodiment, the said ASGPR ligand is D-arabinose; in other embodiment, the said ASGPR ligand is D-xylofuranose; in other embodiment, the said ASGPR ligand is L-xylofuranose; in other embodiment, the said ASGPR ligand is D-glucose; in other embodiment, the said ASGPR ligand is L-glucose; in other embodiment, the said ASGPR ligand is D-galactose; in other embodiment, the said ASGPR ligand is L-galactose; in other embodiment, the said ASGPR ligand is α-D-mannofuranose; in other embodiment, the said ASGPR ligand is β-D-mannofuranose; in other embodiment, the said ASGPR ligand is α-D-mannopyranose; in other embodiment, the said ASGPR ligand is β-D-mannopyranose; in other embodiment, the said ASGPR ligand is α-D-glucopyranose; in other embodiment, the said ASGPR ligand is β-D-glucopyranose; in other embodiment, the said ASGPR ligand is α-D-glucofuranose; in other embodiment, the said ASGPR ligand is β-D-glucofuranose; in other embodiment, the said ASGPR ligand is α-D-fructofuranose; in other embodiment, the said ASGPR ligand is β-D-fructofuranose; in other embodiment, the said ASGPR ligand is α-D-fructopyranose; in other embodiment, the said ASGPR ligand is β-D-fructopyranose; in other embodiment, the said ASGPR ligand is α-D-galactopyranose; in other embodiment, the said ASGPR ligand is β-D-galactopyranose; in other embodiment, the said ASGPR ligand is α-D-galactofuranose; in other embodiment, the said ASGPR ligand is β-D-galactofuranose; in other embodiment, the said ASGPR ligand is glucosamine; in other embodiment, the said ASGPR ligand is sialic acid; in other embodiment, the said ASGPR ligand is galactosamine; in other embodiment, the said ASGPR ligand is N-acetylgalactosamine; in other embodiment, the said ASGPR ligand is N-trifluoroacetylgalactosamine; in other embodiment, the said ASGPR ligand is N-propionylgalactosamine; in other embodiment, the said ASGPR ligand is N-n-butyrylgalactosamine; in other embodiment, the said ASGPR ligand is N-isobutyrylgalactosamine; in other embodiment, the said ASGPR ligand is 2-amino-3-O—[(R)-1-carboxyethyl]-2-deoxy-β-D-glucopyranose; in other embodiment, the said ASGPR ligand is 2-deoxy-2-methylamino-L-glucopyranose; in other embodiment, the said ASGPR ligand is 4,6-dideoxy-4-carboxamido-2,3-di-O-methyl-D-mannopyranose; in other embodiment, the said ASGPR ligand is 2-deoxy-2-sulfoamino-D-glucopyranose; in other embodiment, the said ASGPR ligand is N-glycolyl-α-neuraminic acid; in other embodiment, the said ASGPR ligand is 5-thio-β-D-glucopyranose; in other embodiment, the said ASGPR ligand is 2,3,4-tri-O-acetyl-1-thio-6-O-tribenzyl-α-D-glucopyranosyl methyl ester; in other embodiment, the said ASGPR ligand is 4-thio-3-D-galactopyranose; in other embodiment, the said ASGPR ligand is 3,4,6,7-tetra-O-acetyl-2-deoxy-1,5-dithio-α-D-glucoheptoside ethyl ester; in other embodiment, the said ASGPR ligand is 2,5-anhydro-D-allosenitrile; in other embodiment, the said ASGPR ligand is ribose; in other embodiment, the said ASGPR ligand is D-ribose; in other embodiment, the said ASGPR ligand is D-4-thioribose; in other embodiment, the said ASGPR ligand is L-ribose; in other embodiment, the said ASGPR ligand is L-4-thioribose.

In one specific embodiment, R₁ is independently selected from at least one of residue of the following structure: D-mannopyranose, L-mannopyranose, L-arabinose, D-arabinose, D-xylofuranose, L-xylofuranose, D-glucose, L-glucose, D-galactose, L-galactose, α-D-mannofuranose, β-D-mannofuranose, α-D-mannopyranose, β-D-mannopyranose, α-D-glucopyranose, β-D-glucopyranose, α-D-glucofuranose, β-D-glucofuranose, α-D-fructofuranose, β-D-fructofuranose, α-D-fructopyranose, β-D-fructopyranose, α-D-galactopyranose, β-D-galactopyranose, α-D-galactofuranose, β-D-galactofuranose, glucosamine, sialic acid, galactosamine, N-acetylgalactosamine, N-trifluoroacetylgalactosamine, N-propionylgalactosamine, N-n-butyrylgalactosamine, N-isobutyrylgalactosamine, 2-amino-3-O—[(R)-1-carboxyethyl]-2-deoxy-β-D-glucopyranose, 2-deoxy-2-methylamino-L-glucopyranose, 4,6-dideoxy-4-carboxamido-2,3-di-O-methyl-D-mannopyranose, 2-deoxy-2-sulfamino-D-glucopyranose, N-glycolyl-α-neuraminic acid, 5-thio-β-D-glucopyranose, 2,3,4-tri-O-acetyl-1-thio-6-O-tribenzyl-α-D-glucopyranosyl methyl ester, 4-thio-β-D-galactopyranose, 3,4,6,7-tetra-O-acetyl-2-deoxy-1,5-dithio-α-D-glucoheptoside ethyl ester, 2,5-anhydro-D-allosenitrile, ribose, D-ribose, D-4-thioribose, L-ribose and L-4-thioribose.

In other specific embodiment, R₁ is independently selected from at least one of residue of the following structure: D-galactose, L-galactose, α-D-galactopyranose, β-D-galactopyranose, α-D-galactofuranose, β-D-galactofuranose, galactosamine, N-acetylgalactosamine, N-trifluoroacetylgalactosamine, N-propionylgalactosamine, N-n-butyrylgalactosamine, N-isobutyrylgalactosamine and β-D-galactofuranose.

In other specific embodiment, R₁ is independently selected from at least one of residue of the following structure: galactosamine, N-acetylgalactosamine, N-trifluoroacetylgalactosamine, N-propionylgalactosamine, N-n-butyrylgalactosamine, N-isobutyrylgalactosamine.

In other specific embodiment, R₁ is residue of N-acetylgalactosamine.

In other specific embodiment, R₁ is

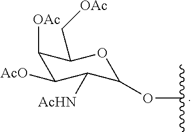 [SMILES: *OC1OC(COC(C)=O)C(OC(C)=O)C(OC(C)=O)C1NC(C)=O]

R₂:

In one embodiment, R₂ is hydroxyl; in other embodiment, R₂ is carboxyl; in other embodiment, R₂ is —ORₚ, such as

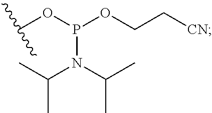 [SMILES: *OP(OCCC#N)N(C(C)C)C(C)C]

in other embodiment, R₂ is —OPG₂; in other embodiment, R₂ is

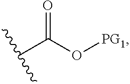

such as

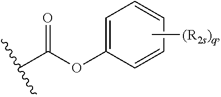

such as

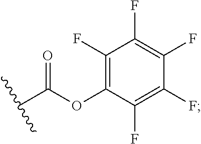 [SMILES: *C(=O)Oc1c(F)c(F)c(F)c(F)c1F]

in other embodiment, R₂ is

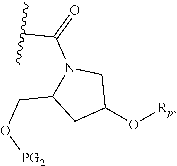

such as

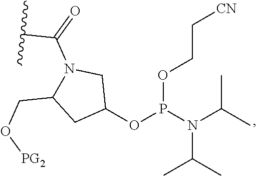

such as

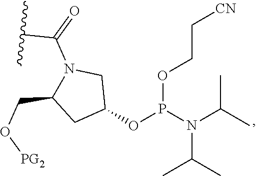

such as

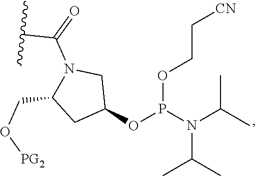

such as

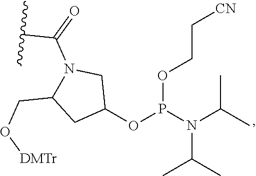

such as

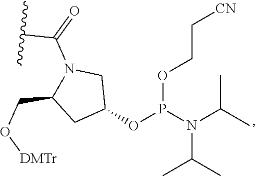

such as

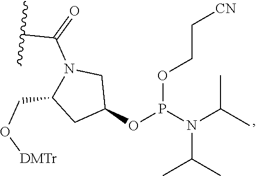

In one specific embodiment, R₂ is selected from at least one of hydroxyl, carboxyl, —ORₚ, —OPG₂,

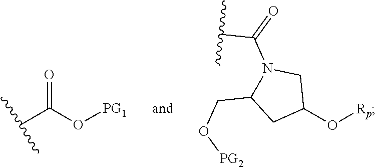

in other specific embodiment, R₂ is selected from at least one of —ORₚ,

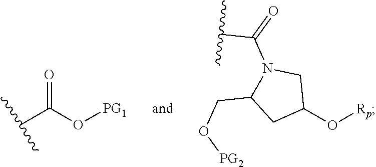

in other specific embodiment, R₂ is selected from at least one of

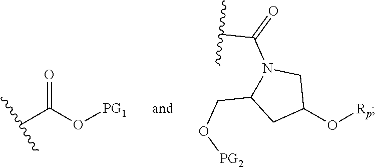

in other specific embodiment, R₂ is selected from at least one of

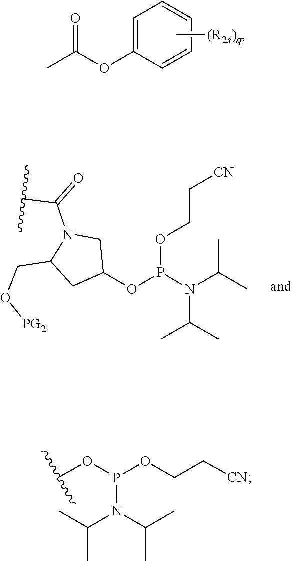

in other specific embodiment, R₂ is selected from

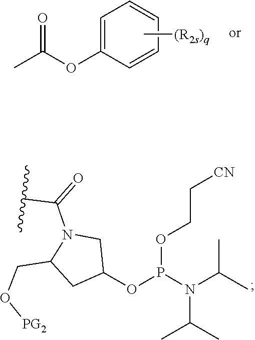

in other specific embodiment, R₂ is

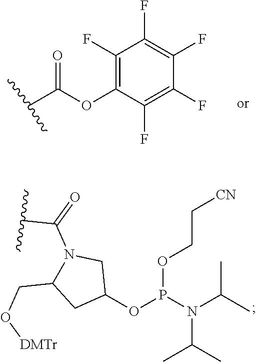

in other specific embodiment, R₂ is selected from at least one of

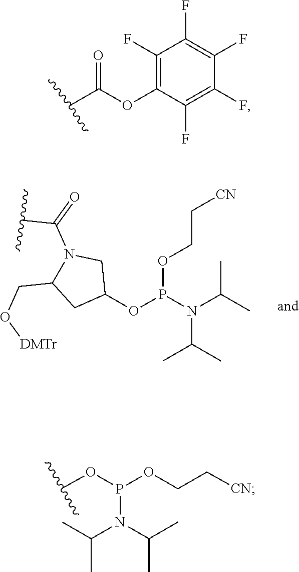

in other specific embodiment, R₂ is

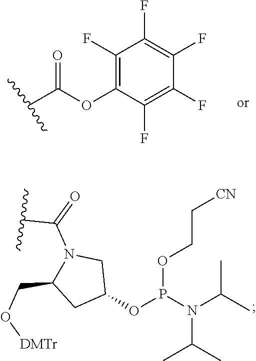

in other specific embodiment,

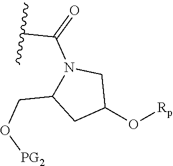

is

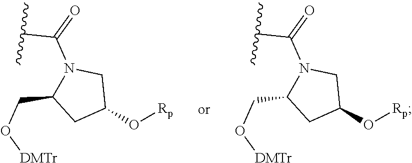

in other specific embodiment,

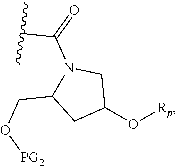

is

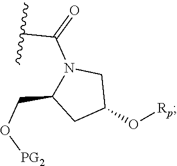

in other specific embodiment,

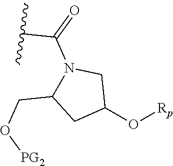

is

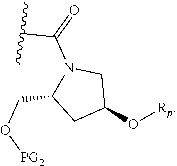

PG₁:

In one embodiment, PG₁ is carboxyl protecting group, such as C₁₋₆ alkyl, such as methyl, such as tert-butyl group, such as benzyl, such as allyl, such as

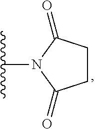 [SMILES: *N1C(=O)CCC1=O]

such as

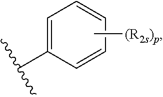

such as

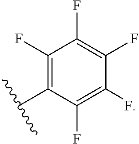 [SMILES: *c1c(F)c(F)c(F)c(F)c1F]

In one specific embodiment, PG₁ is selected from at least one of C₁₋₆ alkyl, benzyl, allyl,

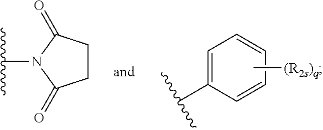

in other more specific embodiment, PG₁ is selected from at least one of methyl, tert-butyl group, benzyl, allyl,

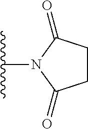 [SMILES: *N1C(=O)CCC1=O]

and

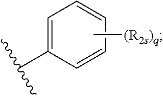

in other more specific embodiment, PG₁ is selected from at least one of

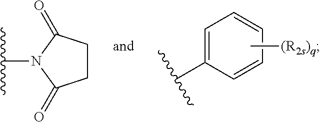

in other more specific embodiment, PG₁ is

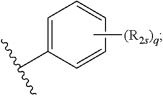

in other more specific embodiment, PG₁ is

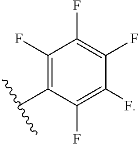 [SMILES: *c1c(F)c(F)c(F)c(F)c1F]

In one embodiment, R₂ₛ is H; in other embodiment, R₂ₛ is halogen, such as F; in other embodiment, R₂ₛ is C₁₋₁₀ alkyl, such as C₁₋₆ alkyl; in other embodiment, R₂ₛ is C₁₋₁₀ haloalkyl, such as C₁₋₆ haloalkyl.

In one specific embodiment, R₂ₛ is independently selected from at least one of H, halogen, C₁₋₁₀ alkyl and C₁₋₁₀ haloalkyl; in other specific embodiment, R₂ₛ is independently selected from at least one of H, halogen, C₁₋₆ alkyl and C₁₋₆ haloalkyl; in other specific embodiment, R₂ₛ is H or halogen; in other specific embodiment, R₂ₛ is F.

In one embodiment, q is 0; in other embodiment, q is 1; in other embodiment, q is 2; in other embodiment, q is 3; in other embodiment, q is 4; in other embodiment, q is 5.

PG₂:

In one embodiment, PG₂ is hydroxyl protecting group, such as trimethylsilyl (TMS), such as triethylsilyl (TES), such as dimethylisopropylsilyl (DMIPS), such as diethylisopropylsilyl (DEIPS), such as tert-butyldimethylsilyl (TBDMS), such as tert-butyldiphenylsilyl (TBDPS), such as triisopropylsilyl (TIPS), such as acetyl (Ac), such as chloroacetyl, such as dichloroacetyl, such as trichloroacetyl, such as trifluoroacetyl (TFA), such as benzoyl, such as p-methoxybenzoyl, such as 9-fluorenylmethyloxycarbonyl (Fmoc), such as allyloxycarbonyl (Alloc), such as 2,2,2-trichloroethoxycarbonyl (Troc), such as carbobenzyloxy (Cbz), such as tert-butyloxycarbonyl (Boc), such as benzyl (Bn), such as p-methoxybenzyl (PMB), such as allyl, such as dimethoxytrityl (DMT), such as monomethoxytrityl (MMT), such as 9-phenyl oxaanthracen-9-yl (Pixyl), such as 9-(p-methoxyphenyl) oxaanthracen-9-yl (Mox), such as trityl (Tr), such as 4-methoxytriphenylmethyl (MMTr), such as 4,4′-dimethoxytrityl (DMTr), such as 4,4′,4″-trimethoxytriphenylmethyl (TMTr), such as methoxymethyl (MOM), such as phenoxymethyl (BOM), such as 2,2,2-trichloroethoxymethyl, such as 2-methoxyethoxymethyl (MEM), such as methylthiomethyl (MTM), and p-methoxybenzyloxymethyl (PMBM).

In one specific embodiment, PG₂ is selected from at least one of trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethyloxycarbonyl (Fmoc), allyloxycarbonyl (Alloc), 2,2,2-trichloroethoxycarbonyl (Troc), carbobenzyloxy (Cbz), tert-butyloxycarbonyl (Boc), benzyl (Bn), p-methoxybenzyl (PMB), allyl, dimethoxytrityl (DMT), monomethoxytrityl (MMT), 9-phenyloxaanthracen-9-yl (Pixyl), 9-(p-methoxyphenyl) oxaanthracen-9-yl (Mox), trityl (Tr), 4-methoxytriphenylmethyl (MMTr), 4,4′-dimethoxytrityl (DMTr), 4,4′,4″-trimethoxytriphenylmethyl (TMTr), methoxymethyl (MOM), phenoxymethyl (BOM), 2,2,2-trichloroethoxymethyl, 2-methoxyethoxymethyl (MEM), methylthiomethyl (MTM), and p-methoxybenzyloxymethyl (PMBM);
In other specific embodiment, PG₂ is selected from at least one of: dimethoxytrityl (DMT), monomethoxytrityl (MMT), 9-phenyloxaanthracen-9-yl (Pixyl), 9-(p-methoxyphenyl) oxaanthracen-9-yl (Mox), trityl (Tr), 4-methoxytriphenylmethyl (MMTr), 4,4′-dimethoxytrityl (DMTr), 4,4′,4″-trimethoxytriphenylmethyl (TMTr), tert-butyldimethylsilyl (TBDMS), tert-butyldiphenylsilyl (TBDPS) and triisopropylsilyl (TIPS).

In other specific embodiment, PG₂ is selected from at least one of: dimethoxytrityl (DMT), monomethoxytrityl (MMT), trityl (Tr), 4-methoxytriphenylmethyl (MMTr), 4,4′-dimethoxytrityl (DMTr) and 4,4′,4″-trimethoxytriphenylmethyl (TMTr).

In other specific embodiment, PG₂ is 4,4′-dimethoxytrityl (DMTr).

Rₚ:

In one embodiment, Rₚ is

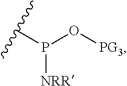

such as

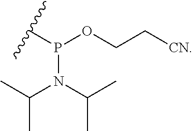 [SMILES: *P(OCCC#N)N(C(C)C)C(C)C]

PG₃:

In one embodiment, PG₃ is phosphate protecting group, such as 2-cyanoethyl, such as C₁₋₆ alkyl, such as methyl.

In one more specific embodiment, PG₃ is independently selected from at least one of 2-cyanoethyl and C₁₋₆ alkyl; in one more specific embodiment, PG₃ is independently selected from at least one of 2-cyanoethyl and methyl; in one more specific embodiment, PG₃ is 2-cyanoethyl.

R and R′:

In one embodiment, R and R′ are respectively and independently amino protecting group, such as C₁₋₆ alkyl, such as isopropyl, such as R, R′ and the N atom they linked optionally form together to be a 5-10 membered heterocyclic group.

R₃:

In one embodiment, R₃ is independently selected from

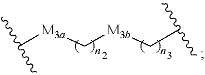

in other embodiment, R₃ is

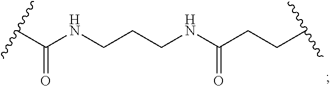 [SMILES: *CCC(=O)NCCCNC(*)=O]

in other embodiment, R₃ is

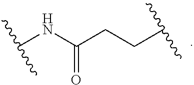 [SMILES: *CCC(=O)N*]

In one embodiment,

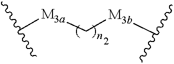

is A1; in other embodiment,

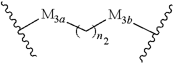

is A2; in other embodiment,

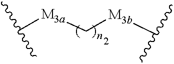

is A3; in other embodiment,

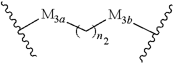

is A4; in other embodiment,

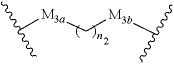

is A5; in other embodiment,

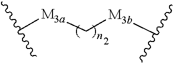

is A6; in other embodiment,

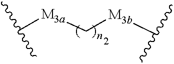

is A7; in other embodiment,

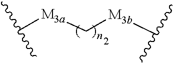

is A8; in other embodiment,

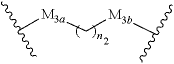

is A9; in other embodiment,

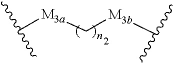

is A10; in other embodiment,

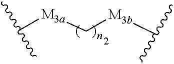

is A11; in other embodiment,

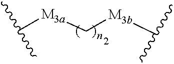

is A12; in other embodiment,

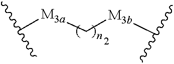

is A13; in other embodiment,

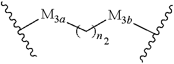

is A14; in other embodiment,

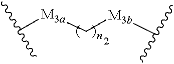

is A15; in other embodiment,

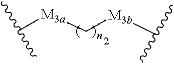

is A16; in other embodiment,

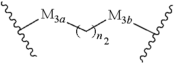

is A17; in other embodiment,

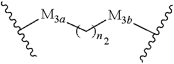

is A18; in other embodiment,

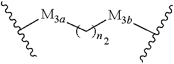

is A19; in other embodiment,

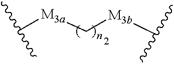

is A20; in other embodiment,

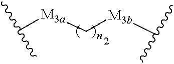

is A21; in other embodiment,

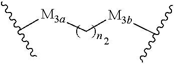

is A22; in other embodiment,

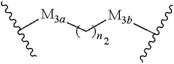

is A23; in other embodiment,

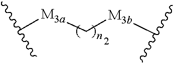

### is A24.

The structure of A1-A24 is shown as follows:

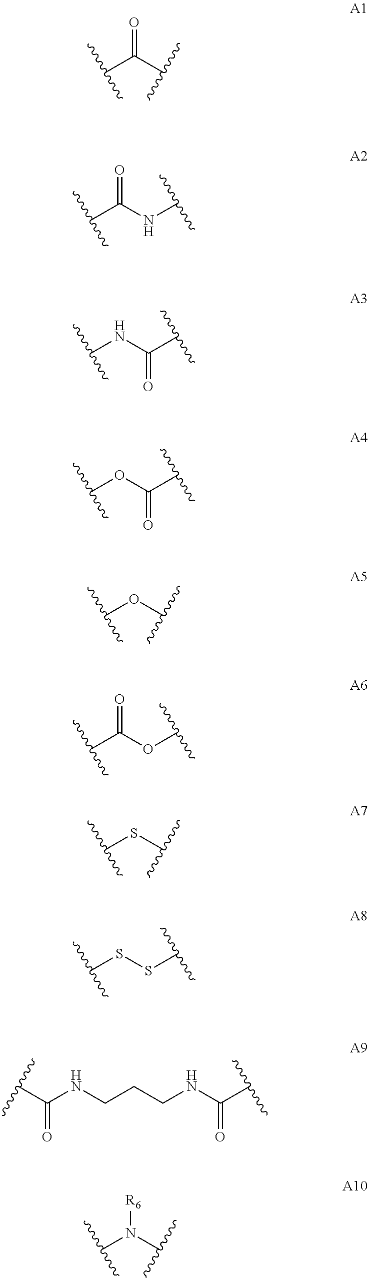

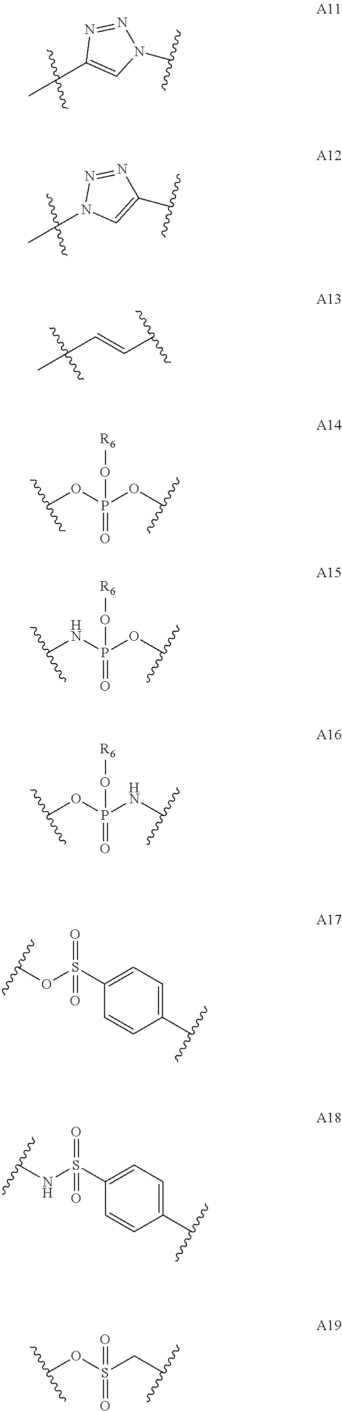

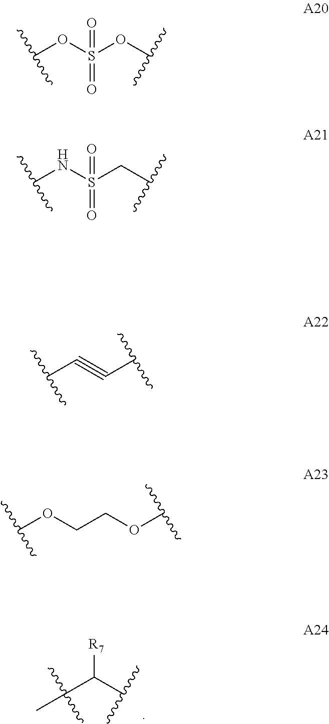

In one specific embodiment,

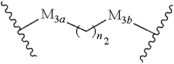

is independently selected from the structure of A1-A24; in other more specific embodiment,

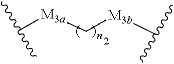

is independently selected from at least one of A2, A3, A4, A6 and A9; in other more specific embodiment,

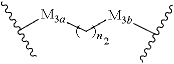

is independently selected from at least one of A2, A3 and A9; in other more specific embodiment,

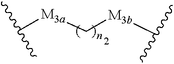

is independently selected from A3 and A9

M₃ₐ:

In one embodiment, M₃ₐ is chemical bond; in other embodiment, M₃ₐ is —O—; in other embodiment, M₃ₐ is —S—; in other embodiment, M₃ₐ is —NR₆—, such as —NH—; in other embodiment, M₃ₐ is —CHR₇—, such as —CH₂—; in other embodiment, M₃ₐ is —C(O)—; in other embodiment, M₃ₐ is —S(O)₁₋₂—; in other embodiment, M₃ₐ is —NR₆C(O)—, such as —NHC(O)—; in other embodiment, M₃ₐ is —C(O)NR₆—, such as —C(O)NH—; in other embodiment, M₃ₐ is —NR₆S(O)₁₋₂—; in other embodiment, M₃ₐ is —S(O)₁₋₂NR₆—; in other embodiment, M₃ₐ is —OC(O)—; in other embodiment, M₃ₐ is —C(O)O—; in other embodiment, M₃ₐ is —OS(O)₁₋₂—; in other embodiment, M₃ₐ is —S(O)₁₋₂O—.

In one specific embodiment, M₃ₐ is independently selected from at least one of chemical bond, —O—, —S—, —NR₆—, —CHR₇—, —C(O)—, —S(O)₁₋₂—, —NHC(O)—, —C(O)NH—, —NHS(O)₁₋₂—, —S(O)₁₋₂NH—, —OC(O)—, —C(O)O—, —OS(O)₁₋₂— and —S(O)₁₋₂O—; in other specific embodiment, M₃ₐ is independently selected from at least one of chemical bond, —O—, —S—, —NR₆—, —C(O)—, —NHC(O)—, —C(O)NH—, —OC(O)— and —C(O)O—; in other specific embodiment, M₃ₐ is independently selected from at least one of chemical bond, —NHC(O)—, —C(O)NH—, —OC(O)— and —C(O)O—; in other specific embodiment, M₃ₐ is independently selected from at least one of chemical bond, —NHC(O)— and —C(O)NH—; in other specific embodiment, M₃ₐ is independently selected from chemical bond and —C(O)NH—.

M_{3b}:

In one embodiment, M_{3b} is —O—; in other embodiment, M_{3b} is —S—; in other embodiment, M_{3b} is —NR₆—; in other embodiment, M_{3b} is —CHR₇—; in other embodiment, M_{3b} is —CH═CH—; in other embodiment, M_{3b} is —C≡C—; in other embodiment, M_{3b} is —C(O)—; in other embodiment, M_{3b} is —S(O)₁₋₂—; in other embodiment, M_{3b} is —C(O)NR₆—, such as —C(O)NH—; in other embodiment, M_{3b} is —NR₆C(O)—, such as —NHC(O)—; in other embodiment, M_{3b} is —OC(O)—; in other embodiment, M_{3b} is —C(O)O—; in other embodiment, M_{3b} is —S(O)₁₋₂O—Y₁—; in other embodiment, M_{3b} is —OS(O)₁₋₂—Y₁—; in other embodiment, M_{3b} is —OS(O)₁₋₂O—; in other embodiment, M_{3b} is —S(O)₁₋₂NR₆—Y₁—; in other embodiment, M_{3b} is —NR₆S(O)₁₋₂—Y₁—; in other embodiment, M_{3b} is C₆₋₁₀ arylene, such as phenylene; in other embodiment, M_{3b} is 5-10 membered heteroarylene, such as 5-6 membered heteroarylene, such as

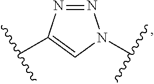 [SMILES: *c1cn(*)nn1]

such as

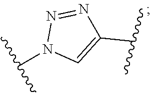 [SMILES: *c1cn(*)nn1]

in other embodiment, M_{3b} is

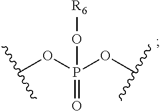

in other embodiment, M_{3b} is

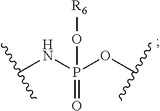

in other embodiment, M_{3b} is

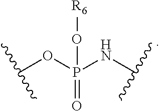

In one specific embodiment, M_{3b} is independently selected from at least one of —O—, —S—, —NR₆—, —CHR₇—, —CH═CH—, —C≡C—, —C(O)—, —S(O)₁₋₂—, —C(O)NH—, —NHC(O)—, —OC(O)—, —C(O)O—, —S(O)₁₋₂O—Y₁—, —OS(O)₁₋₂—Y₁—, —OS(O)₁₋₂O—, —S(O)₁₋₂NH—Y₁—, —NHS(O)₁₋₂—Y₁—, phenylene, 5-6 membered heteroarylene,

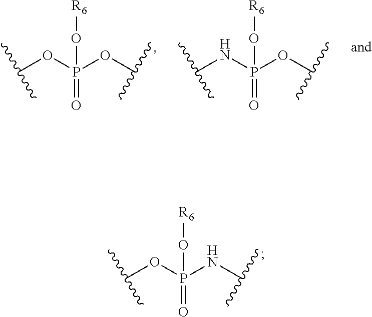

in other specific embodiment, M_{3b} is independently selected from at least one of —O—, —S—, —NR₆—, —CHR₇—, —CH═CH—, —C≡C—, —C(O)—, —C(O)NH—, —NHC(O)—, —OC(O)—, —C(O)O—, —OS(O)₁₋₂—Y₁—, —OS(O)₁₋₂O—, —NHS(O)₁₋₂—Y₁—, 5-6 membered heteroarylene,

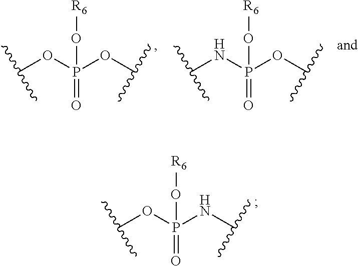

in other specific embodiment, M_{3b} is independently selected from at least one of —C(O)NH—, —NHC(O)—, —OC(O)—, and —C(O)O—; in other specific embodiment, M_{3b} is —C(O)NH— or —NHC(O)—; in other specific embodiment, M_{3b} is —NHC(O)—; in other specific embodiment, 5-6 membered heteroarylene of M_{3b} is or

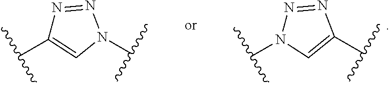 [SMILES: *c1cn(*)nn1 ; *c1cn(*)nn1]

Y₁:

In one specific embodiment, Y₁ is C₆₋₁₀ arylene, such as phenylene; in other embodiment, Y₁ is 5-10 membered heteroarylene, such as 5-6 membered heteroarylene.

In one specific embodiment, Y₁ is independently selected from phenylene or 5-6 membered heteroarylene; in other more specific embodiment, Y₁ is phenylene, such as

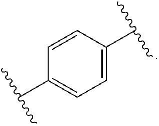 [SMILES: *c1ccc(*)cc1]

R₆:

In one specific embodiment, R₆ is H; in other embodiment, R₆ is C₁₋₁₀ alkyl, such as C₁₋₆ alkyl, such as —CH₃, such as

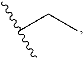 [SMILES: *CC]

such as

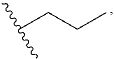 [SMILES: "," ; *CCC]

such as

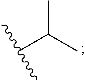 [SMILES: *C(C)C]

in other embodiment, R₆ is C₁₋₁₀ haloalkyl, such as C₁₋₆ haloalkyl; in other embodiment, R₆ is C₃₋₁₀ cycloalkyl, such as C₃₋₇ cycloalkyl; in other embodiment, R₆ is 3-10 membered heterocyclyl, such as 3-7 membered heterocyclyl.

In one more specific embodiment, R₆ is respectively and independently selected from at least one of H, C₁₋₆ alkyl, C₁₋₆ haloalkyl, C₃₋₇ cycloalkyl, 3-7 membered heterocyclyl; in other more specific embodiment, R₆ is respectively and independently selected from at least one of H, C₁₋₆ alkyl, C₁₋₆ haloalkyl; in other more specific embodiment, R₆ is respectively and independently selected from at least one of H, —CH₃,

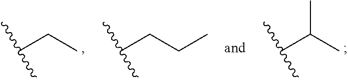

in other more specific embodiment, R₆ is H.

R₇:

In one specific embodiment, R₇ is H; in other embodiment, R₇ is halogen, such as —F, such as —Cl, such as —Br; in other embodiment, R₇ is —C≡N; in other embodiment, R₇ is —NO₂; in other embodiment, R₇ is C₁₋₁₀ alkyl; in other embodiment, R₇ is C₁₋₁₀ haloalkyl; in other embodiment, R₇ is C₃₋₁₀ cycloalkyl; in other embodiment, R₇ is 3-10 membered heterocyclyl; in other embodiment, R₇ is C₆₋₁₀ aryl; in other embodiment, R₇ is 5-10 membered heteroaryl group.

In one embodiment, one or multiple methylene of the alkyl or haloalkyl of R₇ are optionally and independently substituted by at least one of the following groups: —C(O)—, —C(O)NH—, —NHC(O)—, —OC(O)—, —O—, —C(O)O—, —S—, —S—(CH₂)₀₋₆—S—, —NR₆—, C₆₋₁₀ arylene, 5-10 membered heteroarylene, —CH═CH—, —C≡C— and —O—(CH₂)₁₋₆—O—; such as is substituted by —C(O)—, such as is substituted by —C(O)NH—, such as is substituted by —NHC(O)—, such as is substituted by —OC(O)—, such as is substituted by —O—, such as is substituted by —C(O)O—, such as is substituted by —S—, such as is substituted by —S—(CH₂)₀₋₆—S—, such as is substituted by —NR₆—, such as is substituted by C₆₋₁₀ arylene, such as is substituted by 5-10 membered heteroaryl group, such as is substituted by —CH═CH—, such as is substituted by —C≡C—, such as is substituted by —O—(CH₂)₁₋₆—O—; in other embodiment, 1, 2 or 3 methylenes of the alkyl or haloalkyl of R₇ are optionally and independently substituted by the above mentioned groups.

In one more specific embodiment, R₇ is respectively and independently selected from at least one of H, halogen, CN, C₁₋₆ alkyl, C₁₋₆ haloalkyl, C₃₋₇ cycloalkyl, 3-7 membered heterocyclyl, phenyl and 5-6 membered heteroaryl; in other more specific embodiment, R₇ is respectively and independently selected from at least one of H, halogen, CN, C₁₋₆ alkyl and C₁₋₆ haloalkyl; in other more specific embodiment, R₇ is respectively and independently selected from at least one of H, halogen, C₁₋₆ alkyl and C₁₋₆ haloalkyl.

In one more specific embodiment, one or multiple methylene of the alkyl or haloalkyl of R₇ are optionally and independently substituted by at least one of the following groups: —C(O)—, —C(O)NH—, —NHC(O)—, —OC(O)—, —O—, —C(O)O—, —S—, —S—(CH₂)₀₋₃—S—, —NR₆—, phenylene, 5-6 membered heteroarylene, —CH═CH—, —C≡C— and —O—(CH₂)₁₋₃—O—; in other more specific embodiment, 1, 2 or 3 methylenes of the alkyl or haloalkyl of R₇ are optionally and independently substituted by at least one of the following groups: —C(O)—, —C(O)NH—, —NHC(O)—, —OC(O)—, —O—, —C(O)O—, —S—, —S—S—, —NR₆—,

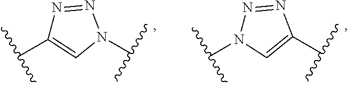 [SMILES: *c1cn(*)nn1 ; *c1cn(*)nn1]

—CH═CH—, —C≡C— and —O—(CH₂)₂—O—; in other more specific embodiment, the halogen of R₇ is —F, —Cl or —Br.

n₂:

In one more specific embodiment, n₂ is 0; in other embodiment, n₂ is 1; in other embodiment, n₂ is 2; in other embodiment, n₂ is 3; in other embodiment, n₂ is 4; in other embodiment, n₂ is 5; in other embodiment, n₂ is 6; in other embodiment, n₂ is 7; in other embodiment, n₂ is 8; in other embodiment, n₂ is 9; in other embodiment, n₂ is 10.

n₃:

In one embodiment, n₃ is 1; in other embodiment, n₃ is 2; in other embodiment, n₃ is 3; in other embodiment, n₃ is 4; in other embodiment, n₃ is 5; in other embodiment, n₃ is 6.

R₄:

In one embodiment, R₄ is H; in other embodiment, R₄ is C₁₋₆ alkyl, such as C₁₋₅ alkyl, such as C₁₋₃ alkyl; in other embodiment, R₄ is C₁₋₆ haloalkyl, such as C₁₋₅ haloalkyl, such as C₁₋₃ haloalkyl; in other embodiment, R₄ is C₁₋₆ alkoxy.

In one more specific embodiment, R₄ is selected from at least one of H, C₁₋₅ alkyl, C₁₋₅ haloalkyl and C₁₋₅ alkoxy; in other more specific embodiment, R₄ is selected from at least one of H, C₁₋₃ alkyl and C₁₋₃ haloalkyl; in other more specific embodiment, R₄ is H.

Link₁, Link₂ and Link₃:

In one embodiment, Link₁ is C₁₋₂₅ linear chain alkylene, such as C₁₋₁₀ linear chain alkylene, such as C₁₋₆ linear chain alkylene, such as C₄₋₆ linear chain alkylene, such as —(CH₂)₄—, such as —(CH₂)₆—.

In one embodiment, Link₂ is C₁₋₂₅ linear chain alkylene, such as C₁₋₁₀ linear chain alkylene, such as C₁₋₆ linear chain alkylene, such as C₄₋₆ linear chain alkylene, such as —(CH₂)₄—, such as —(CH₂)₆—.

In one embodiment, Link₃ is C₁₋₂₅ linear chain alkylene, such as C₁₋₁₀ linear chain alkylene, such as C₁₋₆ linear chain alkylene, such as C₄₋₆ linear chain alkylene, such as —(CH₂)₄—, such as —(CH₂)₆—.

In one embodiment, one or multiple methylenes of Link₁, Link₂ and Link₃ are optionally and independently substituted by R*; in other embodiment, 1, 2 or 3 methylenes of Link₁, Link₂ and Link₃ are optionally and independently substituted by R*; in other embodiment, 1 methylene of Link₁, Link₂ and Link₃ is optionally and independently substituted by R*.

In one more specific embodiment, Link₁, Link₂ and Link₃ are respectively and independently selected from C₁₋₁₀ linear chain alkylene; in other more specific embodiment, Link₁, Link₂ and Link₃ are respectively and independently selected from C₁₋₆ linear chain alkylene; in other more specific embodiment, Link₁, Link₂ and Link₃ are respectively and independently selected from C₄₋₆ linear chain alkylene; in other more specific embodiment, Link₁, Link₂ and Link₃ are respectively and independently selected from —(CH₂)₄— or —(CH₂)₆—.

In one embodiment, Link₁, Link₂ and Link₃ are respectively and independently selected from

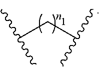

In one embodiment, n, is 1; in other embodiment, n, is 2; in other embodiment, n, is 3; in other embodiment, n, is 4; in other embodiment, n, is 5; in other embodiment, n, is 6; in other embodiment, n, is 7; in other embodiment, n, is 8; in other embodiment, n, is 9; in other embodiment, n, is 10.

Link₄:

In one embodiment, Link₄ is C₁₋₂₅ linear chain alkylene, such as C₅₋₂₅ linear chain alkylene, such as C₁₋₂₀ linear chain alkylene, such as C₅₋₂₀ linear chain alkylene, such as C₈₋₂₀ linear chain alkylene, such as C₁₋₁₇ linear chain alkylene, such as C₁₀₋₁₇ linear chain alkylene, such as C₁₋₁₀ linear chain alkylene, such as C₃₋₂₀ linear chain alkylene, such as C₃₋₁₇ linear chain alkylene, such as C₃₋₁₀ linear chain alkylene, such as —(CH₂)₃—, such as —(CH₂)₁₀—.

In one embodiment, one or multiple methylenes of Link₄ are optionally and independently substituted by R*; in other embodiment, 1, 2 or 3 methylenes of Link₄ are optionally and independently substituted by R*; in other embodiment, 1 methylene of Link₄ is optionally and independently substituted by R*, such as Link₄ is —(CH₂)₃—C(O)NH—(CH₂)₆—, such as Link₄ is —(CH₂)₁₀—C(O)NH—(CH₂)₆—; in other embodiment, the methylene connected to the carbonyl of left side in Link₄ is not substituted by R*.

In one more specific embodiment, Link₄ is selected from at least one of —(CH₂)₃—, —(CH₂)₁₀—, —(CH₂)₃—C(O)NH—(CH₂)₆— and —(CH₂)₁₀—C(O)NH—(CH₂)₆—; in other more specific embodiment, Link₄ is selected from at least one of —(CH₂)₃—, —(CH₂)₁₀— and —(CH₂)₃—C(O)NH—(CH₂)₆; in other more specific embodiment, Link₄ is —(CH₂)₃— or —(CH₂)₁₀; in one more specific embodiment, Link₄ is selected from at least one of —(CH₂)₁₀—, —(CH₂)₃—C(O)NH—(CH₂)₆— and —(CH₂)₁₀—C(O)NH—(CH₂)₆—; in other more specific embodiment, Link₄ is —(CH₂)₁₀— or —(CH₂)₁₀—C(O)NH—(CH₂)₆—.

R*:

In one embodiment, R* is —O—; in other embodiment, R* is —S—; in other embodiment, R* is —NR₆—; in other embodiment, R* is —CHR₇—; in other embodiment, R* is —C(O)—; in other embodiment, R* is —S(O)₁₋₂—; in other embodiment, R* is —NR₆C(O)—, such as —NHC(O)—; in other embodiment, R* is —C(O)NR₆—, such as —C(O)NH—; in other embodiment, R* is —NR₆S(O)₁₋₂—; in other embodiment, R* is —S(O)₁₋₂NR₆—; in other embodiment, R* is —OC(O)—; in other embodiment, R* is —C(O)O—; in other embodiment, R* is —OS(O)₁₋₂—; in other embodiment, R* is —S(O)₁₋₂O—.

In one more specific embodiment, R* is independently selected from at least one of: —O—, —NR₆—, —CHR₇—, —C(O)—, —NR₆C(O)—, —C(O)NR₆—, —OC(O)— and —C(O)O—; in other more specific embodiment, R* is independently selected from at least one of: —C(O)NH—, —NHC(O)—, —C(O)O— and —OC(O)—; in other more specific embodiment, R* is independently selected from at least one of: —C(O)NH— and —C(O)O—; in other more specific embodiment, R* is independently selected from at least one of: —C(O)NH— and —NHC(O)—; in other more specific embodiment, R* is —C(O)NH—.

In one embodiment, R* is separated from the carbonyl on the left side of Link₄ by 1-15 methylenes, such as 1 methylene, such as 2 methylenes, such as 3 methylenes, such as 4 methylenes, such as 5 methylenes, such as 6 methylenes, such as 7 methylenes, such as 8 methylenes, such as 9 methylenes, such as 10 methylenes, such as 11 methylenes, such as 12 methylenes, such as 13 methylenes, such as 14 methylenes, such as 15 methylenes.

In one more specific embodiment, R* is separated from the carbonyl on the left side of Link4 by 1-15 methylenes; in other more specific embodiment, R* is separated from the carbonyl on the left side of Link4 by 1-12 methylenes; in other more specific embodiment, R* is separated from the carbonyl on the left side of Link4 by 1-10 methylenes; in other more specific embodiment, R* is separated from the carbonyl on the left side of Link4 by 1-3 methylenes; in other more specific embodiment, R* is separated from the carbonyl on the left side of Link4 by 3-10 methylenes.

In one embodiment, R* is separated from R₂ by 1-12 methylenes, such as 1 methylene, such as 2 methylenes, such as 3 methylenes, such as 4 methylenes, such as 5 methylenes, such as 6 methylenes, such as 7 methylenes, such as 8 methylenes, such as 9 methylenes, such as 10 methylenes, such as 11 methylenes, such as 12 methylenes.

In one more specific embodiment, R* is separated from R₂ by 1-12 methylenes; in other more specific embodiment, R* is separated from R₂ by 1-10 methylenes; in other more specific embodiment, R* is separated from R₂ by 1-6 methylenes.

m₁, m₂ and m₃:

In one embodiment, m, is 1; in other embodiment, m, is 2; in other embodiment, m, is 3; in other embodiment, m, is 4; in other embodiment, m, is 5.

In one embodiment, m₂ is 1; in other embodiment, m₂ is 2; in other embodiment, m₂ is 3; in other embodiment, m₂ is 4; in other embodiment, m₂ is 5.

In one embodiment, m₃ is 1; in other embodiment, m₃ is 2; in other embodiment, m₃ is 3; in other embodiment, m₃ is 4; in other embodiment, m₃ is 5.

In one more specific embodiment, m₁, m₂ and m₃ are respectively and independently 1, 2 or 3.

This disclosure also provides a conjugate with structure shown in formula (III):

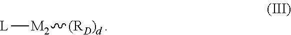

Wherein, L has structure as shown in formula (I′) or formula (II′):

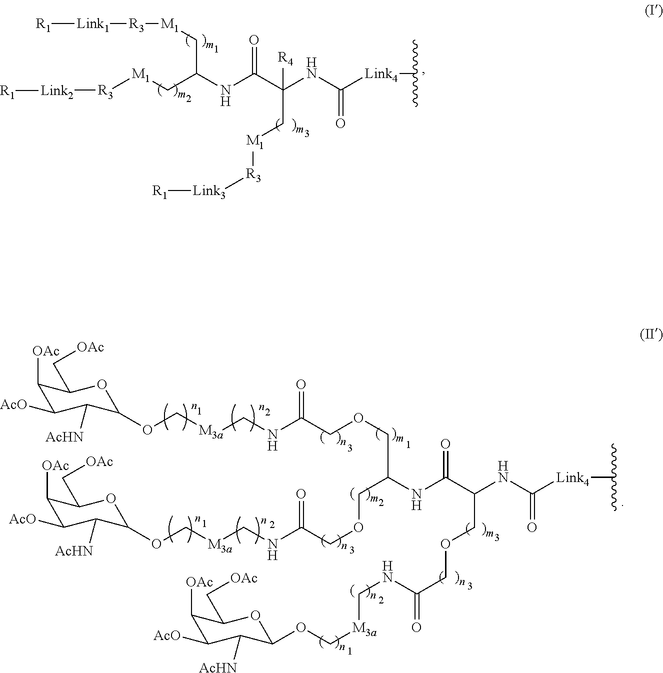

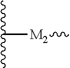

is selected from at least one of

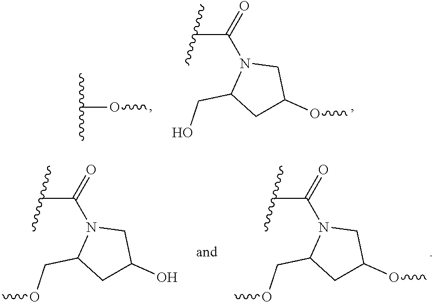

R_{D} is pharmaceutical molecule, the said pharmaceutical molecule is independently selected from at least one of cytotoxic agents, chemotherapeutic agents, somatostatin, toxins, radioisotopes and oligonucleotide strand;
d is 1 or 2, preferably 1;
each remaining groups are as defined in this disclosure.

L:

In one embodiment, L has structure as shown in formula (I′); in other embodiment, L has structure as shown in formula (II′); in other embodiment, L is L₁; in other embodiment, L is L₂; in other embodiment, L is L₃; in other embodiment, L is L₄. L₁, L₂, L₃ and L₄ are as defined in this disclosure.

In one more specific embodiment, L is selected from at least one of L₁, L₂, L₃ and L₄; in other more specific embodiment, L is selected from at least one of L₁, L₂ and L₄.

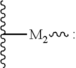

In one embodiment,

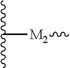

is

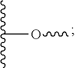

in other embodiment,

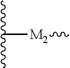

is

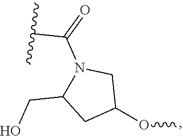

such as

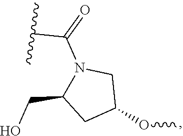

such as

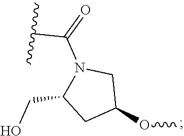

in other embodiment,

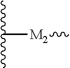

is

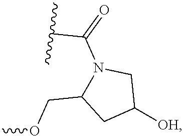

such as

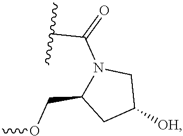

such as

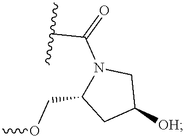

in other embodiment,

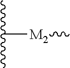

is

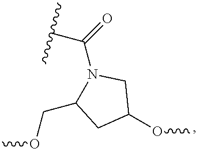

such as

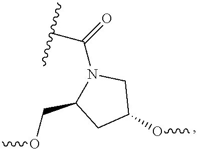

such as

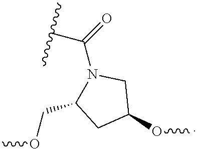

In one more specific embodiment,

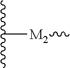

is selected from at least one of

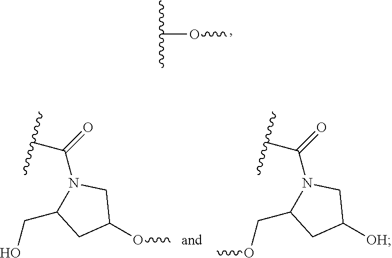

in other more specific embodiment,

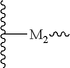

is selected from at least one of

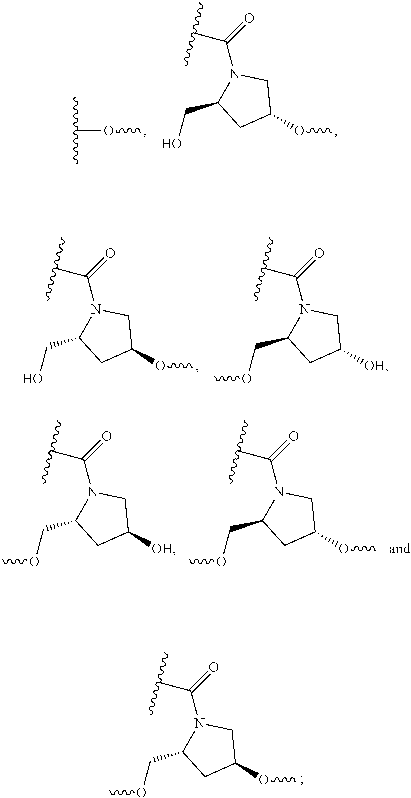

in other more specific embodiment,

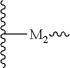

is selected from at least one of

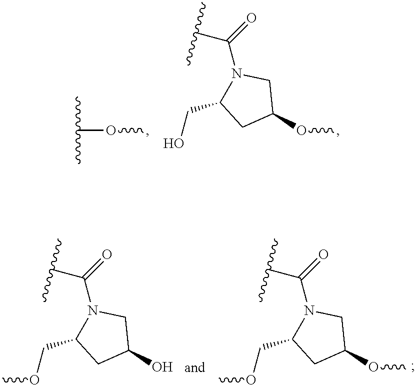

in other more specific embodiment,

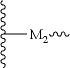

is selected from at least one of

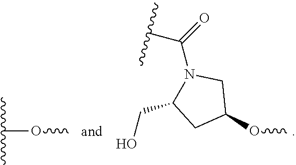

R_{D}:

In one embodiment, R_{D} is pharmaceutical molecule, such as cytotoxic agents, such as chemotherapeutic agents, such as somatostatin, such as toxins, such as radioisotopes, such as oligonucleotide strand.

In one embodiment, the oligonucleotide strand is single strand oligonucleotide; in other embodiment, the oligonucleotide strand is double strand oligonucleotide.

In one embodiment, M₂ links to 5′ end of at least one strand of the said oligonucleotide by phosphate ester bond; in other embodiment, M₂ links to 3′ end of at least one strand of the said oligonucleotide by phosphate ester bond; in other embodiment, M₂ links to any one nucleotide between 5′ end and 3′ end of at least one strand of the said oligonucleotide by phosphate ester bond; in other embodiment, M₂ links to sense strand of the said oligonucleotide by phosphate ester bond; in other embodiment, M₂ links to antisense strand of the said oligonucleotide by phosphate ester bond.

In one embodiment, the phosphate ester bond is phosphodiester bond; in other embodiment, the phosphate ester bond is modified phosphate ester bond, such as thio-modified phosphate ester bond, such as amino-modified phosphate ester bond.

d:

In one embodiment, d is 1; in other embodiment, d is 2.

Any technical solution or any combination thereof of the above specific embodiments can be combined with any technical solution or any combination thereof of other specific embodiments. For example, any technical solution or any combination thereof of M₁ can be combined with any technical solution or any combination thereof of R₁, R₂, PG₁, PG₂, Rₚ, PG₃, R, R′, R₃, M₁, M₂, M₃ₐ, M_{3b}, Y₁, R₆, R₇, n₁, n₂, n₃, R₄, R_{D}, d, Link₁, Link₂, Link₃, Link₄ and L. The present disclosure is intended to include a combination of all these technical solutions, limited to space, these technical solutions will not be listed one by one.

In more specific embodiment, this disclosure provides a compound of formula (I), or a pharmaceutical acceptable salt, tautomer or stereoisomer thereof:

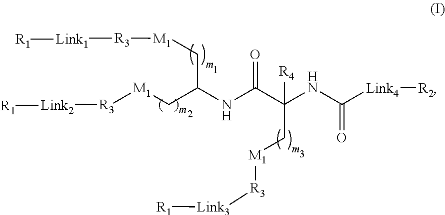

wherein,
M₁ is independently selected from at least one of —O—, —S—, —NR₆—, —CHR₇—, —C(O)—, —S(O)— and —S(O)₂—;
R₁ is residue of asialoglycoprotein receptor (ASGPR) ligand;
R₂ is selected from at least one of hydroxyl, carboxyl, —ORₚ, —OPG₂,

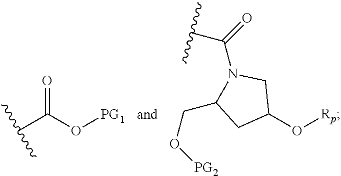

PG₂ is carboxyl protecting group;
PG₂ is hydroxyl protecting group;
Rₚ is

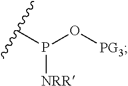

PG₃ is independently selected from phosphate protecting group;
R and R′ are respectively and independently selected from amino protecting group; or R, R′ and the N atom they linked optionally form together to be a 5-10 membered heterocyclic group;
R₃ is independently selected from

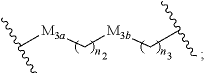

M₃ₐ is independently selected from at least one of chemical bond, —O—, —S—, —NR₆—, —CHR₇—, —C(O)—, —S(O)₁₋₂—, —NR₆C(O)—, —C(O)NR₆—, —NR₆S(O)₁₋₂—, —S(O)₁₋₂NR₆—, —OC(O)—, —C(O)O—, —OS(O)₁₋₂— and —S(O)₁₋₂O—;
M_{3b} is independently selected from at least one of —O—, —S—, —NR₆—, —CHR₇—, —CH═CH—, —C≡C—, —C(O)—, —S(O)₁₋₂—, —C(O)NR₆—, —NR₆C(O)—, —OC(O)—, —C(O)O—, —S(O)₁₋₂O—Y¹—, —OS(O)₁₋₂—Y¹—, —OS(O)₁₋₂O—, —S(O)₁₋₂NR₆—Y₁—, —NR₆S(O)₁₋₂—Y₁—, C₆₋₁₀ arylene, 5-10 membered heteroarylene,

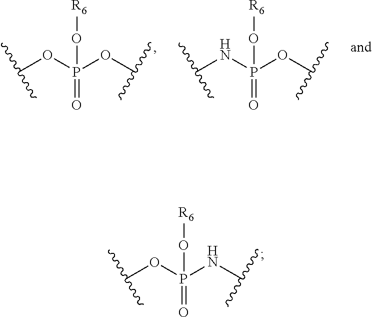

Y₁ is independently selected from C₆₋₁₀ arylene or 5-10 membered heteroarylene;
R₆ is respectively and independently selected from at least one of H, C₁₋₁₀ alkyl, C₁₋₁₀ haloalkyl, C₃₋₁₀ cycloalkyl, and 3-10 membered heterocyclyl;
R₇ is respectively and independently selected from at least one of H, halogen, —C≡N, —NO₂, C₁₋₁₀ alkyl, C₁₋₁₀ haloalkyl, C₃₋₁₀ cycloalkyl, 3-10 membered heterocyclyl, C₆₋₁₀ aryl, and 5-10 membered heteroaryl group, wherein, one or multiple methylene of the said alkyl or haloalkyl is optionally and independently substituted by the following group which is selected from at least one of: —C(O)—, —C(O)NH—, —NHC(O)—, —OC(O)—, —O—, —C(O)O—, —S—, —S—(CH₂)₀₋₆—S—, —NR₆—, C₆₋₁₀ arylene, 5-10 membered heteroarylene, —CH═CH—, —C≡C— and —O—(CH₂)₁₋₆—O—;
n₂ is independently selected from 0, 1, 2, 3, 4, 5, 6, 7, 8, 9 or 10;
n₃ is independently selected from 1, 2, 3, 4, 5 or 6;
R₄ is selected from at least one of H, C₁₋₆ alkyl, C₁₋₆ haloalkyl and C₁₋₆ alkoxy;
Link₁, Link₂, Link₃ and Link₄ are respectively and independently selected from C₁₋₂₅ linear chain alkylene, wherein, one or multiple methylene of the said linear chain alkylene is optionally and independently substituted by R*, R* is independently selected from at least one of: —O—, —S—, —NR₆—, —CHR₇—, —C(O)—, —S(O)₁₋₂—, —NR₆C(O)—, —C(O)NR₆—, —NR₆S(O)₁₋₂—, —S(O)₁₋₂NR₆—, —OC(O)—, —C(O)O—, —OS(O)₁₋₂— and —S(O)₁₋₂O—;
m₁, m₂ and m₃ are respectively and independently selected from 1, 2, 3, 4 or 5;

With the proviso that the methylene connected to the carbonyl of left side in Link₄ is not substituted by R*.

In more specific embodiment, this disclosure provides a compound of formula (I), or a pharmaceutical acceptable salt, tautomer or stereoisomer thereof, wherein M₁ is independently selected from at least one of —O—, —S—, —NH—, —CH₂—, —C(O)—, —S(O)— and —S(O)₂—, preferably selected from —O—, —S— and —NH—, preferably —O—.

In more specific embodiment, this disclosure provides a compound of formula (I), or a pharmaceutical acceptable salt, tautomer or stereoisomer thereof, wherein, R₁ is independently selected from at least one of residue of the following structure: D-mannopyranose, L-mannopyranose, L-arabinose, D-arabinose, D-xylofuranose, L-xylofuranose, D-glucose, L-glucose, D-galactose, L-galactose, α-D-mannofuranose, β-D-mannofuranose, α-D-mannopyranose, β-D-mannopyranose, α-D-glucopyranose, β-D-glucopyranose, α-D-glucofuranose, β-D-glucofuranose, α-D-fructofuranose, β-D-fructofuranose, α-D-fructopyranose, β-D-fructopyranose, α-D-galactopyranose, β-D-galactopyranose, α-D-galactofuranose, β-D-galactofuranose, glucosamine, sialic acid, galactosamine, N-acetylgalactosamine, N-trifluoroacetylgalactosamine, N-propionylgalactosamine, N-n-butyrylgalactosamine, N-isobutyrylgalactosamine, 2-amino-3-O—[(R)-1-carboxyethyl]-2-deoxy-β-D-glucopyranose, 2-deoxy-2-methylamino-L-glucopyranose, 4,6-dideoxy-4-carboxamido-2,3-di-O-methyl-D-mannopyranose, 2-deoxy-2-sulfamino-D-glucopyranose, N-glycolyl-α-neuraminic acid, 5-thio-β-D-glucopyranose, 2,3,4-tri-O-acetyl-1-thio-6-O-tribenzyl-α-D-glucopyranosyl methyl ester, 4-thio-3-D-galactopyranose, 3,4,6,7-tetra-O-acetyl-2-deoxy-1,5-dithio-α-D-glucoheptoside ethyl ester, 2,5-anhydro-D-allosenitrile, ribose, D-ribose, D-4-thioribose, L-ribose and L-4-thioribose;
preferably, R₁ is independently selected from at least one of residue of the following structure: D-galactose, L-galactose, α-D-galactopyranose, β-D-galactopyranose, α-D-galactofuranose, β-D-galactofuranose, galactosamine, N-acetylgalactosamine, N-trifluoroacetylgalactosamine, N-propionylgalactosamine, N-n-butyrylgalactosamine, N-isobutyrylgalactosamine and β-D-galactofuranose, preferably selected from at least one of: galactosamine, N-acetylgalactosamine, N-trifluoroacetylgalactosamine, N-propionylgalactosamine, N-n-butyrylgalactosamine, N-isobutyrylgalactosamine, preferably N-acetylgalactosamine;
preferably, R₁ is

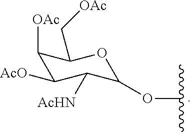 [SMILES: *OC1OC(COC(C)=O)C(OC(C)=O)C(OC(C)=O)C1NC(C)=O]

In more specific embodiment, this disclosure provides a compound of formula (I), or a pharmaceutical acceptable salt, tautomer or stereoisomer thereof, wherein, R₂ is selected from at least one of hydroxyl, carboxyl, —ORₚ, —OPG₂,

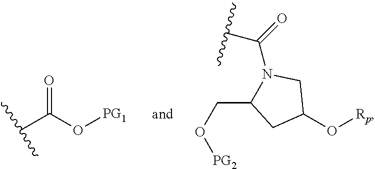

preferably selected from at least one of —ORₚ,

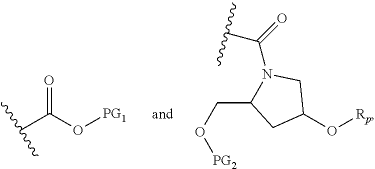

preferably selected from at least one of

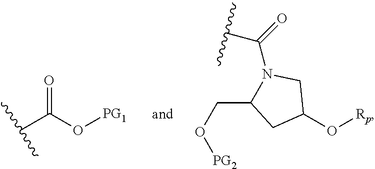

preferably selected from at least one of

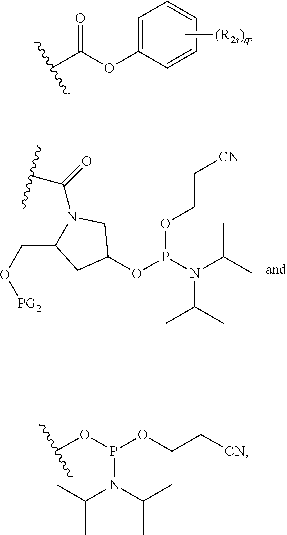

preferably selected from

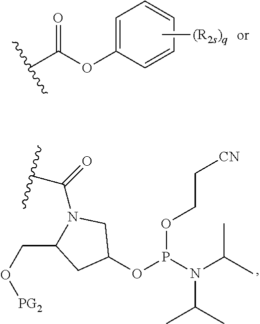

preferably

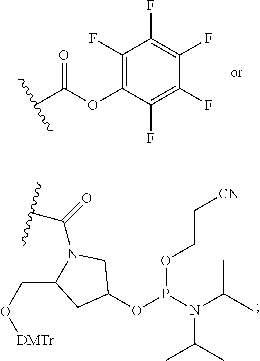

preferably

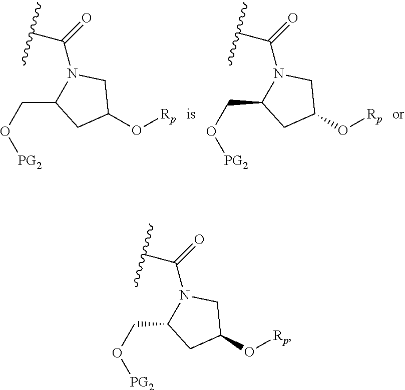

preferably

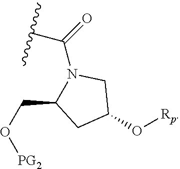

In more specific embodiment, this disclosure provides a compound of formula (I), or a pharmaceutical acceptable salt, tautomer or stereoisomer thereof, wherein, PG₁ is selected from at least one of C₁₋₆ alkyl, benzyl, allyl,

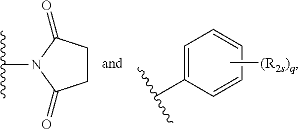

wherein, R₂ₛ is independently selected from at least one of H, halogen, C₁₋₁₀ alkyl, C₁₋₁₀ haloalkyl, q is 0, 1, 2, 3, 4 or 5;
preferably, PG₁ is selected from at least one of methyl, tert-butyl group, benzyl, allyl,

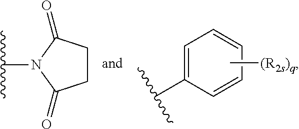

preferably selected from at least one of

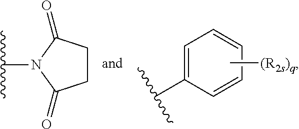

preferably

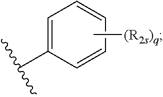

preferably, R₂ₛ is independently selected from at least one of H, halogen, C₁₋₆ alkyl and C₁₋₆ haloalkyl, preferably H or halogen, preferably —F, preferably the PG₁ is

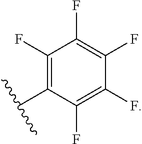 [SMILES: *c1c(F)c(F)c(F)c(F)c1F]

In more specific embodiment, this disclosure provides a compound of formula (I), or a pharmaceutical acceptable salt, tautomer or stereoisomer thereof, wherein, PG₂ is selected from at least one of: trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethyloxycarbonyl (Fmoc), allyloxycarbonyl (Alloc), 2,2,2-trichloroethoxycarbonyl (Troc), carbobenzyloxy (Cbz), tert-butyloxycarbonyl (Boc), benzyl (Bn), p-methoxybenzyl (PMB), allyl, dimethoxytrityl (DMT), monomethoxytrityl (MMT), 9-phenyloxaanthracen-9-yl (Pixyl), 9-(p-methoxyphenyl) oxaanthracen-9-yl (Mox), trityl (Tr), 4-methoxytriphenylmethyl (MMTr), 4,4′-dimethoxytrityl (DMTr), 4,4′,4″-trimethoxytriphenylmethyl (TMTr), methoxymethyl (MOM), phenoxymethyl (BOM), 2,2,2-trichloroethoxymethyl, 2-methoxyethoxymethyl (MEM), methylthiomethyl (MTM), and p-methoxybenzyloxymethyl (PMBM);
preferably, PG₂ is selected from at least one of dimethoxytrityl (DMT), monomethoxytrityl (MMT), 9-phenyloxaanthracen-9-yl (Pixyl), 9-(p-methoxyphenyl) oxaanthracen-9-yl (Mox), trityl (Tr), 4-methoxytriphenylmethyl (MMTr), 4,4′-dimethoxytrityl (DMTr), 4,4′,4″-trimethoxytriphenylmethyl (TMTr), tert-butyldimethylsilyl (TBDMS), tert-butyldiphenylsilyl (TBDPS), triisopropylsilyl (TIPS), preferably selected from at least one of dimethoxytrityl (DMT), monomethoxytrityl (MMT), trityl (Tr), 4-methoxytriphenylmethyl (MMTr), 4,4′-dimethoxytrityl (DMTr), and 4,4′,4″-trimethoxytriphenylmethyl (TMTr), preferably 4,4′-dimethoxytrityl (DMTr).

In more specific embodiment, this disclosure provides a compound of formula (I), or a pharmaceutical acceptable salt, tautomer or stereoisomer thereof, wherein, PG₃ is independently selected from at least one of 2-cyanoethyl and C₁₋₆ alkyl, preferably selected from at least one of 2-cyanoethyl and methyl, preferably 2-cyanoethyl.

In more specific embodiment, this disclosure provides a compound of formula (I), or a pharmaceutical acceptable salt, tautomer or stereoisomer thereof, wherein, R and R′ are respectively and independently selected from C₁₋₆ alkyl, preferably isopropyl.

In more specific embodiment, this disclosure provides a compound of formula (I), or a pharmaceutical acceptable salt, tautomer or stereoisomer thereof, wherein, R, R′ and the N atom they linked optionally form together to be a 5-6 membered heterocyclic group.

In more specific embodiment, this disclosure provides a compound of formula (I), or a pharmaceutical acceptable salt, tautomer or stereoisomer thereof, wherein, Rₚ is

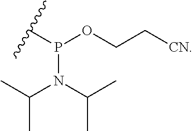 [SMILES: *P(OCCC#N)N(C(C)C)C(C)C]

In more specific embodiment, this disclosure provides a compound of formula (I), or a pharmaceutical acceptable salt, tautomer or stereoisomer thereof, wherein, M₃ₐ is independently selected from at least one of chemical bond, —O—, —S—, —NR₆—, —CHR₇—, —C(O)—, —S(O)₁₋₂—, —NHC(O)—, —C(O)NH—, —NHS(O)₁₋₂—, —S(O)₁₋₂NH—, —OC(O)—, —C(O)O—, —OS(O)₁₋₂— and —S(O)₁₋₂O—, preferably selected from at least one of chemical bond, —O—, —S—, —NR₆—, —C(O)—, —NHC(O)—, —C(O)NH—, —OC(O)— and —C(O)O—, preferably selected from at least one of chemical bond, —NHC(O)—, —C(O)NH—, —OC(O)— and —C(O)O—, preferably selected from at least one of chemical bond, —NHC(O)— and —C(O)NH—, preferably chemical bond or —C(O)NH—.

In more specific embodiment, this disclosure provides a compound of formula (I), or a pharmaceutical acceptable salt, tautomer or stereoisomer thereof, wherein, M_{3b} is independently selected from at least one of —O—, —S—, —NR₆—, —CHR₇—, —CH═CH—, —C≡C—, —C(O)—, —S(O)₁₋₂—, —C(O)NH—, —NHC(O)—, —OC(O)—, —C(O)O—, —S(O)₁₋₂O—Y₁—, —OS(O)₁₋₂—Y₁—, —OS(O)₁₋₂O—, —S(O)₁₋₂NH—Y₁—, —NHS(O)₁₋₂—Y₁—, phenylene, 5-6 membered heteroarylene,

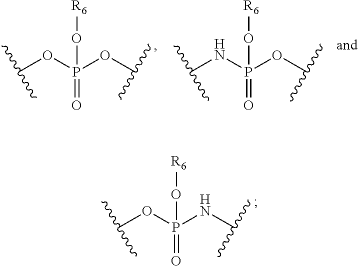

preferably, M_{3b} is independently selected from at least one of —O—, —S—, —NR₆—, —CHR₇—, —CH═CH—, —C≡C—, —C(O)—, —C(O)NH—, —NHC(O)—, —OC(O)—, —C(O)O—, —OS(O)₂—Y₁—, —OS(O)₂O—, —NHS(O)₂—Y₁—, 5-6 membered heteroarylene,

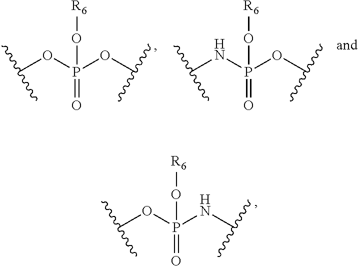

preferably selected from at least one of —C(O)NH—, —NHC(O)—, —OC(O)—, —C(O)O— preferably —C(O)NH— or —NHC(O)—, preferably —NHC(O)—;
preferably, the 5-6 membered heteroarylene of M_{3b} is

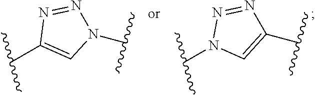 [SMILES: *c1cn(*)nn1 ; *c1cn(*)nn1]

preferably, Y₁ is independently selected from phenylene, 5-6 membered heteroarylene, preferably phenylene, preferably

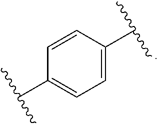 [SMILES: *c1ccc(*)cc1]

In more specific embodiment, this disclosure provides a compound of formula (I), or a pharmaceutical acceptable salt, tautomer or stereoisomer thereof, wherein,

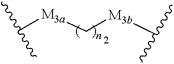

is selected from at least one of the following structure:

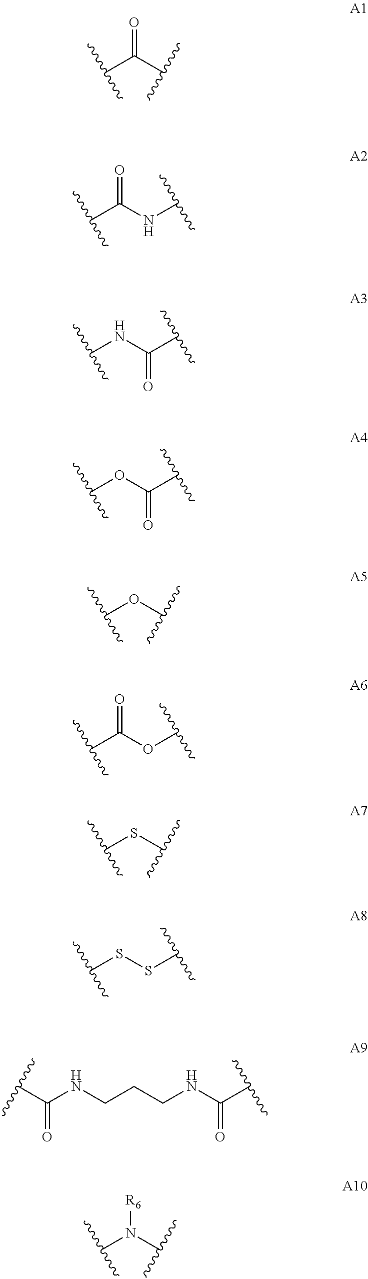

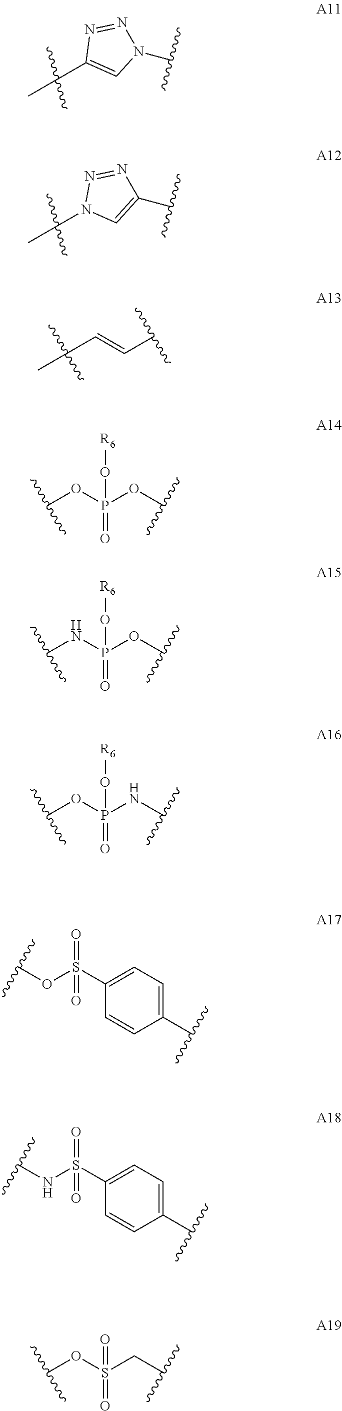

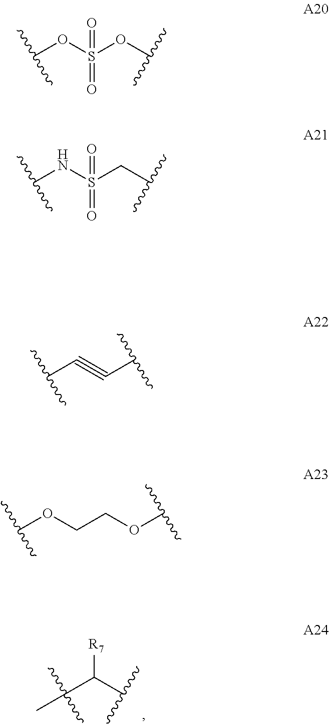

preferably selected from at least one of A2, A3, A4, A6 and A9, preferably selected from at least one of A2, A3, A9, preferably A3 or A9.

In more specific embodiment, this disclosure provides a compound of formula (I), or a pharmaceutical acceptable salt, tautomer or stereoisomer thereof, wherein, R₆ is independently selected from at least one of H, C₁₋₆ alkyl, C₁₋₆ haloalkyl, C₃₋₇ cycloalkyl and 3-7 membered heterocyclyl, preferably selected from at least one of H, C₁₋₆ alkyl, C₁₋₆ haloalkyl preferably selected from H, —CH₃,

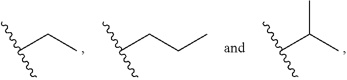

more preferably H.

In more specific embodiment, this disclosure provides a compound of formula (I), or a pharmaceutical acceptable salt, tautomer or stereoisomer thereof, wherein, R₇ is independently selected from at least one of H, halogen, —C≡N, C₁₋₆ alkyl, C₁₋₆ haloalkyl, C₃₋₇ cycloalkyl, 3-7 membered heterocyclyl, phenyl, 5-6 membered heteroaryl, preferably selected from at least one of H, halogen, —C≡N, C₁₋₆ alkyl and C₁₋₆ haloalkyl, preferably selected from at least one of H, halogen, C₁₋₆ alkyl and C₁₋₆ haloalkyl;
preferably, one or multiple methylene of the said alkyl or haloalkyl of R₇ are optionally and independently substituted by the following groups which are selected from at least one of: —C(O)—, —C(O)NH—, —NHC(O)—, —OC(O)—, —O—, —C(O)O—, —S—, —S—(CH₂)₀₋₃—S—, —NR₆—, phenylene, 5-6 membered heteroarylene, —CH═CH—, —C≡C— and —O—(CH₂)₁₋₃—O—, preferably selected from at least one of —C(O)—, —C(O)NH—, —NHC(O)—, —OC(O)—, —O—, —C(O)O—, —S—, —S—S—, —NR₆—,

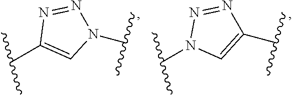

—CH═CH—, —C≡C— and —O—(CH₂)₂—O—;
preferably, the halogen of R₇ is —F, —Cl or —Br.

In more specific embodiment, this disclosure provides a compound of formula (I), or a pharmaceutical acceptable salt, tautomer or stereoisomer thereof, wherein, n₂ is independently 0, 1, 2, 3, 4, 5 or 6, preferably 0, 1, 2 or 3, preferably 0 or 3.

In more specific embodiment, this disclosure provides a compound of formula (I), or a pharmaceutical acceptable salt, tautomer or stereoisomer thereof, wherein, n₃ is independently 1, 2, 3, preferably 2.

In more specific embodiment, this disclosure provides a compound of formula (I), or a pharmaceutical acceptable salt, tautomer or stereoisomer thereof, wherein, R₄ is selected from at least one of H, C₁₋₅ alkyl, C₁₋₅ haloalkyl and C₁₋₅ alkoxy, preferably selected from at least one of H, C₁₋₃ alkyl and C₁₋₃ haloalkyl, preferably is H.

In more specific embodiment, this disclosure provides a compound of formula (I), or a pharmaceutical acceptable salt, tautomer or stereoisomer thereof, wherein, Link₁, Link₂ and Link₃ are respectively and independently selected from C₁₋₁₀ linear chain alkylene, preferably C₁₋₆ linear chain alkylene, preferably C₄₋₆ linear chain alkylene, preferably —(CH₂)₄— or —(CH₂)₆—.

In more specific embodiment, this disclosure provides a compound of formula (I), or a pharmaceutical acceptable salt, tautomer or stereoisomer thereof, wherein, Link₄ is C₁₋₂₀ linear chain alkylene, preferably C₁₋₁₇ linear chain alkylene, preferably C₁₋₁₀ linear chain alkylene, preferably C₃₋₂₀ linear chain alkylene, preferably C₃₋₁₇ linear chain alkylene, preferably C₃₋₁₀ linear chain alkylene;
preferably, one or multiple methylenes of the linear chain alkylene of Link₄, preferably 1, 2, or 3 methylenes, preferably 1 methylene is optionally and independently substituted by R*;
preferably, R* is independently selected from at least one of: —O—, —NR₆—, —CHR₇—, —C(O)—, —NR₆C(O)—, —C(O)NR₆—, —OC(O)— and —C(O)O—, preferably at least one of —C(O)NH—, —NHC(O)—, —C(O)O— and —OC(O)—, preferably at least one of —C(O)NH— and —C(O)O—, preferably at least one of —C(O)NH— and —NHC(O)—, preferably —C(O)NH—;
preferably, R* is separated from the carbonyl on the left side of Link₄ by 1-15 methylenes, preferably 1-12 methylenes, preferably 1-10 methylenes, preferably 1-3 methylenes, preferably 3-10 methylenes;
R is separated from R₂ by 1-12 methylenes, preferably 1-10 methylenes, preferably 1-6 methylenes;
preferably, Link₄ is selected from at least one of —(CH₂)₃—, —(CH₂)₁₀—, —(CH₂)₃—C(O)NH—(CH₂)₆— and —(CH₂)₁₀—C(O)NH—(CH₂)₆—, preferably selected from at least one of —(CH₂)₃—, —(CH₂)₁₀— and —(CH₂)₃—C(O)NH—(CH₂)₆—, preferably —(CH₂)₃— or —(CH₂)₁₀—, or preferably the Link₄ is selected from at least one of —(CH₂)₁₀—, —(CH₂)₃—C(O)NH—(CH₂)₆— and —(CH₂)₁₀—C(O)NH—(CH₂)₆—, preferably —(CH₂)₁₀— or —(CH₂)₁₀—C(O)NH—(CH₂)₆—.

In more specific embodiment, this disclosure provides a compound of formula (I), or a pharmaceutical acceptable salt, tautomer or stereoisomer thereof, wherein, m₁, m₂ and m₃ are respectively and independently 1, 2 or 3, preferably 1.

In more specific embodiment, this disclosure provides a compound of formula (I), or a pharmaceutical acceptable salt, tautomer or stereoisomer thereof, which have the following structures:

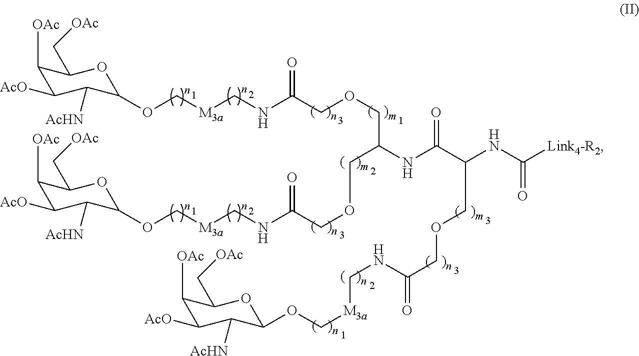

wherein,
n₂ is independently 0, 1, 2, 3, 4, 5, 6, 7, 8, 9 or 10;
each remaining groups are as defined in this disclosure.

In more specific embodiment, this disclosure provides a compound of formula (II), or a pharmaceutical acceptable salt, tautomer or stereoisomer thereof, wherein, R₂ is selected from at least one of hydroxyl, carboxyl, —ORₚ, —OPG₂,

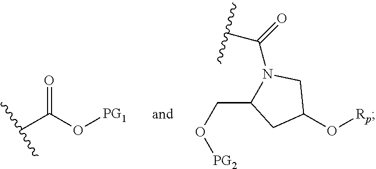

PG₁ is carboxyl protecting group, preferably selected from at least one of methyl, tert-butyl group, benzyl, allyl,

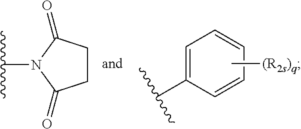

R₂ₛ is independently selected from at least one of H, halogen, C₁₋₆ alkyl and C₁₋₆ haloalkyl;
q is 0, 1, 2, 3, 4 or 5;
PG₂ is hydroxyl protecting group, preferably selected from at least one of dimethoxytrityl (DMT), monomethoxytrityl (MMT), 9-phenyloxaanthracen-9-yl (Pixyl), 9-(p-methoxyphenyl) oxaanthracen-9-yl (Mox), trityl (Tr), 4-methoxytriphenylmethyl (MMTr), 4,4′-dimethoxytrityl (DMTr), 4,4′,4″-trimethoxytriphenylmethyl (TMTr), tert-butyldimethylsilyl (TBDMS), tert-butyldiphenylsilyl (TBDPS), triisopropylsilyl (TIPS);
Rₚ is

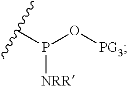

PG₃ is independently selected from phosphate protecting group, preferably selected from 2-cyanoethyl and methyl, more preferably 2-cyanoethyl;
R and R′ are respectively and independently amino protecting group, preferably C₁₋₆ alkyl, preferably isopropyl;
M₃ₐ is selected from at least one of chemical bond, —NHC(O)—, —C(O)NH—, —OC(O)— and —C(O)O—, preferably selected from at least one of chemical bond, —NHC(O)—, —C(O)NH—;
n₁ is independently 1, 2, 3, 4, 5, 6, 7, 8, 9 or 10;
n₂ is independently 0, 1, 2, 3, 4, 5 or 6;
n₃ is independently 1, 2, 3, 4, 5 or 6;
m₁, m₂ and m₃ are independently 1, 2, 3, 4 or 5;
Link₄ is selected from C₁₋₂₅ linear chain alkylene, preferably C₁₋₂₀ linear chain alkylene, wherein, 1, 2 or 3 methylenes of the said linear chain alkylene are optionally and independently substituted by R*, R* is independently selected from at least one of: —C(O)NH—, —NHC(O)—, —C(O)O— and —OC(O)—, preferably at least one of —C(O)NH— and —NHC(O)—;
with the proviso that the methylene connected to the carbonyl of left side in Link₄ is not substituted by R*.

In more specific embodiment, this disclosure provides a compound of formula (II), or a pharmaceutical acceptable salt, tautomer or stereoisomer thereof, wherein, R₂ is selected from at least one of —ORₚ,

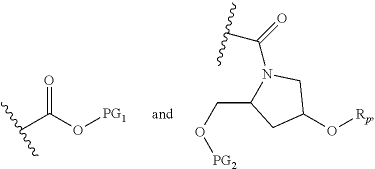

preferably selected from at least one of

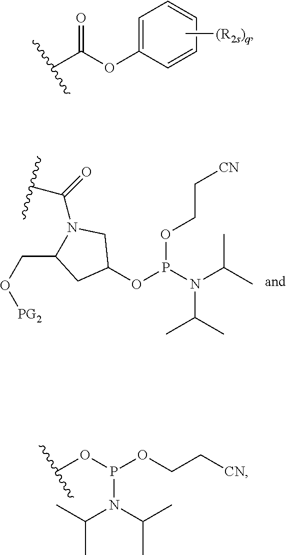

preferably

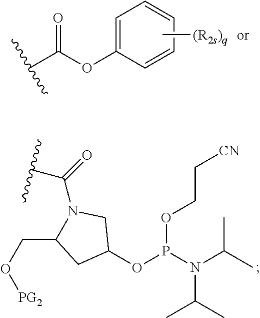

PG₁ is selected from at least one of

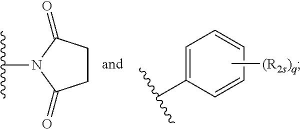

R₂ₛ is independently H or halogen, preferably —F;
q is 0, 1, 2, 3, 4 or 5;
PG₂ is selected from at least one of dimethoxytrityl (DMT), monomethoxytrityl (MMT), trityl (Tr), 4-methoxytriphenylmethyl (MMTr), 4,4′-dimethoxytrityl (DMTr), 4,4′,4″-trimethoxytriphenylmethyl (TMTr), preferably 4,4′-dimethoxytrityl (DMTr);
Rₚ is

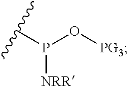

PG₃ is 2-cyanoethyl;
R and R′ are respectively and independently C₁₋₄ alkyl, preferably isopropyl;
M₃ₐ is chemical bond or —C(O)NH—;
n₁ is independently 4, 5 or 6;
n₂ is independently 0, 1, 2 or 3;
n₃ is independently 1 or 2;
m₁, m₂ and m₃ are respectively and independently 1, 2 or 3, preferably 1;
Link₄ is selected from C₁₋₁₇ linear chain alkylene, preferably C₁₋₁₀ linear chain alkylene, wherein, one methylene of the said linear chain alkylene is optionally substituted by R*, R* is —C(O)NH—;
preferably, R* is separated from the carbonyl on the left side of Link₄ by 1-12 methylenes, preferably 1-10 methylenes, preferably 1-3 methylenes;
R* is separated from R₂ by 1-12 methylenes, preferably 1-10 methylenes, preferably 1-6 methylenes.

In more specific embodiment, this disclosure provides a compound of formula (II), or a pharmaceutical acceptable salt, tautomer or stereoisomer thereof, wherein, R₂ is selected from at least one of

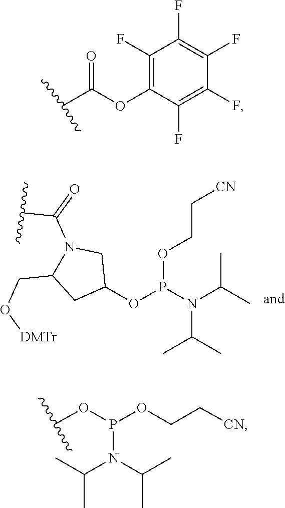

preferably

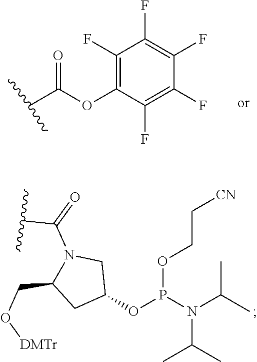

M₃ₐ is chemical bond or —C(O)NH—;
n₁ is independently 4 or 6;
n₂ is independently 0 or 3;
n₃ is 2;
m₁, m₂ and m₃ are all 1;
Link₄ is selected from —(CH₂)₃—, —(CH₂)₁₀—, —(CH₂)₃—C(O)NH—(CH₂)₆— or —(CH₂)₁₀—C(O)NH—(CH₂)₆—, preferably at least one of —(CH₂)₃—, —(CH₂)₁₀— and —(CH₂)₃—C(O)NH—(CH₂)₆—, preferably —(CH₂)₃— or —(CH₂)₁₀—.

In more specific embodiment, this disclosure provides a compound of formula (II), or a pharmaceutical acceptable salt, tautomer or stereoisomer thereof, wherein, the said compound is selected from at least one of the following:

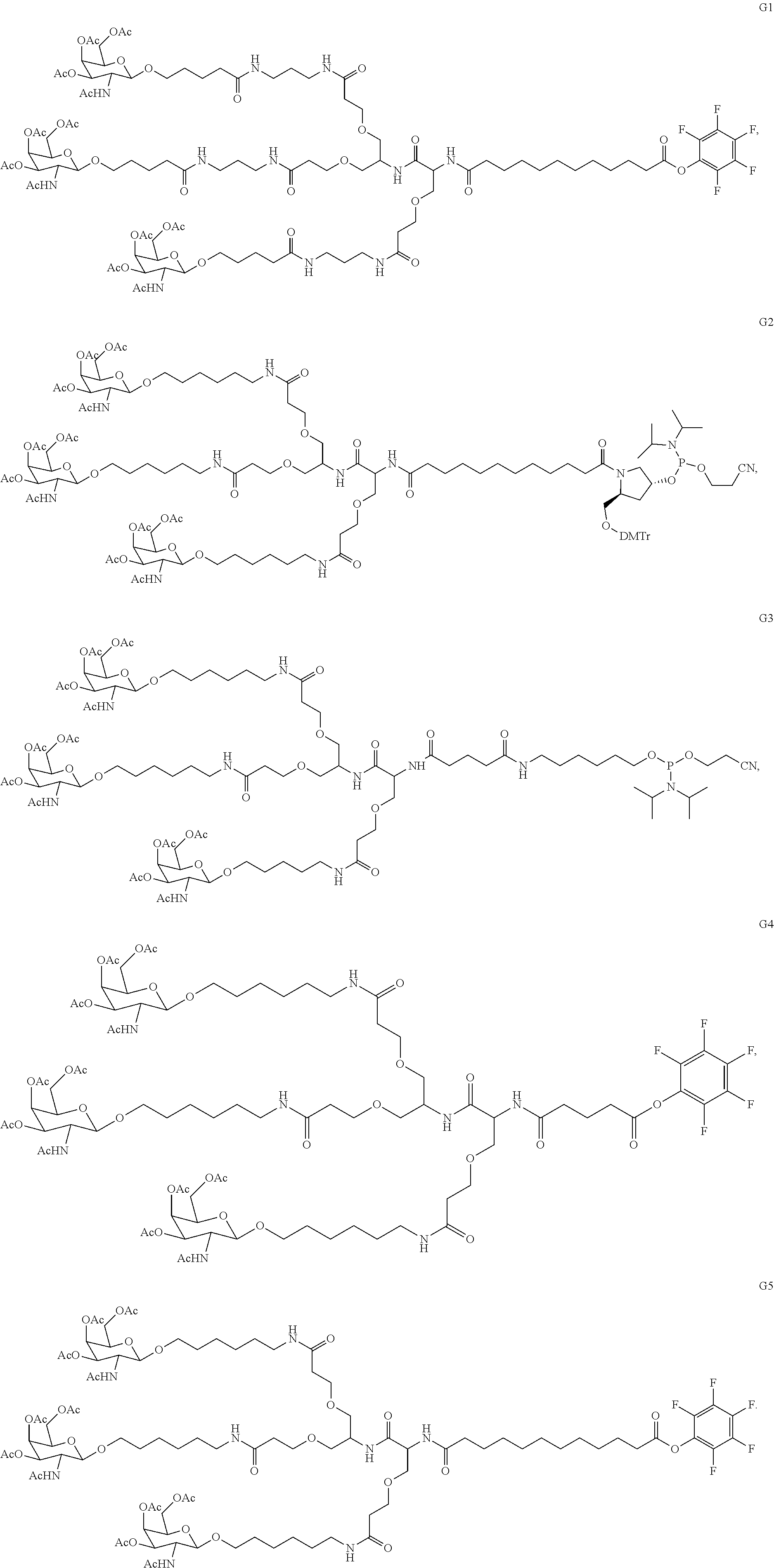

In more specific embodiment, this disclosure provides a conjugate, which has the structure as shown in formula (III):

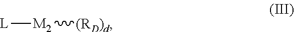

wherein,
L has structure as shown in formula (I′) or formula (II′):

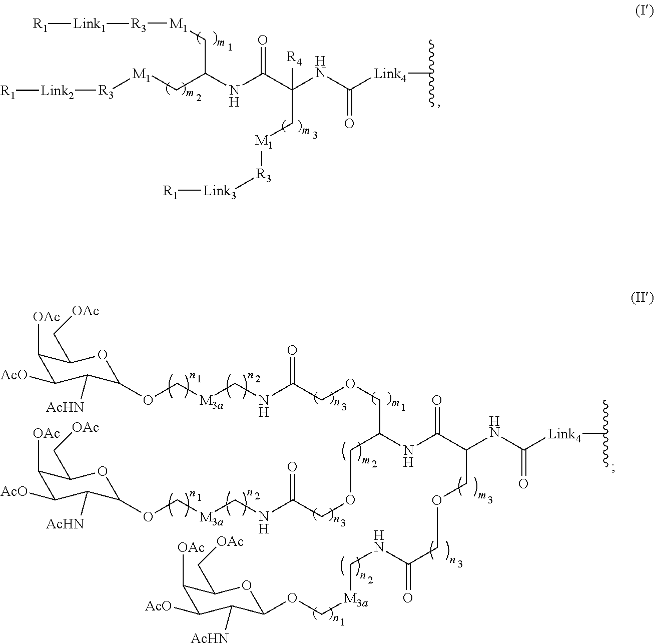

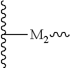

is selected from at least one of

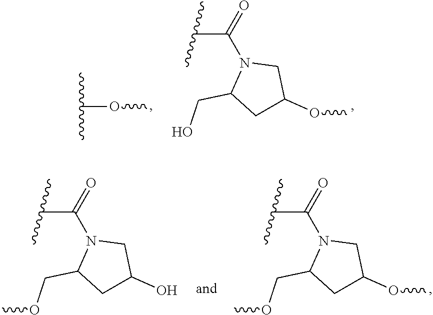

preferably selected from at least one of

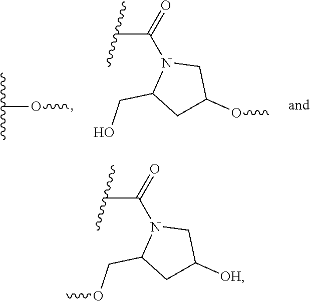

preferably selected from at least one of

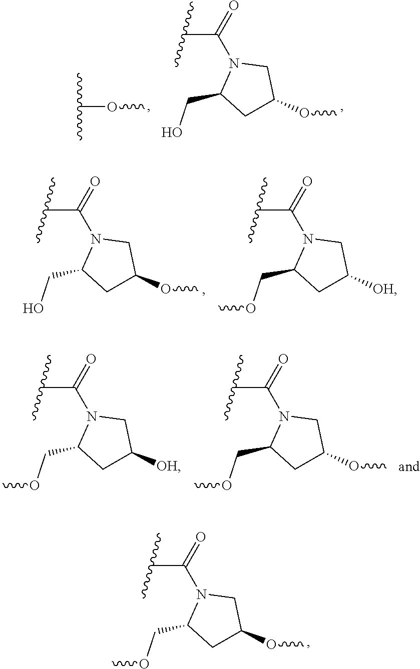

preferably selected from at least one of

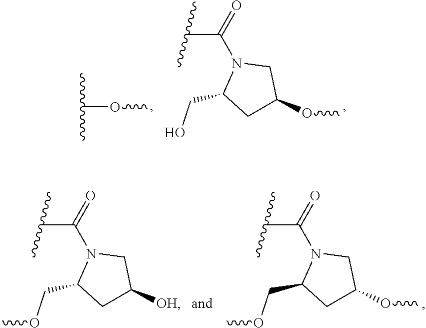

preferably selected from at least one of

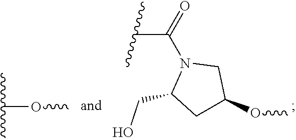

R_{D} is pharmaceutical molecule, the said pharmaceutical molecule is independently selected from cytotoxic agents, chemotherapeutic agents, somatostatin, toxins, radioisotopes and oligonucleotide strand;
d is 1 or 2, preferably 1;
each remaining groups are as defined in this disclosure;
preferably, Link₄ is selected from C₅₋₂₅ linear chain alkylene, preferably C₅₋₂₀ linear chain alkylene, preferably C₈₋₂₀ linear chain alkylene, preferably C₁₀₋₁₇ linear chain alkylene;
preferably, one or multiple methylenes of Link₄, preferably 1, 2 or 3 methylenes, preferably 1 methylene is optionally and independently substituted by R*, R* is independently selected from at least one of: —C(O)NH—, —NHC(O)—, —C(O)O— and —OC(O)—, preferably at least one of —C(O)NH— and —C(O)O—, preferably at least one of —C(O)NH— and —NHC(O)—, preferably —C(O)NH—;
preferably, R* is separated from the carbonyl on the left side of Link₄ by 1-15 methylenes, preferably 1-12 methylenes, preferably 1-10 methylenes, preferably 3-10 methylenes;
R* is separated from R₂ by 1-12 methylenes, preferably 1-10 methylenes, preferably 1-6 methylenes;
preferably, Link₄ is selected from at least one of —(CH₂)₁₀—, —(CH₂)₃—C(O)NH—(CH₂)₆— and —(CH₂)₁₀—C(O)NH—(CH₂)₆—, preferably —(CH₂)₁₀— or —(CH₂)₁₀—C(O)NH—(CH₂)₆—.

In a more specific embodiment, the present disclosure provides a conjugate of formula (III) as described above, or a pharmaceutically acceptable salt, tautomer, or stereoisomer thereof, wherein, R_{D} is an oligonucleotide chain, optionally, the oligonucleotide chain is a single-stranded oligonucleotide or a double-stranded oligonucleotide, or a composition thereof.

Preferably, M₂ is linked via a phosphate linkage to any of the nucleotides between the 5-terminus, the 3-terminus or the 5′-terminus and the 3-terminus of at least one strand of said oligonucleotide chain, preferably to the 5′-terminus or the 3-terminus of at least one strand, preferably to the 5′-terminus of at least one strand, preferably to the 3-terminus of at least one strand.

Preferably, M₂ is linked to the sense strand of said oligonucleotide chain via a phosphate linkage.

Preferably, the phosphate bond is a phosphodiester bond or a modified phosphate bond, preferably a phosphodiester bond; preferably, the modified phosphate bond is selected from the group consisting of thio-modified phosphate bonds and/or amino-modified phosphate bonds, preferably thio-modified phosphate bonds.

Preferably, the structure

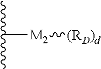

is selected from at least one of the following structures:

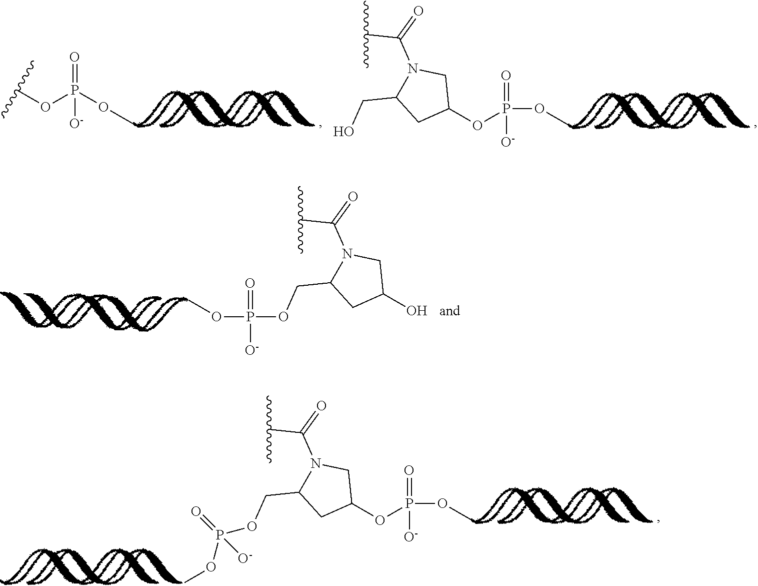

preferably from at least one of

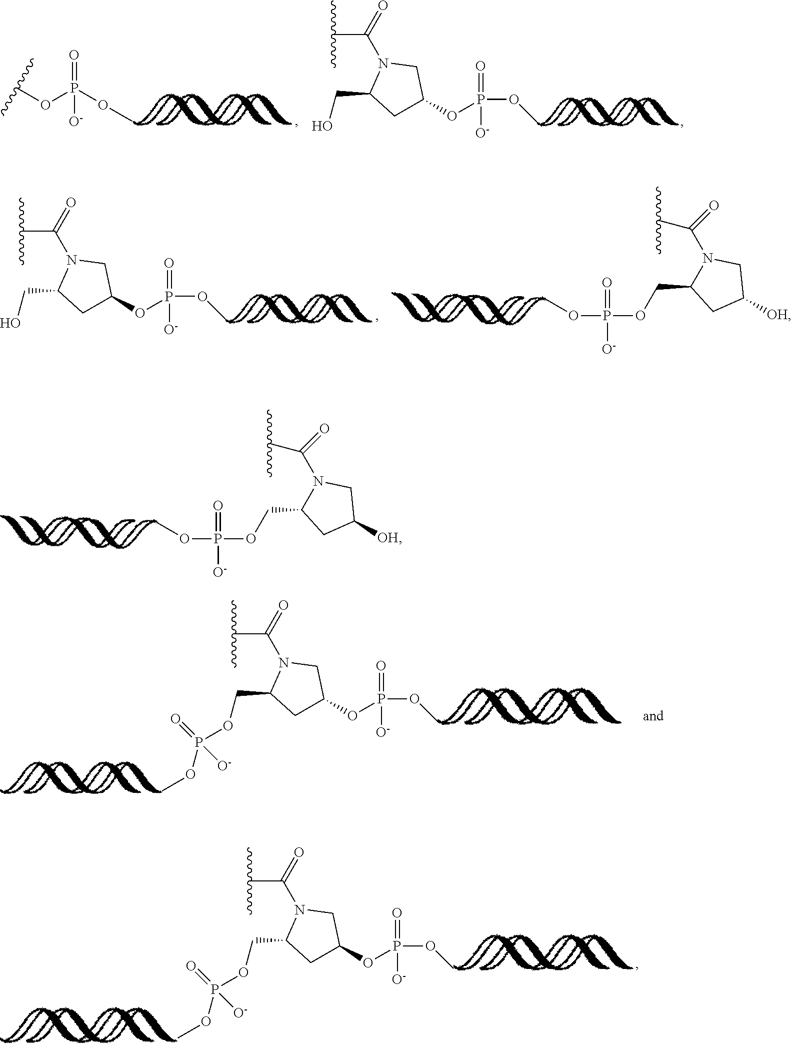

preferably from at least one of

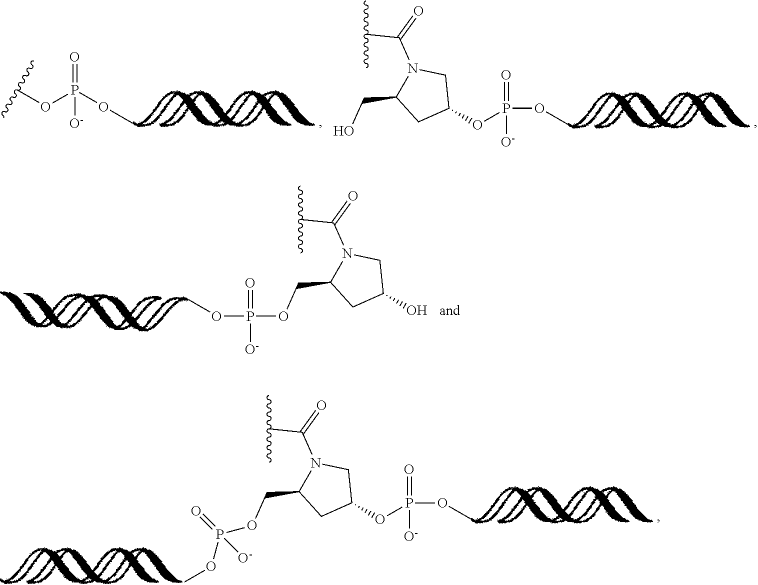

wherein, [symbol] denotes oligonucleotide chain.

In more specific embodiment, this disclosure provides a conjugate of formula (III), or a pharmaceutical acceptable salt, tautomer or stereoisomer thereof, wherein, the said oligonucleotide strand includes unmodified nucleotide and/or modified nucleotide;
preferably, the said modified nucleotide is respectively and independently selected from at least one of 2′-methoxyethyl modified nucleotide, 2′-O-alkyl modified nucleotide, 2′-O-allyl modified nucleotide, 2′-C-allyl modified nucleotide, 2′-fluoro modified nucleotide, 2′-deoxy modified nucleotide, 2′-hydroxy modified nucleotide, thiophosphoryl backbone modified nucleotide, locked nucleotide modified nucleotide, glycol nucleic acid (GNA) modified nucleotide and unlocked nucleic acid (UNA) modified nucleotide, preferably selected from at least one of 2′—O-alkyl modified nucleotide, 2′-fluoro modified nucleotide, 2′-deoxy modified nucleotide and thiophosphoryl backbone modified nucleotide;
preferably, the said 2′-O-alkyl is 2′-O—C₁₋₆ alkyl, preferably 2′-O-methyl;
preferably, the length of the said oligonucleotide strand is 5-100 bp.

In more specific embodiment, this disclosure provides a conjugate of formula (III), or a pharmaceutical acceptable salt, tautomer or stereoisomer thereof, wherein, the said oligonucleotide strand is siRNA, ASO or microRNA, preferably siRNA.

In more specific embodiment, this disclosure provides a conjugate of formula (III), or a pharmaceutical acceptable salt, tautomer or stereoisomer thereof, wherein, the said oligonucleotide strand has terminal modifier, the said terminal modifier is selected from cholesterol, polyethylene glycol, fluorescent probe, biotin, polypeptide, vitamin, or tissue targeting molecule, or the composition thereof.

In more specific embodiment, this disclosure provides a conjugate of formula (III), or a pharmaceutical acceptable salt, tautomer or stereoisomer thereof, wherein, the ribose-phosphate backbone of the said oligonucleotide strand is substituted by polypeptide nucleic acid (PNA) or morpholine ring antisense nucleotide (PMO).

In more specific embodiment, this disclosure provides a conjugate of formula (III), or a pharmaceutical acceptable salt, tautomer or stereoisomer thereof, wherein, L is selected from at least one of the following structure:

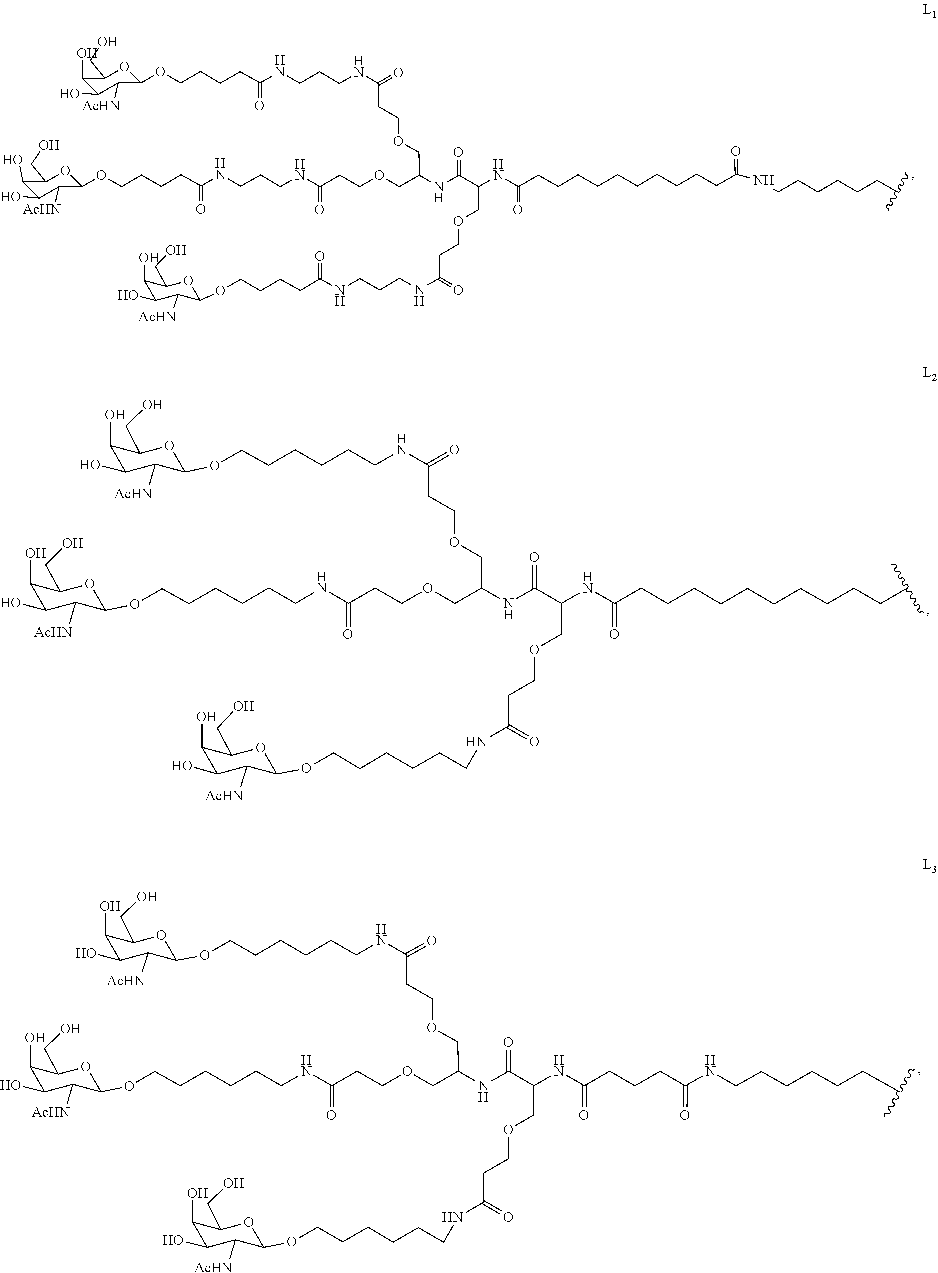

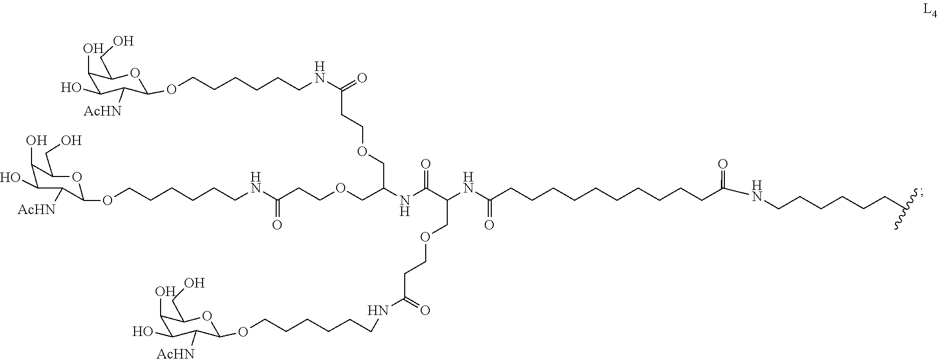 [SMILES: *CCCCCCNC(=O)CCCCCCCCCCC(=O)NC(COCCC(=O)NCCCCCCOC1OC(CO)C(O)C(O)C1NC(C)=O)C(=O)NC(COCCC(=O)NCCCCCCOC1OC(CO)C(O)C(O)C1NC(C)=O)COCCC(=O)NCCCCCCOC1OC(CO)C(O)C(O)C1NC(C)=O]

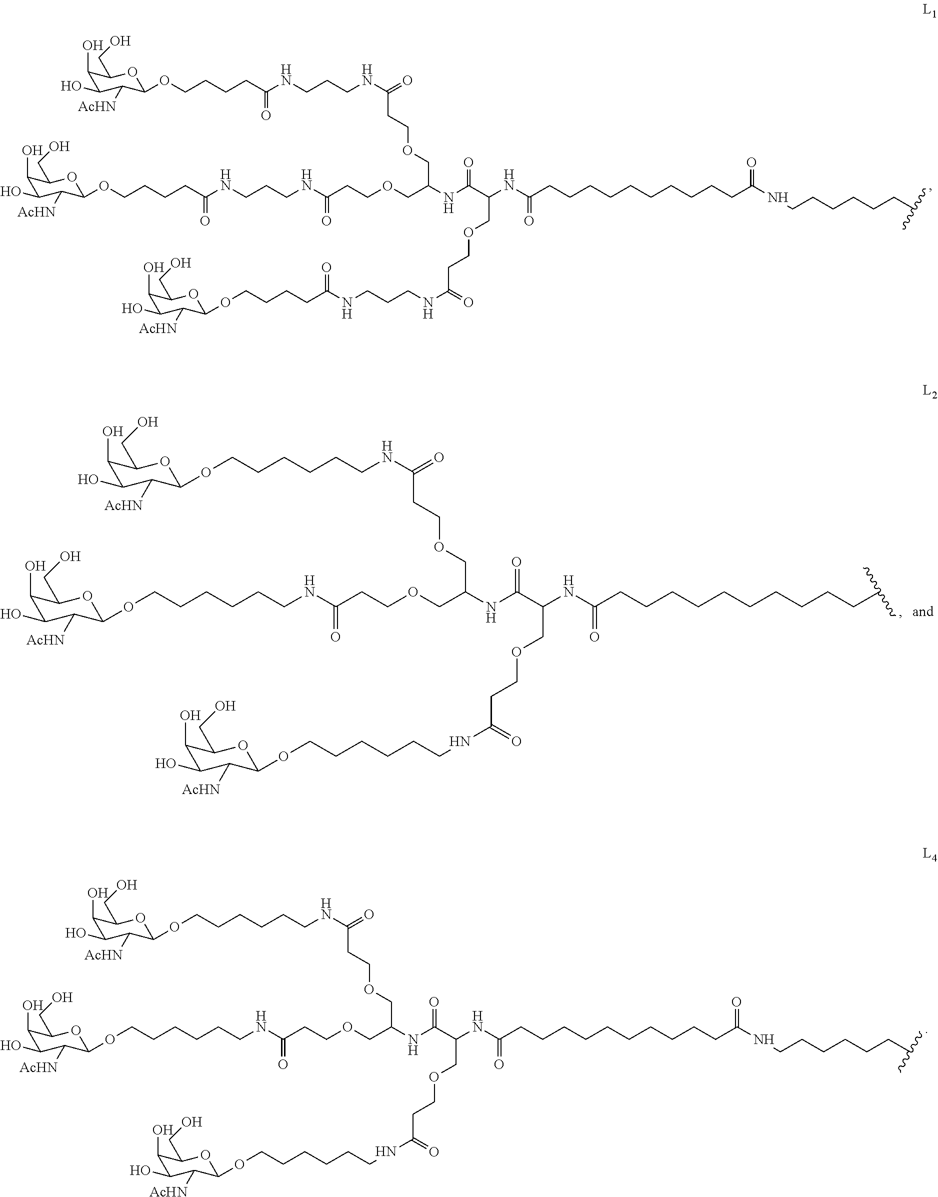

In more specific embodiment, this disclosure provides a conjugate of formula (III), or a pharmaceutical acceptable salt, tautomer or stereoisomer thereof, wherein, the said conjugate is selected from at least one of the following:

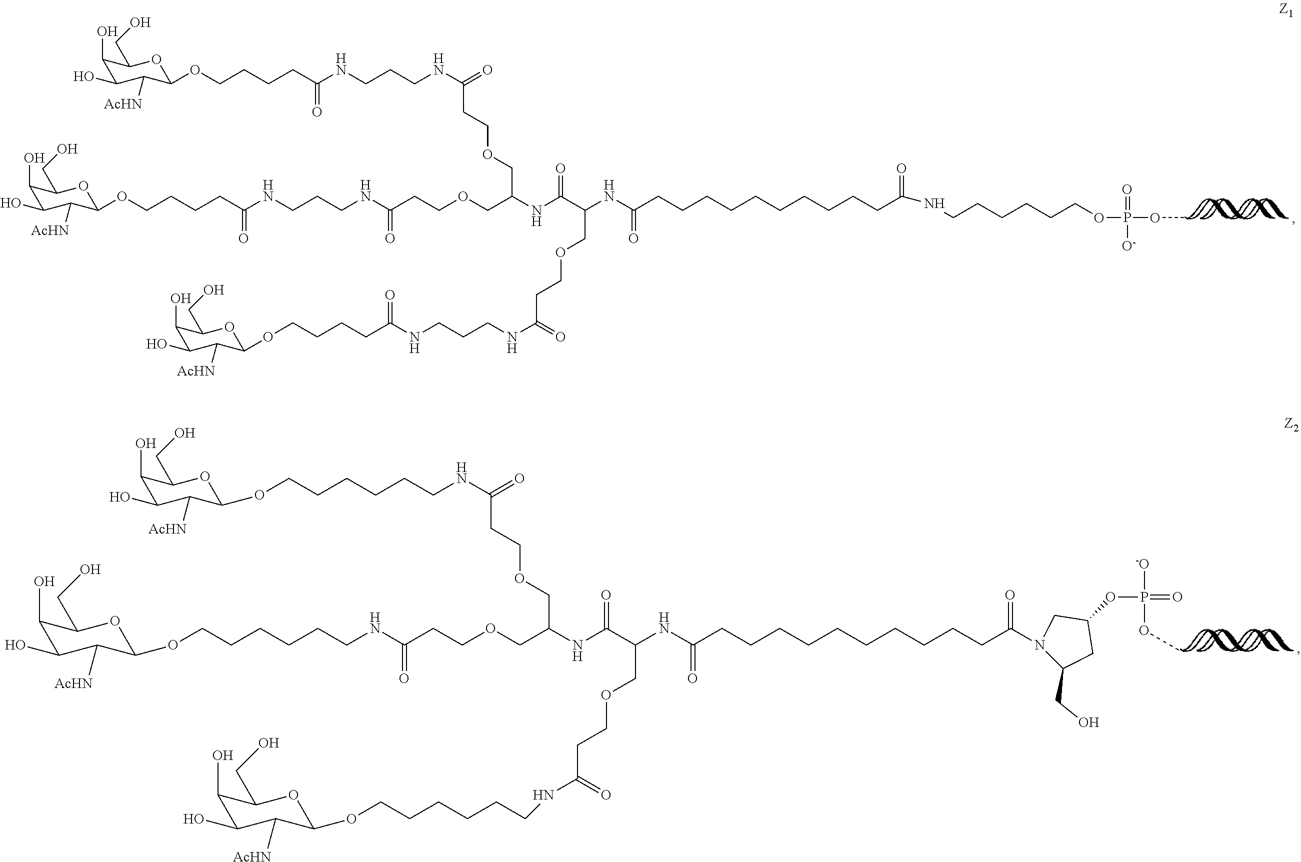

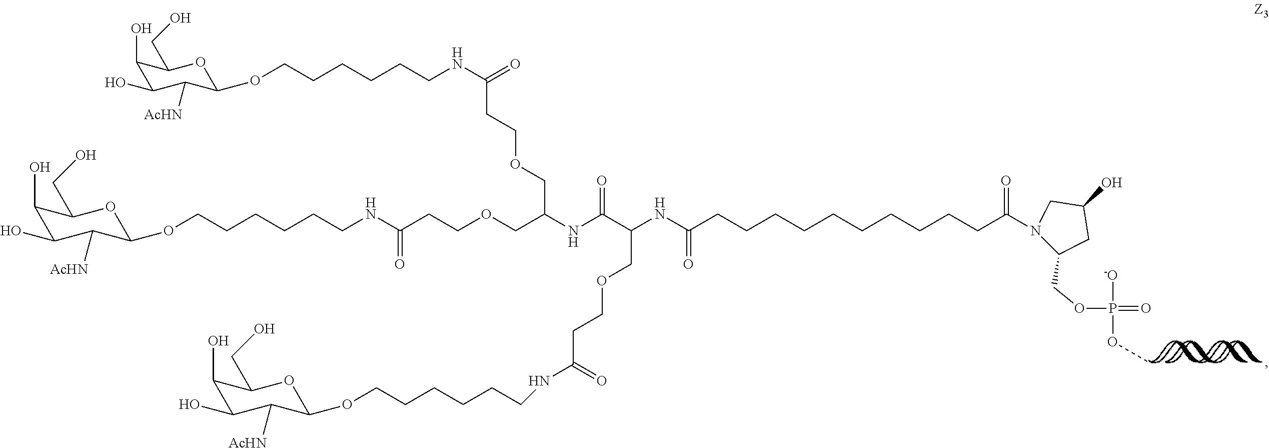

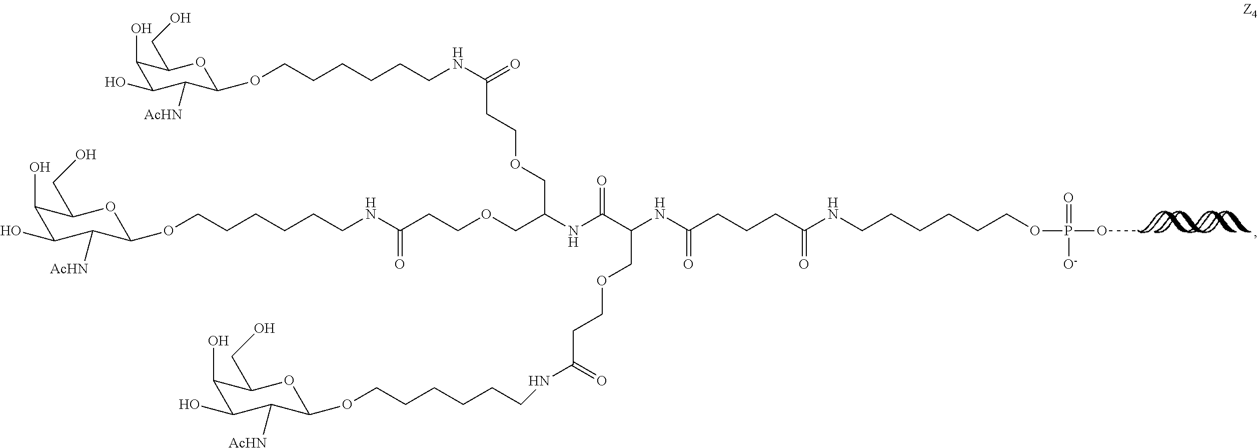

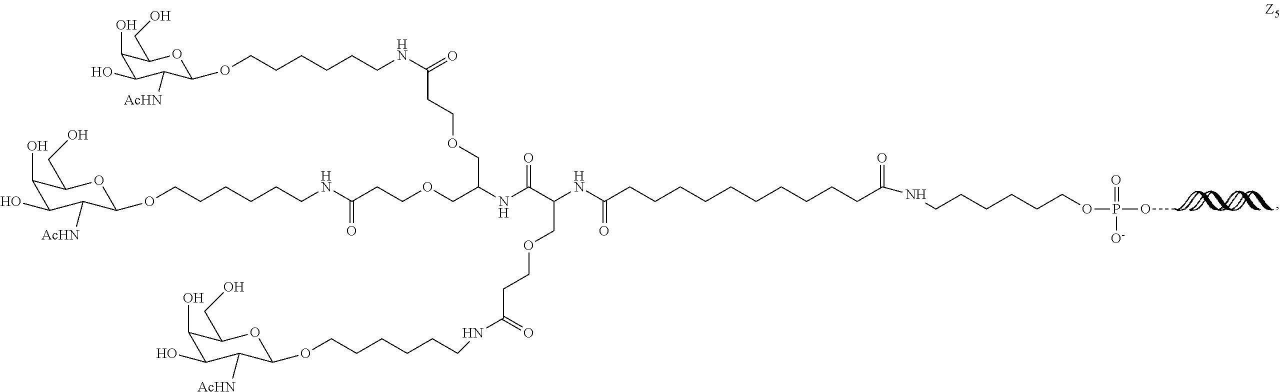

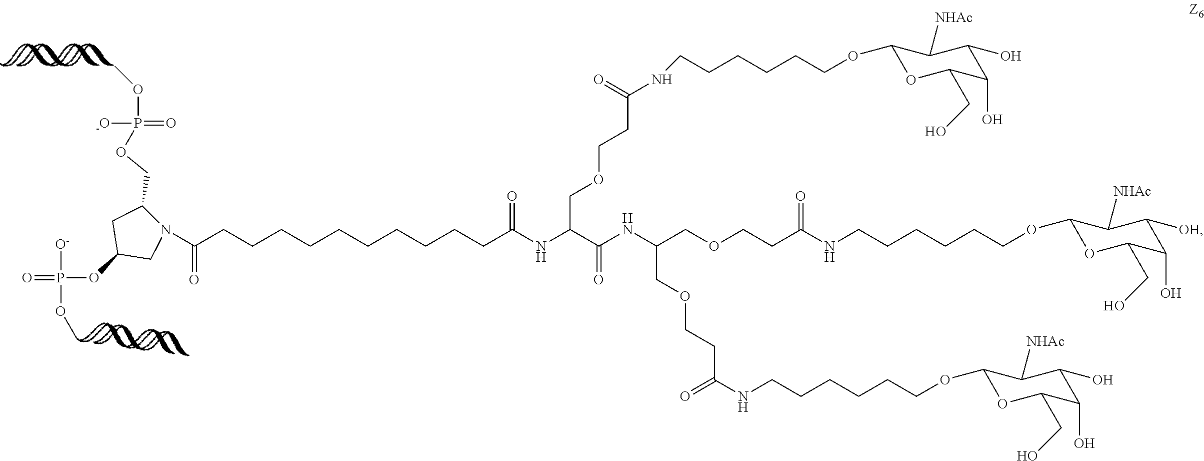

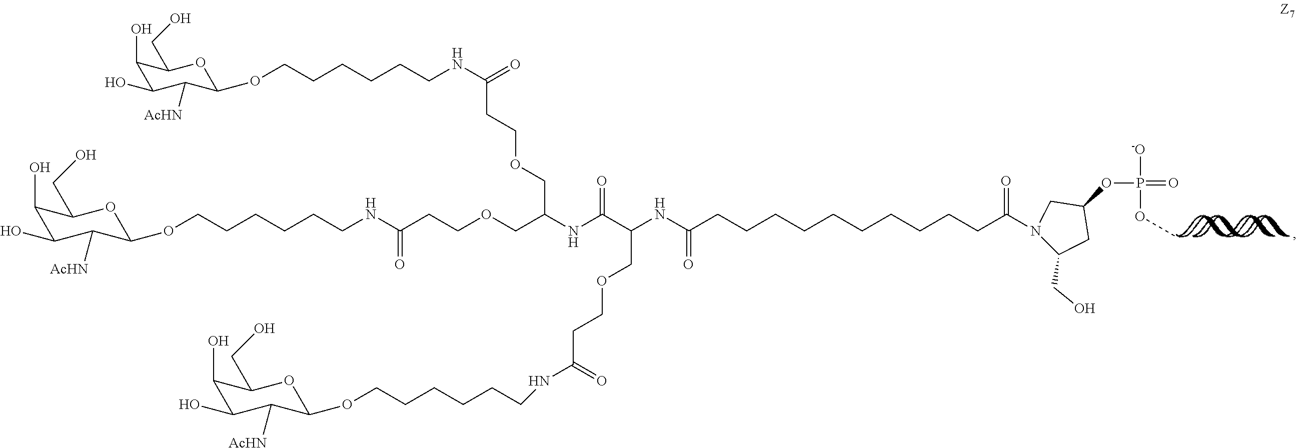

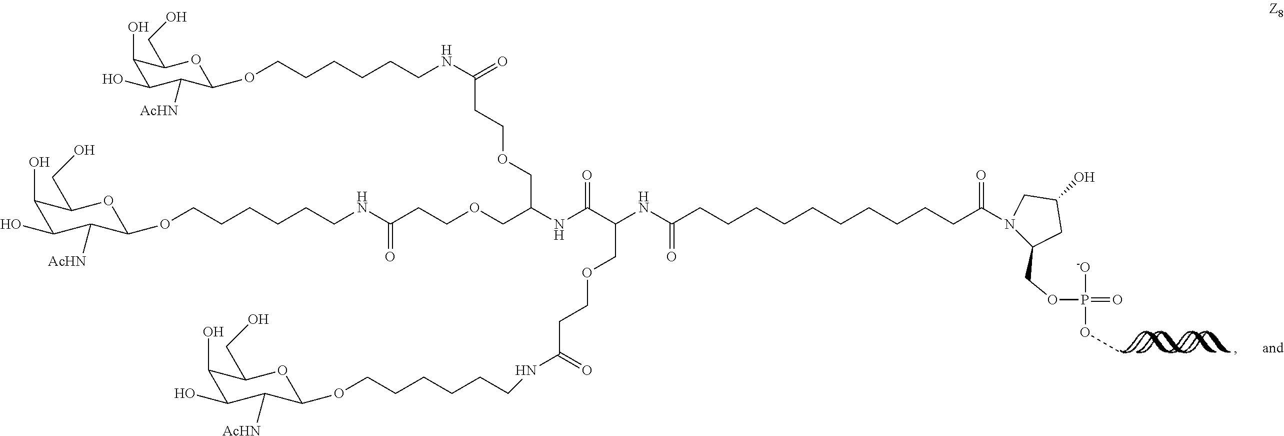

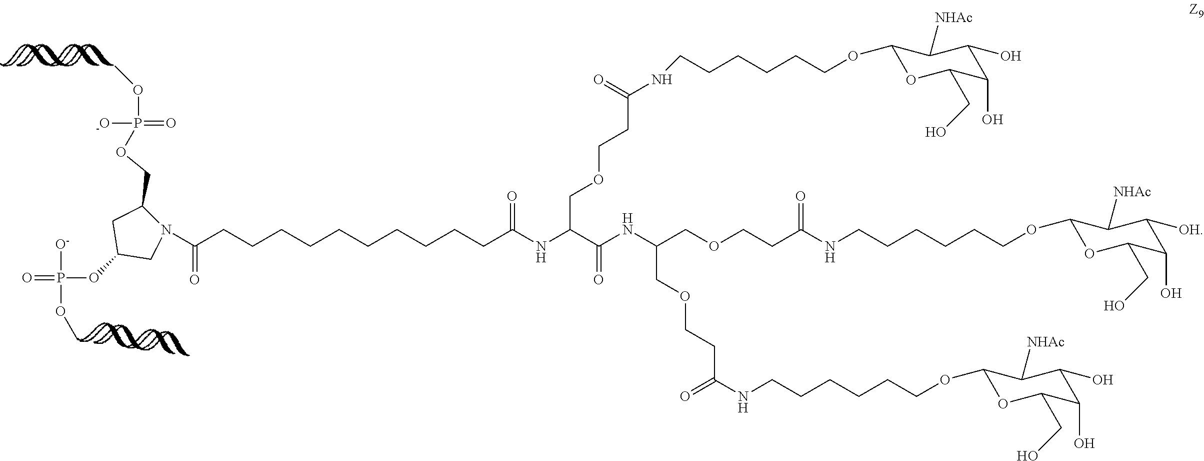

In more specific embodiment, this disclosure also provides a pharmaceutical composition, which includes conjugate of this disclosure, and optional selected pharmaceutical acceptable carrier, excipient, adjuvant or vehicle, and optionally other therapeutic agents.

In more specific embodiment, this disclosure also provides use of conjugate of this disclosure, or pharmaceutical composition of this disclosure in making pharmaceutical related to cure or prevent the disease related to expression or over expression of gene in hepatocyte.

In more specific embodiment, this disclosure also provides a method of curing or preventing the disease related to expression or over expression of gene in hepatocyte in subject, including administrating conjugate of this disclosure or pharmaceutical composition of this disclosure to subject.

In more specific embodiment, this disclosure also provides the conjugate of this disclosure or the pharmaceutical composition of this disclosure, which is used to cure or prevent the disease related to expression or over expression of gene in hepatocyte.

In more specific embodiment, wherein the said gene is selected from HBV gene group, HCV gene group, PCSK9, xanthine oxidase, URAT1, APOB, hepatic fibrosis related gene (AP3S2, AQP2, AZINI, DEGSI, STXBP5L, TLR4, TRPM5), non-alcoholic fatty liver disease related gene (PNPLA3, FDFTI), or primary biliary cirrhosis (HLA-DQB1, IL-12, IL-12RB2), or the combination thereof.

In more specific embodiment, wherein the said disease is selected from at least one of: hereditary angioedema, familial tyrosinemia type I or type II, Alagille syndrome, α-1-antitrypsindeficiency, bile acid synthesis and metabolism defect, biliary atresia, cystic fibrosis liver disease, idiopathic neonatal hepatitis, mitochondrial liver disease, progressive familial intrahepatic cholestasis, primary sclerosing cholangitis, transthyretin amyloidosis, hemophilia, homozygous familial hypercholesterolemia, hyperlipidemia, hepatitis B virus (HBV), hepatitis C virus (HCV), steatohepatitis, nonalcoholic steatohepatitis (NASH), non-alcoholic fatty liver disease (NAFLD), hyperglycemia or disease of abnormally increased hepatic glucose production similar to type I and II diabetes, hepatitis and hepatoporphyrin.

In more specific embodiment, this disclosure also provides a compound of formula (C):

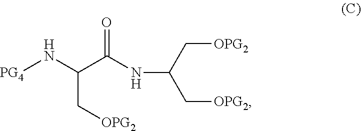

wherein, PG₂ and PG₄ are protecting groups;
preferably, PG₂ is hydroxyl protecting group, PG₄ is amino protecting group;
preferably, the said PG₂ is selected from at least one of trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS) and triisopropylsilyl (TIPS), preferably tert-butyldimethylsilyl (TBDMS);
preferably, the said PG₄ is selected from at least one of benzyloxycarbonyl (Cbz), tert-butoxycarbonyl (Boc), 9-fluorenylmethyloxycarbonyl (Fmoc), allyloxycarbonyl (Alloc), 2-(trimethylsilyl)ethoxycarbonyl (Teoc), methoxycarbonyl and ethoxycarbonyl, preferably tert-butoxycarbonyl (Boc).

In more specific embodiment, this disclosure also provides a making method of the formula (C) compound, which includes the following steps: compound A reacts with compound B to form compound C:

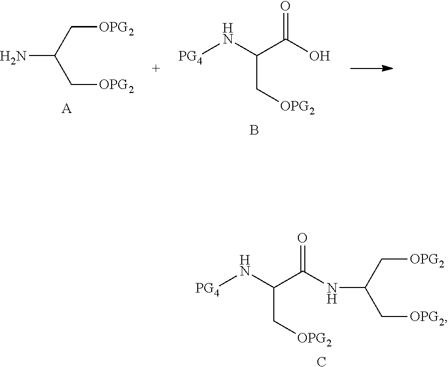

wherein, PG₂ and PG₄ are as defined in this disclosure;
preferably, the reaction condition of the said method includes condensation reagent and base;
preferably, the said condensation reagent is selected from at least one of carbodiimide condensation reagent, onium salt condensation reagent and organic phosphorus condensation reagent;
preferably, the said reaction condition also includes condensation activator;
preferably, the said reaction condition also includes solvent;
preferably, the said method is reacting in nitrogen atmosphere;
preferably, the said base is organic amine base, preferably at least one of DIEA(N, N-diisopropylethylamine), TEA(triethylamine), NMM(N-methylmorpholine), DBU(1,8-diazabicyclo[5.4.0]undec-7-ene), DMAP(4-dimethylaminopyridine), pyridine, 2,6-dimethylpyridine and 2,2,6,6-tetramethylguanidine, preferably DIEA.

Preferably, the said carbodiimide condensation reagent is selected from at least one of DCC (N,N′-dicyclohexylcarbodiimide), DIC (N,N′-diisopropylcarbodiimide) and EDCl (1-(3-dimethylaminopropyl)-3-ethylcarbodiimide hydrochloride), preferably EDCl.

Preferably, the said onium salt condensation reagent is selected from at least one of HATU (N,N,N′,N′-tetramethyl-O-(7-azabenzotriazol-1-yl)uronium hexafluorophosphate), HBTU (O-benzotriazol-1-yl-N,N,N′,N′-tetramethyluronium hexafluorophosphate), HCTU (O-(6-chloro-1-hydrocibenzotriazol-1-yl)-1,1,3,3-tetramethyluronium hexafluorophosphate), TBTU (2-(1H-benzotriazole-1-yl)-1,1,3,3-tetramethyluronium tetrafluoroborate), BOP (Castros reagent) and PyBOP (benzotriazole-1-yl-oxytripyrrolidinophosphonium hexafluorophosphate).

Preferably, the said organic phosphorus condensation reagent is selected from at least one of DPP-Cl (diphenylphosphinyl chloride), DPPA (diphenylazidophosphate) and BOP-Cl (bis(2-oxo-3-oxazolidinyl)phosphinic chloride).

Preferably, the said condensation reagent is selected from at least one of DMAP (4-dimethylaminopyridine), HOBt (1-hydroxybenzotriazole) and HOAt (1-hydroxy-7-azabenzotriazole), preferably HOBt.

Preferably, the said solvent is selected from at least one of DCM (dichloromethane), DCE (1,2-dichloroethane), DMF (N,N-dimethyl formamide), DMAC (N,N-dimethylacetamide), tetrahydrofuran and 2-methyltetrahydrofuran, preferably DCM.

Preferably, the molar ratio of compound A: compound B is (0.5-3):1, preferably (1-2):1, preferably (1-1.5):1, preferably 1.1:1.

Preferably, the molar ratio of condensation reagent: compound B is (1-3):1, preferably (1-2):1, preferably (1-1.5):1, preferably 1.2:1.

Preferably, the molar ratio of condensation activator: compound B is (1-3):1, preferably (1-2):1, preferably (1.2-1.8):1, preferably 1.5:1.

Preferably, the molar ratio of base: compound B is (0.5-5):1, preferably (1-4):1, preferably (1-3):1, preferably 2:1.

Preferably, the said method is carried out at about −20° C. to about 50° C., preferably about −10° C. to about 25° C., preferably −10° C. to about 0° C.

In more specific embodiment, this disclosure also provides a compound of formula (D):

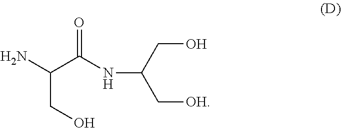 [SMILES: NC(CO)C(=O)NC(CO)CO]

In more specific embodiment, this disclosure also provides a making method of the formula (D) compound, which includes the following steps: the protecting group of the said compound C of this disclosure is removed to obtain compound D:

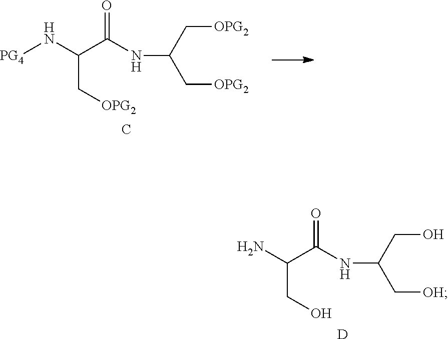

wherein, PG₂ and PG₄ are as defined in this disclosure;
preferably, the said method is reacted under acidic condition;
preferably, the reaction condition of the said method includes acid and solvent;
preferably, the said acid is HCl and/or H₂SO₄, preferably HCl;
preferably, the said solvent is organic solvent, preferably alcoholic solvent, preferably at least one of methanol, ethanol, propanol, butanol, isopropanol, cyclohexanol, 2-methyl-2-propanol and 2-ethyl-1-propanol, preferably methanol.

Preferably, the concentration of HCl in the reaction system is 0.1 mol/L to 3 mol/L, preferably 0.2 mol/L to 2 mol/L, preferably 0.5 mol/L to 1.5 mol/L, preferably 0.5 mol/L to 1.0 mol/L, preferably 0.75 mol/L.

Preferably, the said method is carried out in a nitrogen atmosphere.

Preferably, the said method is carried out at about −20° C. to about 50° C., preferably about −10° C. to about 25° C., preferably −10° C. to about 0° C.

The compounds or conjugates of the present disclosure may include one or more asymmetric centers, and thus may exist in a variety of stereoisomeric forms, for example, enantiomers and/or diastereomers. For example, the compounds of the present disclosure may be in the form of an individual enantiomer, diastereomer or geometric isomer (e.g., cis- and trans-isomers), or may be in the form of a mixture of stereoisomers, including racemic mixture and a mixture enriched in one or more stereoisomers. The isomers can be separated from the mixture by the methods known to those skilled in the art, including chiral high pressure liquid chromatography (HPLC) and the formation and crystallization of chiral salts; or preferred isomers can be prepared by asymmetric synthesis.

The compounds or the conjugates of the present disclosure may exist in tautomer forms. The tautomer is a functional group isomer resulting from the rapid shift of an atom between two positions in a molecule. The tautomer is a special functional group isomer, wherein a pair of tautomers can convert between each other, but usually exist in a relatively stable isomer as its main form.

### Pharmaceutical Compositions, Formulation and Kits

The present disclosure provides a pharmaceutical composition comprising a compound or a conjugate of the present disclosure (also referred to as the “active ingredient”) and a pharmaceutically acceptable carriers or excipient. In certain embodiments, the pharmaceutical composition comprises an effective amount of the active ingredient. In certain embodiments, the pharmaceutical composition comprises a therapeutically effective amount of the active ingredient. In certain embodiments, the pharmaceutical composition comprises a prophylactically effective amount of the active ingredient.

A pharmaceutically acceptable excipient for use in the present disclosure refers to a non-toxic carrier, adjuvant or vehicle which does not destroy the pharmacological activity of the compound or the conjugate formulated together. Pharmaceutically acceptable carriers, adjuvants, or vehicles that may be used in the compositions of the present disclosure include, but are not limited to, ion exchangers, alumina, aluminum stearate, lecithin, serum proteins (e.g., human serum albumin), buffer substances (such as phosphate), glycine, sorbic acid, potassium sorbate, a mixture of partial glycerides of saturated plant fatty acids, water, salt or electrolyte (such as protamine sulfate), disodium hydrogen phosphate, potassium hydrogen phosphate, sodium chloride, zinc salt, silica gel, magnesium trisilicate, polyvinyl pyrrolidone, cellulose-based materials, polyethylene glycol, sodium carboxymethyl cellulose, polyacrylate, wax, polyethylene-polyoxypropylene block polymers, polyethylene glycol and lanolin.

The present disclosure also includes kits (e.g., pharmaceutical packs). Kits provided may include a compound disclosed herein, other therapeutic agent(s), and a first and a second containers (e.g., vials, ampoules, bottles, syringes, and/or dispersible packages or other materials) containing the compound disclosed herein or other therapeutic agent(s). In some embodiments, kits provided can also optionally include a third container containing a pharmaceutically acceptable excipient for diluting or suspending the compound disclosed herein and/or other therapeutic agent(s). In some embodiments, the compound disclosed herein provided in the first container and the other therapeutic agent(s) provided in the second container is combined to form a unit dosage form.

The pharmaceutical composition provided by the present disclosure can be administered by a variety of routes including, but not limited to, oral administration, parenteral administration, inhalation administration, topical administration, rectal administration, nasal administration, oral administration, vaginal administration, administration by implant or other means of administration. For example, parenteral administration as used herein includes subcutaneous administration, intradermal administration, intravenous administration, intramuscular administration, intra-articular administration, intraarterial administration, intrasynovial administration, intrasternal administration, intrameningeal administration of cerebral spinal cord, intralesional administration, and intracranial injection or infusion techniques.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the disorder disclosed herein, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions provided herein can also be administered chronically (“chronic administration”). Chronic administration refers to administration of a compound or pharmaceutical composition thereof over an extended period of time, e.g., for example, over 3 months, 6 months, 1 year, 2 years, 3 years, 5 years, etc, or may be continued indefinitely, for example, for the rest of the subject's life. In certain embodiments, the chronic administration is intended to provide a constant level of the compound in the blood, e.g., within the therapeutic window over the extended period of time.

The pharmaceutical compositions of the present disclosure may be further delivered using a variety of dosing methods. For example, in certain embodiments, the pharmaceutical composition may be given as a bolus, e.g., in order to raise the concentration of the compound in the blood to an effective level soon. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

Transdermal doses are generally selected to provide similar or lower blood levels than are achieved using injection doses, generally in an amount ranging from about 0.01 to about 20% by weight, preferably from about 0.1 to about 20% by weight, preferably from about 0.1 to about 10% by weight, and still preferably from about 0.5 to about 15% by weight.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

Injectable compositions are typically based upon injectable sterile saline or phosphate-buffered saline or other injectable excipients known in the art. As before, the active compound in such compositions is typically a minor component, often being from about 0.05 to 10% by weight with the remainder being the injectable excipient and the like.

The above-described components for injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present disclosure can also be administered in sustained release forms or from sustained release drug delivery systems. A description of representative sustained release materials can be found in Remington's Pharmaceutical Sciences.

The present disclosure also relates to the pharmaceutically acceptable formulations of a compound of the present disclosure. In one embodiment, the formulation comprises water.

### Pharmaceutical Combination

The compounds or the conjugates described herein can be used in combination with one or more other active ingredients in pharmaceutical compositions or methods to treat the diseases and disorders described herein. Other additional active ingredients include other therapeutic agents or drugs that alleviate the adverse effects of the therapeutic agent on the expected disease target. The combination can be used to increase efficacy, improve symptoms of other diseases, reduce one or more negative effects, or reduce the required dosage of the compound of the present disclosure. Additional active ingredients can be formulated into a pharmaceutical composition separated from that of the compounds of the present disclosure or can be included in a single pharmaceutical composition with the compounds of the present disclosure. The additional active ingredient can be administered simultaneously with, before or after the administration of the compound of the present disclosure.

Combination agents include those additional active ingredients that known or observed to be effective in the treatment of the diseases and conditions described herein, including those effectively against another target related to the disease. For example, the compositions and formulations of the present disclosure, and treatment methods may further include other drugs or medicines, such as other active agents that can be used to treat or alleviate the target disease or related symptoms or conditions. The pharmaceutical composition of the present disclosure may additionally include one or more of the active agents, and the method of treatment may additionally include administering an effective amount of one or more of the active agents.

The beneficial effects of the present disclosure are:
(1) this disclosure uses serine as the starting material to produce a new molecule peptide skeleton with a carbon atoms in serine as central branch points, via the condensation reaction with serinol, the new molecule skeleton meets the following structural characteristics: (i) a central branch structure; (ii) the spacer is based on several peptide binding groups which combines flexibility and amphiphilicity; (iii) the spacer's distance between the bound central branch structure and GalNAc is 20-30 Å; (iv) the length of spacer can be easily changed.
(2) the new three cluster galactose type compound in this disclosure has better hydrophilicity and low toxicity; the binding affinity of the formed conjugate binds to ASGPR receptor is relatively high, Kd value is less than 10 and may low to about 2; compared with the negative control siRNA does not bind to GalNAc, the distribution of the conjugate of this disclosure in the mouse liver is obviously increased, which can achieve hepatic targeting delivery in vivo, obviously knock down the expression of target gene mRNA.
(3) the synthesis method of the trivalent GalNAc cluster derivatives based on new skeleton of this disclosure is simple, a mature phosphoramidite solid-phase synthesis coupling strategy is used to covalently couple with ASO, the clinical efficacy of ASO medicines can be improved without increasing the complexity or cost of the existing oligonucleotide medicines production scheme, and the existing, cheap and readily available raw materials can be used for effective assembly synthesis and large-scale production.

### BRIEF DESCRIPTION OF THE DRAWINGS

FIG. 1 is binding curve of G1-12 of this disclosure and ASGPR receptor.

FIG. 2 is binding curve of G4-12 of this disclosure and ASGPR receptor.

FIG. 3 is binding curve of G5-12 of this disclosure and ASGPR receptor.

FIG. 4 is testing result of G1-12 of this disclosure applied in in vivo hepatic targeting testing in mouse.

### EXAMPLES

The following is a clear and complete description of the technical solution of the disclosure in combination with the drawings. Obviously, the described embodiments are only part of rather than all of the embodiments of the disclosure. All other embodiments obtained by persons of ordinary skill in the art based on the embodiments of the disclosure without creative efforts shall fall within the protection scope of the disclosure.

### Example 1: Synthesis of G1

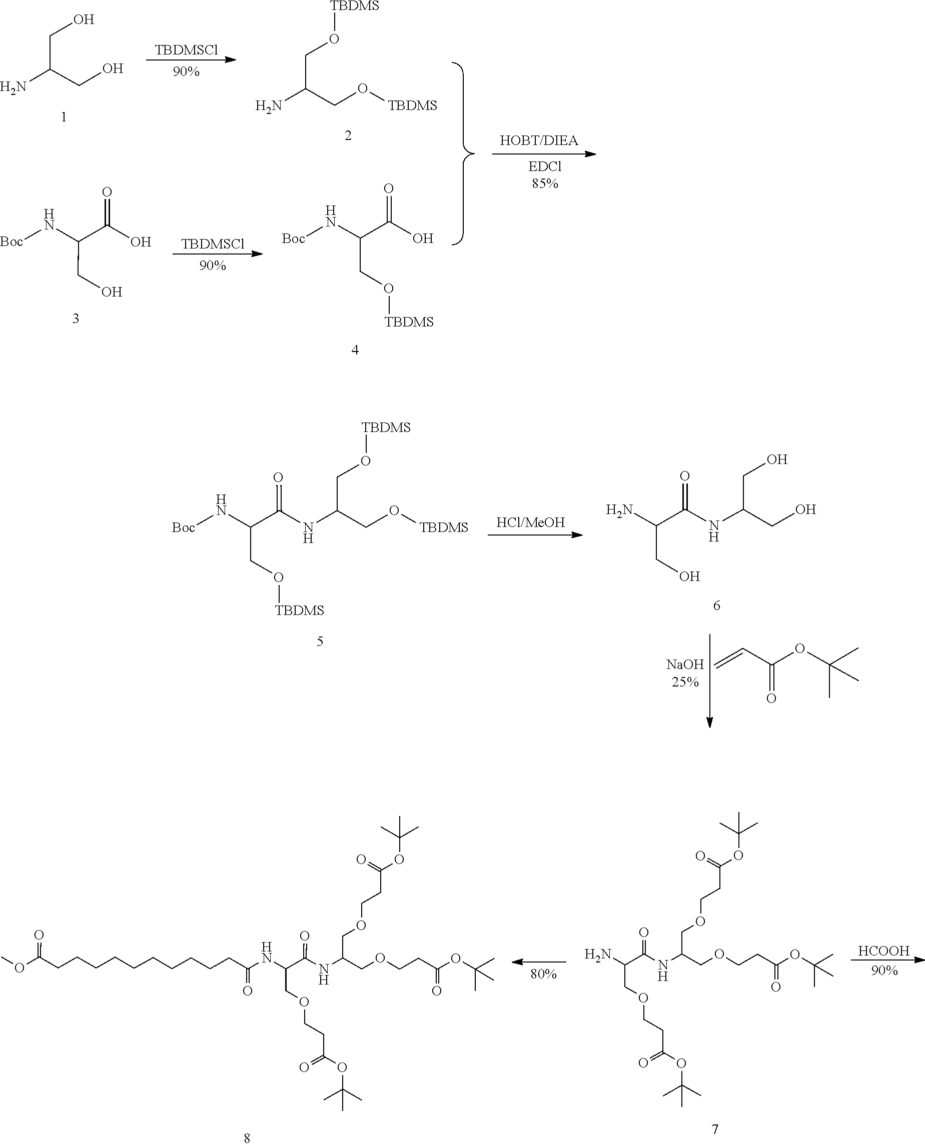

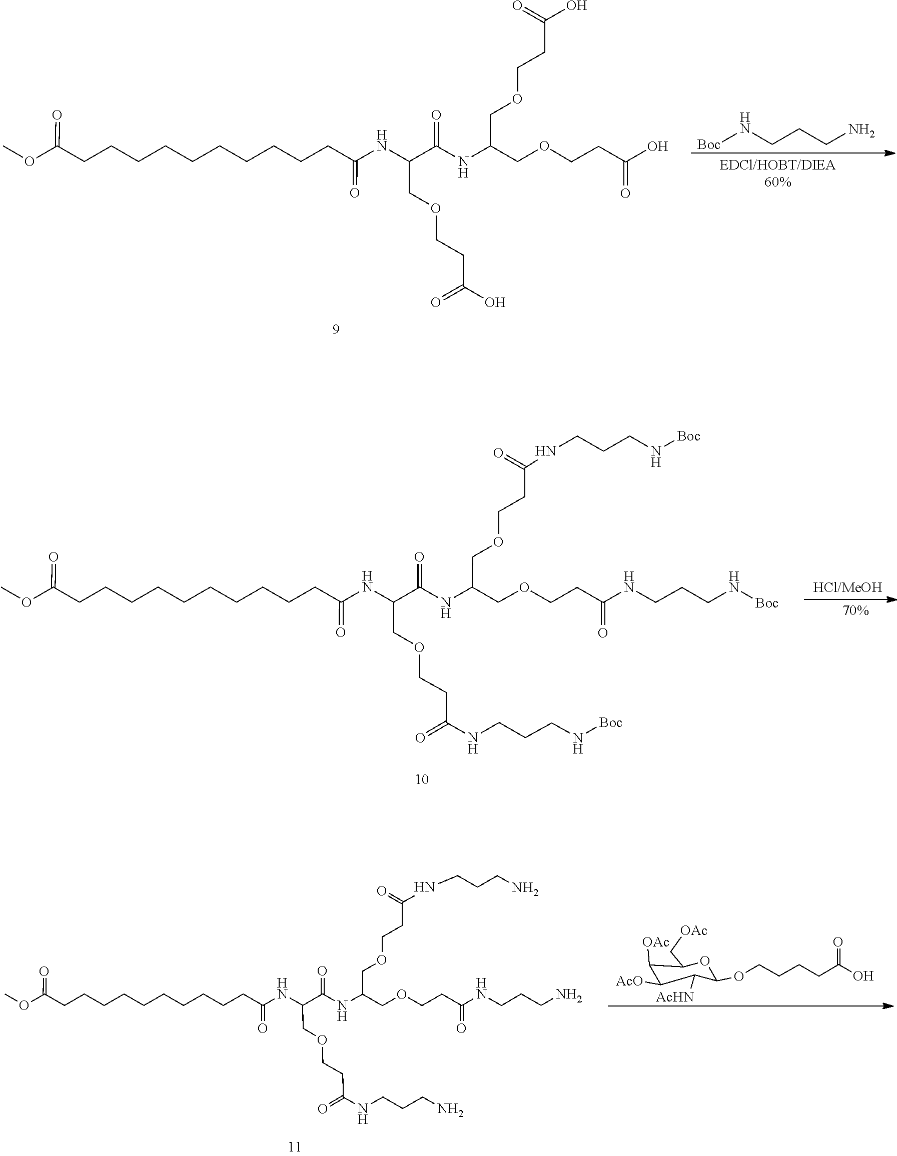 [SMILES: COC(=O)CCCCCCCCCCC(=O)NC(COCCC(=O)O)C(=O)NC(COCCC(=O)O)COCCC(=O)O ; CC(C)(C)OC(=O)NCCCN ; COC(=O)CCCCCCCCCCC(=O)NC(COCCC(=O)NCCCNC(=O)OC(C)(C)C)C(=O)NC(COCCC(=O)NCCCNC(=O)OC(C)(C)C)COCCC(=O)NCCCNC(=O)OC(C)(C)C ; COC(=O)CCCCCCCCCCC(=O)NC(COCCC(=O)NCCCN)C(=O)NC(COCCC(=O)NCCCN)COCCC(=O)NCCCN ; CC(=O)NC1C(OCCCCC(=O)O)OC(COC(C)=O)C(OC(C)=O)C1OC(C)=O]

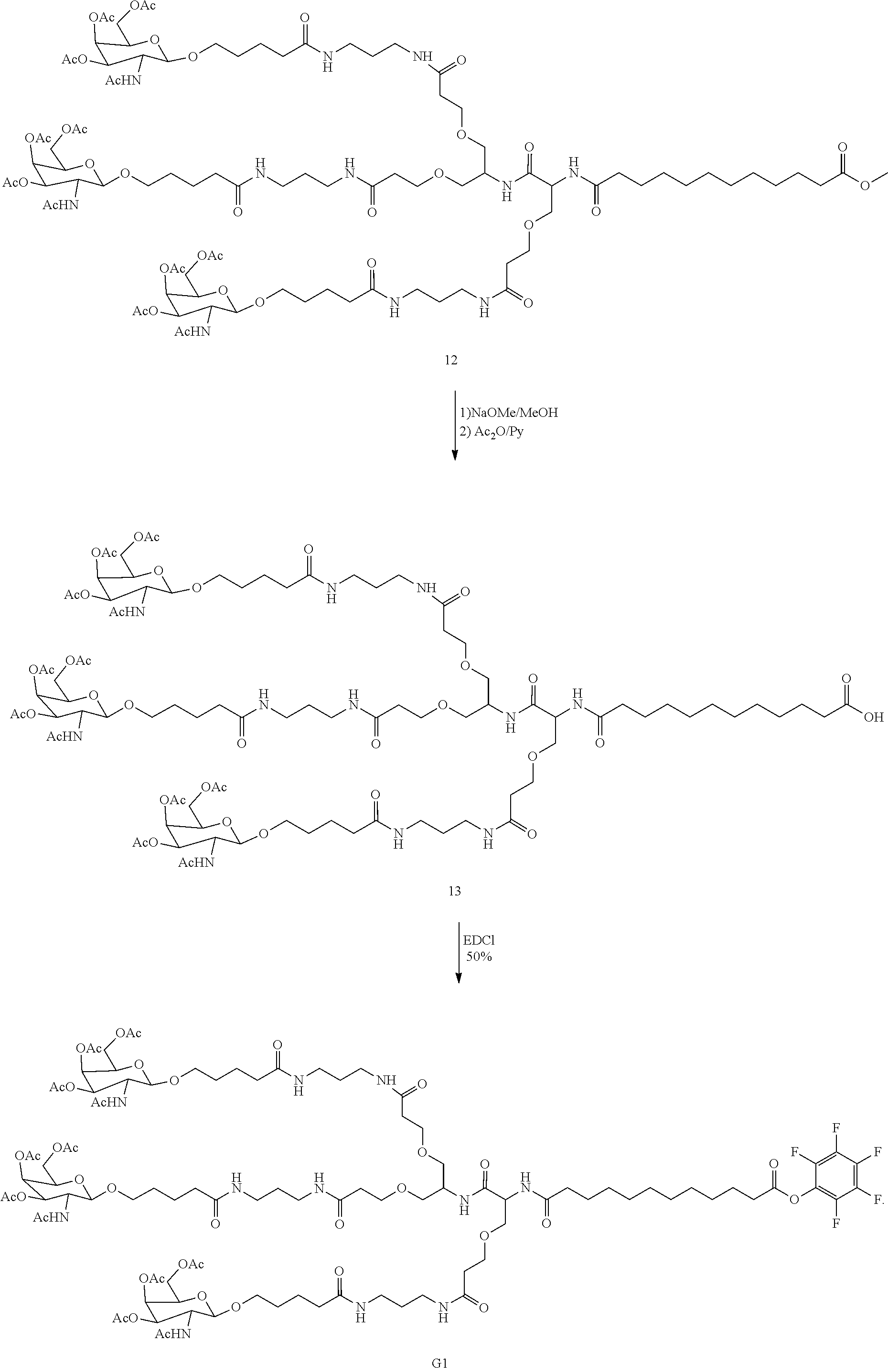

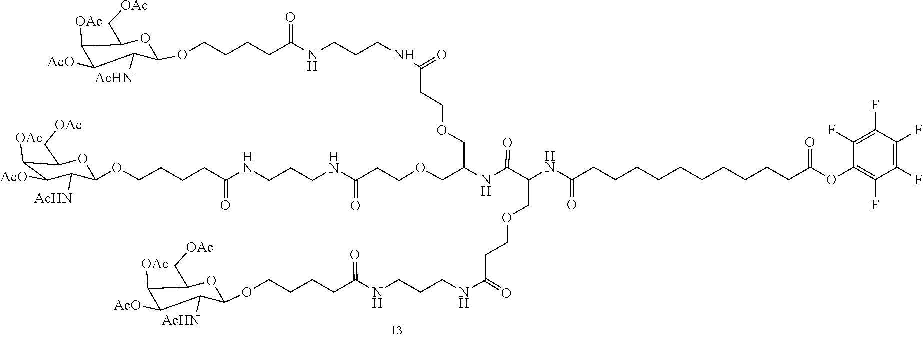 [SMILES: CC(=O)NC1C(OCCCCC(=O)NCCCNC(=O)CCOCC(COCCC(=O)NCCCNC(=O)CCCCOC2OC(COC(C)=O)C(OC(C)=O)C(OC(C)=O)C2NC(C)=O)NC(=O)C(COCCC(=O)NCCCNC(=O)CCCCOC2OC(COC(C)=O)C(OC(C)=O)C(OC(C)=O)C2NC(C)=O)NC(=O)CCCCCCCCCCC(=O)Oc2c(F)c(F)c(F)c(F)c2F)OC(COC(C)=O)C(OC(C)=O)C1OC(C)=O]

Compound 1 (100 g) was dissolved in dry DCM (500 mL), DMAP (1 g), Et₃N (5 eq) were added under nitrogen protection in ice bath, TBDMSCl (2.5 eq) was added slowly, after reaction completed, NaHCO₃ solution was used to wash, compound 2 was obtained by chromatography.

Compound 3 (100 g) was dissolved in anhydrous DMF (500 mL), imidazole (3.2 eq) was added under nitrogen protection in ice bath, TBDMSCl (1.4 eq) was added slowly, after reaction completed, compound 4 was obtained by washing with saturated NaHCO₃ solution and saline, concentration and chromatography, successively.

Compound 4 (50 g) was dissolved in anhydrous DCM (30 mL), HOBt (1.5 eq) was added under nitrogen protection in ice bath, EDCl (1.2 eq) was added slowly, after 20 minutes, compound 2 (1.1 eq), DIEA(2 eq) were added under nitrogen protection in ice bath, after reaction completed, compound 5 was obtained by washing with saturated NaHCO₃ solution, 10% citric acid aqueous solution and saturated saline, concentration and chromatography, successively.

Compound 5 (30 g) was dissolved in anhydrous MeOH (60 mL), HCl/MeOH solution (20 mL, 3 mol/L) was added under nitrogen protection in ice bath, the reaction was reacted at room temperature, after reaction completed, compound 6 was obtained by washing with ethyl acetate and concentration.

Compound 6 (20 g) was dissolved in anhydrous DMSO (100 mL), NaOH (1.3 eq) was added under nitrogen protection in ice bath, tert-butyl acrylate (6 eq) was added slowly, after reaction completed, compound 7 was obtained by washing with EA and saturated saline, concentration and chromatography, successively.

Compound 7 (15 g) was dissolved in anhydrous DCM (300 mL), methyl laurate (1.1 eq), HOBt (1.5 eq) were added under nitrogen protection in ice bath, EDCl (1.2 eq) was added slowly, after 20 minutes, DIEA (2 eq) was added, compound 8 was obtained by washing with saturated NaHCO₃ solution, 10% citric acid aqueous solution and saturated saline, concentration and chromatography, successively.

Compound 8 (15 g) was dissolved in HCOOH (30 mL), after reaction completed, compound 9 was obtained by concentration and chromatography.

Compound 9 (10 g) was dissolved in anhydrous DMF (60 mL), HOBt (1.25 eq), EDCl (1.2 eq) and N-Boc-1,3-propanediamine (1.2 eq) were added, DIEA (2 eq) was added under nitrogen protection in ice bath, after reaction completed, compound 10 was obtained by washing with saturated NaHCO₃ solution and 0.5 N HCl solution, and chromatography, successively.

Compound 10 (9.3 g) was dissolved in anhydrous DCM (60 mL), HCl/MeOH solution (20 mL, 2 mol/L) was added under nitrogen protection in ice bath, after reaction completed, compound 11 was obtained.

N-acetylgalactosamine (15 g) was dissolved in anhydrous DMF (100 mL), HOBt (1.5 eq) was added under nitrogen protection in ice bath, EDCl (1.2 eq) was added slowly, after 20 minutes, DIEA (2 eq) and compound 11 (9.3 g) were added, after reaction completed, compound 12 was obtained by washing with saturated NaHCO₃ solution, 0.5 N HCl solution and saturated saline, concentration and chromatography, successively.

Compound 12 (4 g) was dissolved in anhydrous MeOH (20 mL), NaOMe (30% in MeOH, 3 mL) was added under nitrogen protection in ice bath, after reaction completed, crude product was obtained by concentration, the crude product was added in MeOH/H₂O and reacted at room temperature, after reaction completed, Et₃N hydrochloride was used to adjust pH to neutralization, crude product was obtained by concentration, then the crude product was added in Py (20 mL) and Ac₂O (5 mL) and reacted at room temperature, after reaction completed, compound 13 was obtained by chromatography.

Compound 13 (1 g) was dissolved in anhydrous DCM (10 mL), EDCl (1.5 eq) and pentafluorophenol (1.5 eq) were added, after reaction completed, compound G1 (0.5 g) was obtained by washing with saturated NaHCO₃ solution, concentration and chromatography, the productivity was 50%.

¹H NMR (500 MHz, CDCl₃) δ: 7.61-7.30 (m, 3H), 5.31-5.19 (m, 3H), 5.03 (d, J=11.1 Hz, 3H), 4.49 (d, J=8.3 Hz, 3H), 4.15-3.96 (m, 9H), 3.86 (m, 6H), 3.71-3.55 (m, 9H), 3.50-3.39 (m, 12H), 3.16 (m, 6H), 2.59 (t, J=7.6 Hz, 2H), 2.35 (dt, J=4.9 Hz, 6H), 2.26-2.03 (m, 20H), 2.03-1.81 (m, 30H), 1.55 (m, 8H), 1.19 (m, 20H), ESI-Tof-MS m/z: 2264.10[M+Cl]⁻; C₉₉H₁₅₀F₅N₁₁O₄₀.

### Example 2: Synthesis of G2

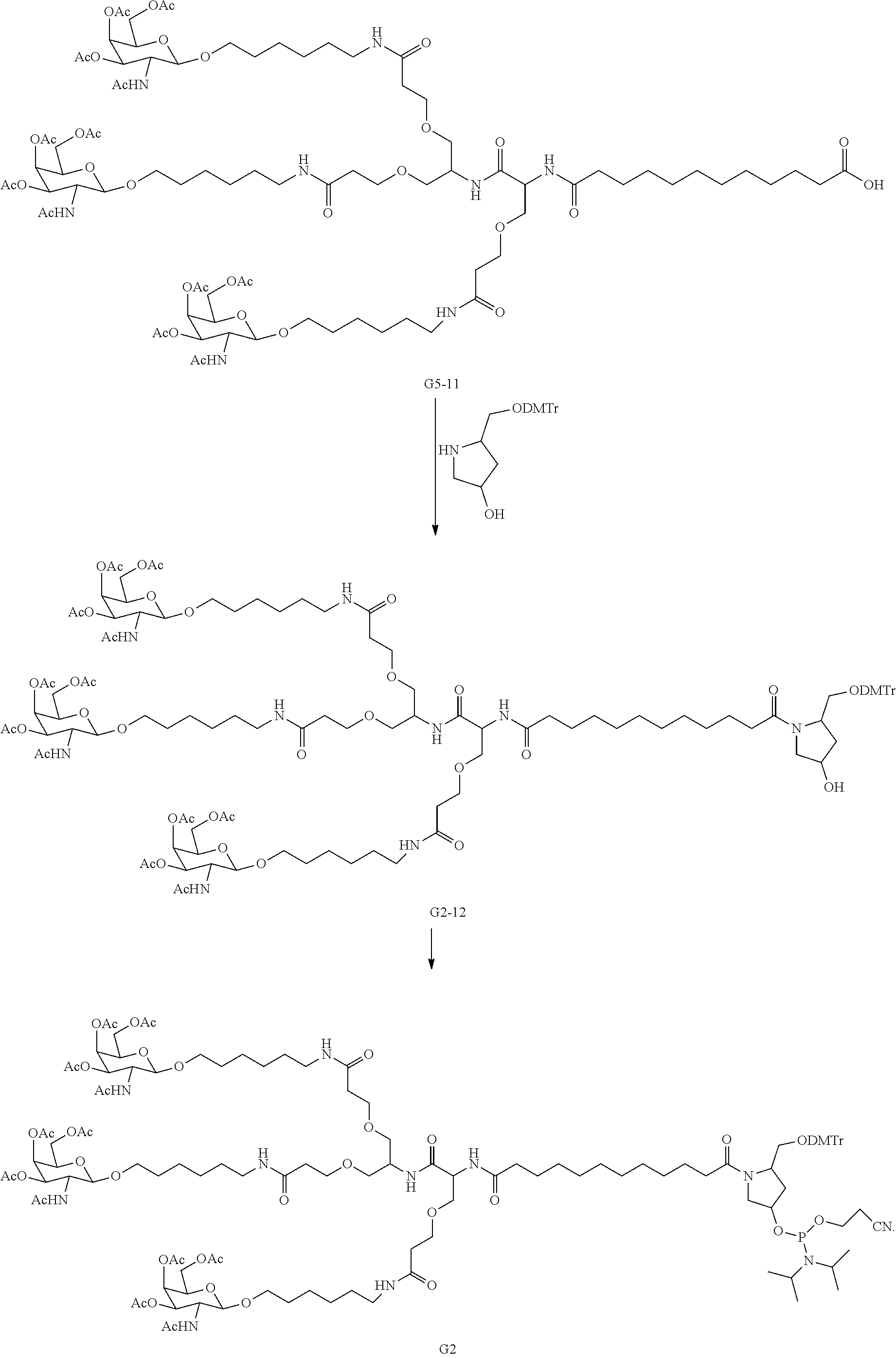

Compound G5-11 (2 g) was dissolved in DCM (10 mL), HBTU (1.2 eq), HOBT (3 eq), DIPEA (3 eq) and proline (1.05 eq) were added, after reaction completed at room temperature, crude compound was obtained by washing with saturated NaHCO₃ solution and concentration, compound G2-12 was obtained by chromatography.

Compound G2-12 (1 g) was dissolved in anhydrous DCM (10 mL), DIPEA (3 eq) was added, phosphoramidite (1.5 eq) was added under nitrogen protection in ice bath, after reaction completed, compound G2 was obtained by washing with saturated NaHCO₃ solution, concentration and chromatography, the productivity was 41%.

¹H NMR (500 MHz, CDCl₃): δ 7.29 (t, J=7.9 Hz, 2H), 7.24-7.06 (m, 10H), 6.91 (s, 1H), 6.78-6.69 (m, 6H), 6.62-6.41 (m, 3H), 5.28 (s, 3H), 5.19 (d, J=9.2 Hz, 3H), 4.61 (s, 3H), 4.45 (m, 2H), 4.07 (m, 6H), 3.93 (m, 3H), 3.89-3.76 (m, 6H), 3.69 (m, 12H), 3.57-3.28 (m, 1 OH), 3.13 (m, 6H), 3.04 (m, 2H), 2.52 (dd, J=12.0, 5.7 Hz, 2H), 2.37 (m, 6H), 2.19 (m, 4H), 1.99 (m, 36H), 1.72 (m, 16H), 1.60-1.36 (m, 16H), 1.34-0.93 (m, 22H); ³¹PNMR (202 MHz, CDCl₃) δ: 147.37, (s). ESI-Tof-MS m/z: 2491.25[M−H]⁻; C₁₂₂H₁₈₆N₁₁O₄₁P.

### Example 3: Synthesis of G3

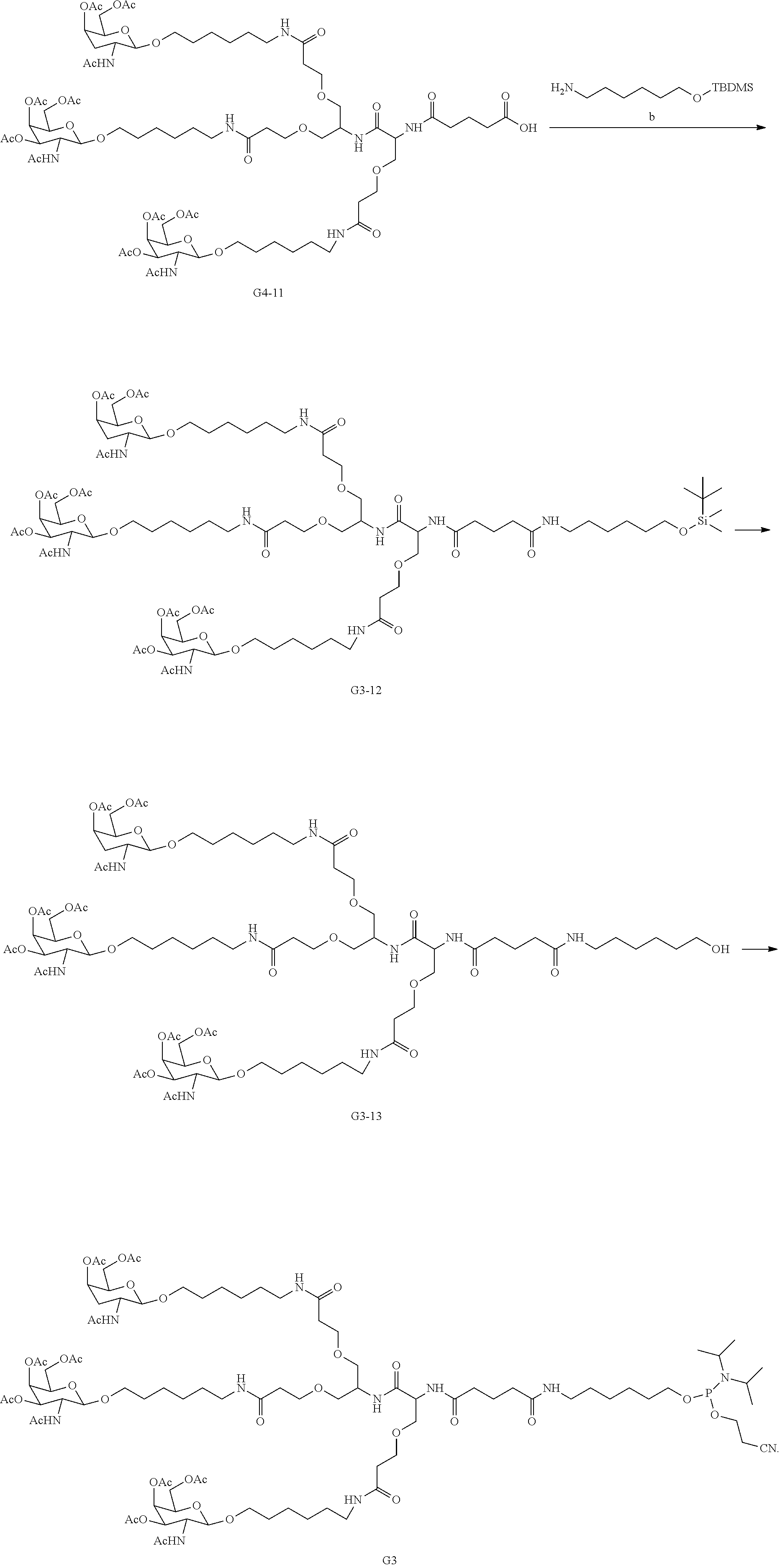

Compound G4-11 (2 g) was dissolved in DCM (10 mL), EDCl (1.5 eq), HOBT (2 eq), DIPEA (3 eq) and compound b (1.2 eq) were added, after reaction completed at room temperature, compound G3-12 (1.5 g) was obtained by washing with saturated NaHCO₃ solution, concentration and chromatography.

Compound G3-12 (2 g) was dissolved in THE (20 mL), triethylamine trihydrofluoride (1.5 eq) was added, after reaction completed at room temperature, DCM was added when concentration, compound G3-13 (1.3 g) was obtained by washing with saturated NaHCO₃ solution, concentration and chromatography.

Compound G3-13 (1.2 g) was dissolved in anhydrous DCM (10 mL), DIPEA (3 eq) was added, phosphoramidite monomer (1.5 eq) was added under nitrogen protection in ice bath, after reaction completed, compound G3 (0.6 g) was obtained by washing with saturated NaHCO₃ solution, concentration and chromatography, the productivity was 46%.

¹H NMR (500 MHz, CDCl₃): δ 7.16 (s, 1H), 7.04 (t, J=14.7 Hz, 2H), 6.83 (d, J=5.5 Hz, 2H), 6.69 (d, J=5.7 Hz, 1H), 6.56 (d, J=8.8 Hz, 2H), 6.42 (s, 1H), 5.35 (d, J=2.5 Hz, 3H), 5.30-5.23 (m, 3H), 4.68 (d, J=8.3 Hz, 3H), 4.21-4.06 (m, 8H), 3.99 (dd, J=19.4, 8.8 Hz, 3H), 3.94-3.83 (m, 6H), 3.63 (m, 6H), 3.46 (dd, J=15.8, 6.6 Hz, 4H), 3.34-3.16 (m, 16H), 2.68 (t, J=7.3 Hz, 2H), 2.42 (t, J=5.2 Hz, 6H), 2.33-2.22 (m, 4H), 2.21-1.88 (m, 38H), 1.79 (m, 14H), 1.63-1.43 (m, 18H), 1.30 (d, J=43.5 Hz, 12H); ³¹P NMR (202 MHz, CDCl₃) δ: 147.37, (s). ESI-Tof-MS m/z: 2033.40[M−H]⁻; C₉₃H₁₅₆N₁₁O₃₆P.

### Example 4: Synthesis of G4

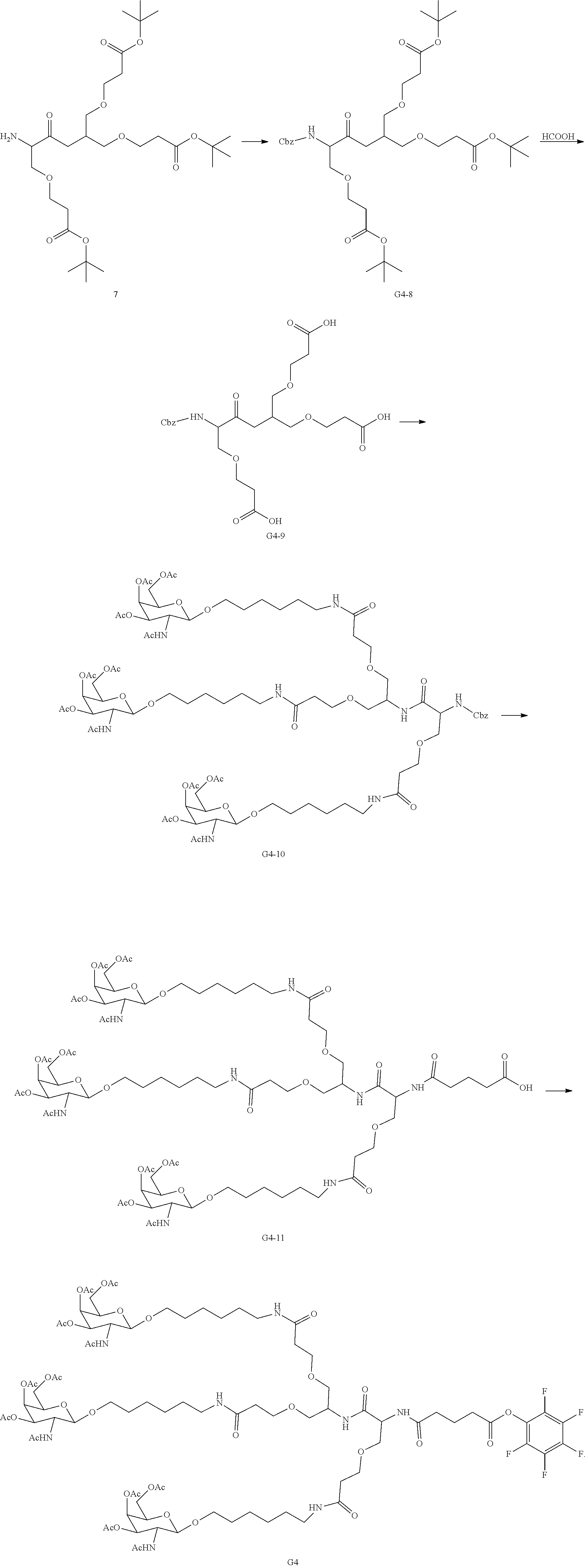

Compound 7 (25 g) was dissolved in anhydrous DCM (50 mL), Na₂CO₃ (200 mL, 25% aqueous solution) was added under nitrogen protection in ice bath, CbzCl (2 eq) was added slowly, after reaction completed, G4-8 (30 g) was obtained by washing with saturated saline, concentration and chromatography.

Compound G4-8 (8 g) was dissolved in HCOOH (30 mL), after reaction completed, G4-9 (5 g) was obtained by concentration and chromatography.

Compound G4-9 (8 g) was dissolved in anhydrous DCM (50 mL), HOBt (3.3 eq) was added under nitrogen protection in ice bath, EDCl (3.3 eq) was added slowly, after 20 minutes, N-acetylgalactosamine (3.3 eq) and DIEA (6 eq) were added, after reaction completed, compound G4-10 (6.4 g) was obtained by washing with saturated NaHCO₃ solution, 0.5 mol/L HCl solution and saturated saline, concentration and chromatography, successively.

Compound G4-10 (3 g) was dissolved in anhydrous THE (50 mL), succinic anhydride (1.5 eq) was added, 10% Pd/C (1 g) was added slowly, after reaction completed under hydrogen atmosphere, G4-11 (2.5 g) was obtained by filtration, concentration and chromatography.

Compound G4-11 (1 g) was dissolved in DCM (10 mL), EDCl (1.5 eq) and pentafluorophenol (1.5 eq) were added, after reaction completed, compound G4 (0.5 g) was obtained by washing with saturated NaHCO₃ solution, concentration and chromatography, the productivity was 40%.

1H NMR (500 MHz, CDCl₃): δ 7.29-7.20 (m, 2H), 7.11 (m, 1H), 6.91-6.68 (m, 3H), 6.56-6.39 (m, 2H), 5.34 (d, J=2.5 Hz, 3H), 5.25 (d, J=11.1 Hz, 3H), 4.66 (d, J=8.3 Hz, 3H), 4.71-4.50 (m, 2H), 4.13 (tt, J=18.0, 8.9 Hz, 6H), 3.98 (dd, J=18.4, 9.2 Hz, 3H), 3.88 (ddd, J=15.5, 11.8, 6.3 Hz, 6H), 3.79-3.63 (m, 6H), 3.56-3.35 (m, 9H), 3.21 (td, J=12.5, 6.2 Hz, 6H), 2.76 (t, J=7.3 Hz, 2H), 2.43 (dd, J=15.3, 7.7 Hz, 8H), 2.19-1.86 (m, 38H), 1.62-1.42 (m, 12H), 1.28 (m, 12H). ESI-MS m/z: 1957.30[M−H]⁻; C₈₆H₁₂₇F₅N₈O₃₇.

### Example 5: Synthesis of G5

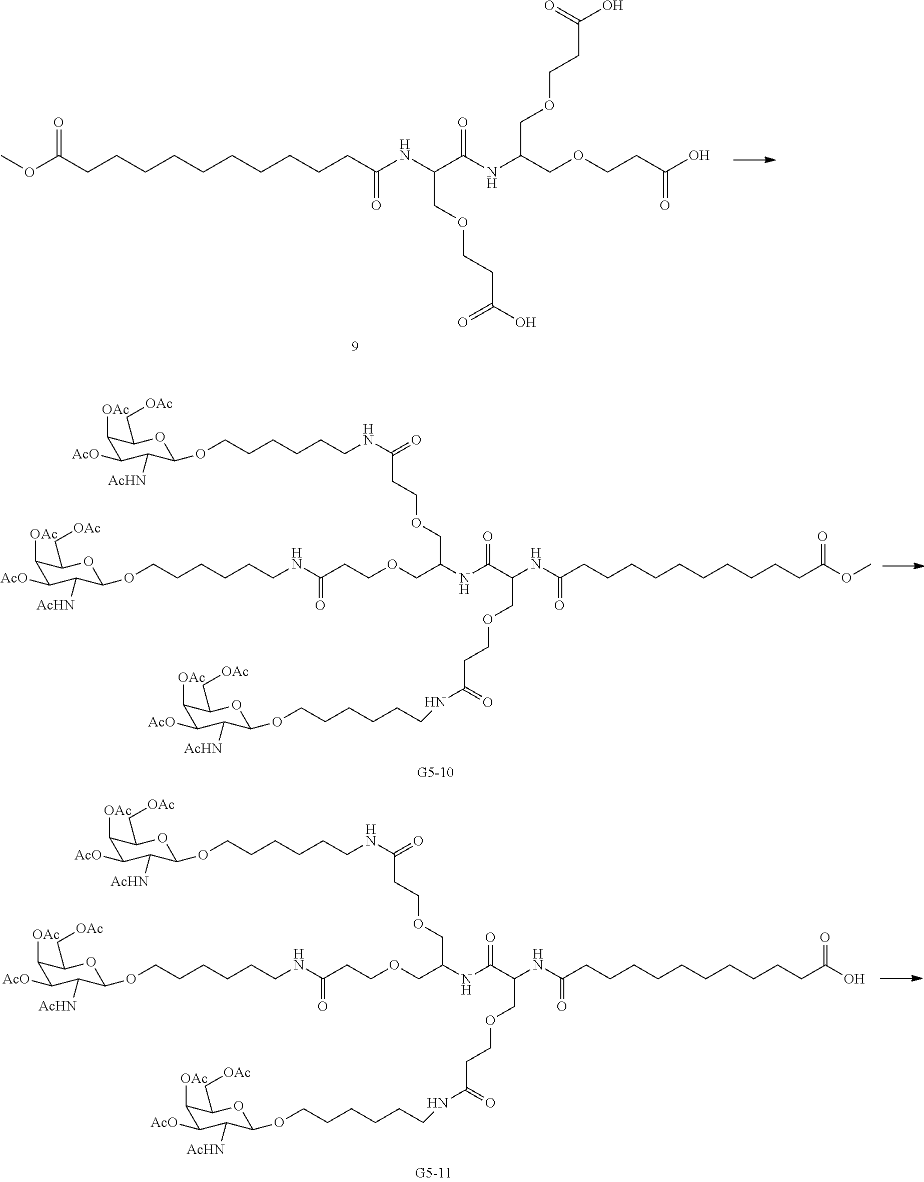 [SMILES: COC(=O)CCCCCCCCCCC(=O)NC(COCCC(=O)O)C(=O)NC(COCCC(=O)O)COCCC(=O)O ; COC(=O)CCCCCCCCCCC(=O)NC(COCCC(=O)NCCCCCCOC1OC(COC(C)=O)C(OC(C)=O)C(OC(C)=O)C1NC(C)=O)C(=O)NC(COCCC(=O)NCCCCCCOC1OC(COC(C)=O)C(OC(C)=O)C(OC(C)=O)C1NC(C)=O)COCCC(=O)NCCCCCCOC1OC(COC(C)=O)C(OC(C)=O)C(OC(C)=O)C1NC(C)=O ; CC(=O)NC1C(OCCCCCCNC(=O)CCOCC(COCCC(=O)NCCCCCCOC2OC(COC(C)=O)C(OC(C)=O)C(OC(C)=O)C2NC(C)=O)NC(=O)C(COCCC(=O)NCCCCCCOC2OC(COC(C)=O)C(OC(C)=O)C(OC(C)=O)C2NC(C)=O)NC(=O)CCCCCCCCCCC(=O)O)OC(COC(C)=O)C(OC(C)=O)C1OC(C)=O]

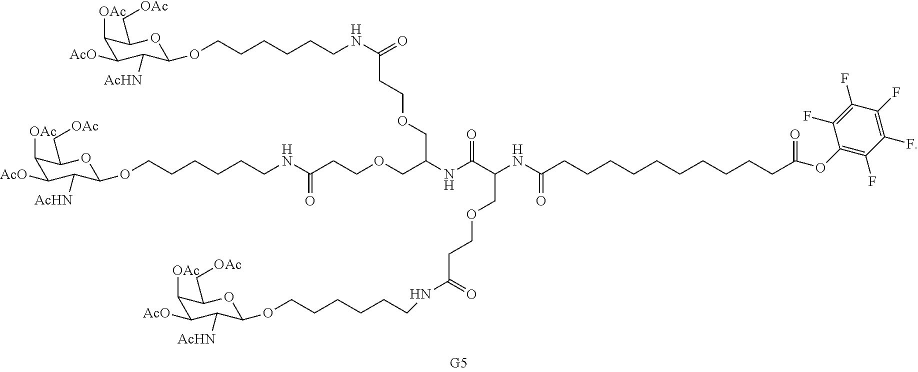 [SMILES: CC(=O)NC1C(OCCCCCCNC(=O)CCOCC(COCCC(=O)NCCCCCCOC2OC(COC(C)=O)C(OC(C)=O)C(OC(C)=O)C2NC(C)=O)NC(=O)C(COCCC(=O)NCCCCCCOC2OC(COC(C)=O)C(OC(C)=O)C(OC(C)=O)C2NC(C)=O)NC(=O)CCCCCCCCCCC(=O)Oc2c(F)c(F)c(F)c(F)c2F)OC(COC(C)=O)C(OC(C)=O)C1OC(C)=O]

Compound 9 (8 g) was dissolved in anhydrous DCM (50 mL), HOBt (1.5 eq) was added under nitrogen protection in ice bath, EDCl (1.2 eq) was added slowly, after 20 minutes, N-acetylgalactosamine (3.3 eq) and DIEA (2 eq) were added, after reaction completed, compound G5-10 was obtained by washing with saturated NaHCO₃ solution, 0.5 N HCl solution and saturated saline, concentration and chromatography, successively.

Compound G5-10 (4 g) was dissolved in anhydrous MeOH (20 mL), NaOMe (30% in MeOH, 3 mL) was added under nitrogen protection in ice bath, after reaction completed, crude product was obtained by concentration, the crude product was added in MeOH/H₂O (1:1) mixed solution and reacted at room temperature for 16 hours, Et₃N hydrochloride was used to adjust pH to neutralization, crude product was obtained by concentration. Py (20 mL) and Ac₂O (5 mL) were added and the reaction was reacted at room temperature for 24 hours, compound G5-11 (2 g) was obtained by concentration and chromatography.

Compound G5-11 (1 g) was dissolved in DCM (10 mL), EDCl (1.5 eq) and pentafluorophenol (1.5 eq) were added, after reaction completed, compound G5 (0.5 g) was obtained by washing with saturated NaHCO₃ solution, concentration and chromatography, the productivity was 40%.

¹H NMR (500 MHz, CDCl₃): δ 7.25-7.17 (m, 1H), 6.93-6.73 (m, 4H), 6.54-6.42 (m, 3H), 5.34 (d, J=2.5 Hz, 3H), 5.29-5.21 (m, 3H), 4.66 (d, J=8.2 Hz, 3H), 4.20-4.06 (m, 8H), 4.03-3.80 (m, 6H), 3.80-3.63 (m, 8H), 3.58-3.34 (m, 10H), 3.31-3.11 (m, 6H), 2.64 (t, J=7.4 Hz, 2H), 2.52-2.33 (m, 6H), 2.31-2.20 (m, 8H), 2.09-1.88 (m, 27H), 1.68-1.43 (m, 16H), 1.34 (m, 24H). ESI-Tof-MS m/z: 2057.49 [M+H]+, 2079.48 [M+Na]+; C₉₃H₁₄₁F₅N₈O₃₇.

### BIOLOGY EXPERIMENTS

### Embodiment 1: Binding Affinity Test with Hepatocyte ASGPR Receptor

Referring to Chinese Patent Application No. CN109957567B, the synthesized siRNA sequences were shown below:

(SEQ ID NO: 1)
dG*fU*dAfUdGfUfUdGfCfCfCdGfUfUfUdGfU*fC*fC,
(SEQ ID NO: 2)
mG*fG*mA*fCmAfAmAfCmGfGmGfCmAfAmCfAmUfA*mC*mC*mU.

The compound G1, G4, G5 obtained from the examples 1, 4, 5, bound to the above mentioned siRNA respectively to form conjugates which were shown in Table 1.

TABLE 1
The conjugate structure

| | Corresponding | |
|---|---|---|
| Name | siRNA No. | The conjugate structure |
| G1-12 | 1 | 5′-/G1/dG*fU*dAfUdGfUfUdGfCfCfCdGfUfUfUdGfU*fC*fC-3′(5′- |
| | | /G1/SEQ ID NO: 1-3′) |
| | 2 | 5′- |
| | | /Cy5/mG*fG*mA*fCmAfAmAfCmGfGmGfCmAfAmCfAmUfA*mC* |
| | | mC*mU-3′(5′-/Cy5/SEQ ID NO: 2-3′) |
| G4-12 | 1 | 5′-/G4/dG*fU*dAfUdGfUfUdGfCfCfCdGfUfUfUdGfU*fC*fC-3′(5′- |
| | | /G4/SEQ ID NO: 1-3′) |
| | 2 | 5′- |
| | | /Cy5/mG*fG*mA*fCmAfAmAfCmGfGmGfCmAfAmCfAmUfA*mC* |
| | | mC*mU-3′(5′-/Cy5/SEQ ID NO: 2-3′) |
| G5-12 | 1 | 5′-/G5/dG*fU*dAfUdGfUfUdGfCfCfCdGfUfUfUdGfU*fC*fC-3′(5′- |
| | | /G5/SEQ ID NO: 1-3′) |
| | 2 | 5′- |
| | | /Cy5/mG*fG*mA*fCmAfAmAfCmGfGmGfCmAfAmCfAmUfA*mC* |
| | | mC*mU-3′(5′-/Cy5/SEQ ID NO: 2-3′) |

Note:
the above A was adenine ribonucleotide, G was guanine ribonucleotide, C was cytosine ribonucleotide, U was uracil ribonucleotide, d was deoxyribonucleotide modification, f was 2′-fluoro modification, m was 2′-O-methyl modification, * was thiophosphorylated skeleton modification, Cy5 was fluorescently labeled Cy5.

Wherein, the structure of G1-12 was shown as Z₁. When G4 and G5 bound to siRNA, the phosphoester bond was needed modification, this embodiment used amino modification

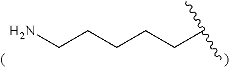 [SMILES: *CCCCCN]

of C6, so the structure of G4-12 was shown as Z₄, the structure of G5-12 was shown as Z₅.

### 1. the GalNac-siRNA Dilution

G1-12, G4-12, G5-12 (1 uM) were diluted respectively in a gradient according to the following Table 2.

TABLE 2
Substitute Specification - Clean

| | |
|---|---|
| \textcircled{1} 100 nM | 80 μL siRNA (1 μM) + 320 μL DMEM culture medium |
| | with 2% FBS |
| \textcircled{2} 50 nM | 200 μL \textcircled{1} + 200 μL DMEM culture medium |
| | with 2% FBS |
| \textcircled{3} 25 nM | 200 μL \textcircled{2} + 200 μL DMEM culture medium |
| | with 2% FBS |
| \textcircled{4} 8.3 nM | 100 μL \textcircled{3} + 200 μL DMEM culture medium |
| | with 2% FBS, 100 μL solution was abandoned after mixed |
| \textcircled{5} 2.7 nM | 100 μL \textcircled{4} + 200 μL DMEM culture medium |
| | with 2% FBS |
| \textcircled{6} 0.9 nM | 100 μL \textcircled{5} + 200 μL DMEM culture medium |
| | with 2% FBS, 100 μL solution was abandoned after mixed |

### 2. The Cell Preparation

The cell concentration was diluted by DMEM culture medium with 2% FBS to 4*10⁵ cells/mL.

### 3. The Mixing Sampling and Incubation

200 μL cells of each sample and 200 μL siRNA were mixed uniformly, 200 μL cells of the blank control group and 200 μL culture medium were mixed uniformly, and were added in 3 wells of 96-well plate with U-shaped bottom, 100 μL each well. Samples of each concentration of all siRNA were placed in the ice box after sampling, and were placed in decolorization shaker and incubated in dark for 2 hours.

### 4. Testing

50 g of 96-well plate with U-shaped bottom was centrifuged for 2 minutes at 4° C. 80 μL supernatant was aspirated and discarded by pipette, 10 μg/mL propidium iodide was prepared by using of PBS with 2% FBS, 100 μL PI reagent was added in each well and was stained for 15 minutes. After staining, 50 g of 96-well plate with U-shaped bottom was centrifuged for 2 minutes at 4° C. After washed by PBS with 2% FBS for twice, 100 μL PBS with 2% FBS was added for resuspension, flow cytometer was used to test and count the average Cy5 fluorescence intensity of live cell population.

### 5. Data Analysis

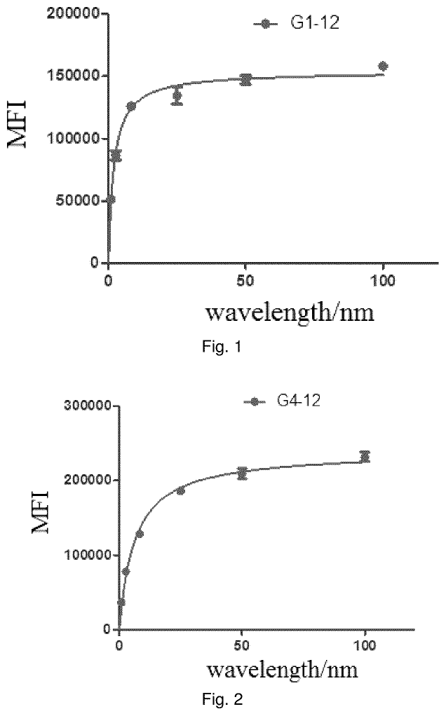

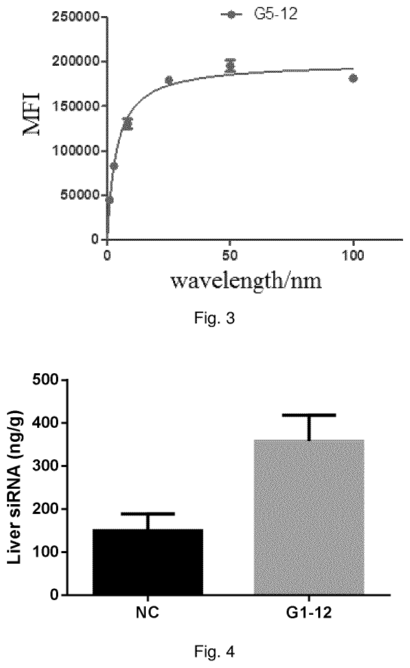

The average Cy5 fluorescence intensity of each experimental groups minus the average fluorescence intensity of blank control group, these data fitting were carried by GraphPad Prism software to draw binding saturation curves, as shown in FIGS. 1-3, the parameter was Nonlinear regression (curve fit), One site binding (hyperbola) to obtain dissociation equilibrium constant (Kd value). From Table 3, it could be known that the binding affinity between G1-12, G4-12, G5-12 and ASGPR receptor reach a higher level.

TABLE 3
the Kd value of conjugates

| Sample | G1-12 | G4-12 | G5-12 |
|---|---|---|---|
| Bmax | 153854 | 239297 | 199232 |
| Kd | 1.982 | 6.371 | 3.695 |

### Embodiment 1: The In Vivo Hepatic Targeting of GalNac-siRNA

Balb/c-nu mice, male, body weight ranged 18-22 g, were randomly divided into 3 groups, 5 mice/group, which respectively were blank control group, negative control group and targeting group, the administration dose was 100 mg/kg. The negative control group was administered by tail vein injection of negative control test substance (NC, Ribobio, siM200922112438) once, the targeting group was administered by tail vein injection of the obtained G1-12 test substance of example 6 once, the blank control group was administered an equal volume of saline. All animals' livers were immediately placed in liquid nitrogen after 6 hours of administration, then were saved at −80° C., the siRNA pharmaceutical concentration of hepatic tissue was tested by qPCR method.
1. Drawing the standard curve. Referring to the specification of siRNA antisense standard (Ribobio, ssR190807024906), the standard sample mother liquor was prepared, the concentration of siRNA was 500 nM. The standard sample mother liquor was diluted according to 10 times gradient (50 nM-0.05 μM), 7 gradients in total. Samples were placed at 95° C. and incubated for 10 minutes, and precooled Rtmix was added quickly for reverse transcription and qPCR. The reverse transcription was performed with reference to the specification of BpLge-Loop™ miRNA qRT-PCR Starter Kit (Ribobio, C10211-2), the qPCR was performed with reference to the specification of riboSCRIPT™ Reverse Transcription Kit (Ribobio, C11027-2). The average Ct values of each standard points were calculated based on the obtained Ct values, the log value of standard point concentration was abscissa, the obtained average Ct value was ordinate, the standard curve was drawn, the regression equation and R² were calculated, R²>0.99.
2. Handling the sample. The mice liver samples were ground to powder by liquid nitrogen grinding method, and the weight of powder was measured. Based on the weight, the corresponding volume of preheated 0.25% Triton X-100 (100 mg/ml) at 95° C. was added, swirled and oscillated. The samples were placed at 95° C. and incubated for 10 minutes after mixing. 20000 g of the lysis buffer was placed on the ice to cool down and was centrifuged at 4° C. for 20 minutes. The supernatant was collected. The supernatant was diluted 10000 times with Rnase free water, 20 μL of the dilution solution was placed in PCR instrument at 95° C. and incubated for 10 minutes, the precooled Rtmix was added quickly for reverse transcription and qPCR. The reverse transcription was performed with reference to the specification of BpLge-Loop™ miRNA qRT-PCR Starter Kit (Ribobio, C10211-2), the qPCR was performed with reference to the specification of riboSCRIPT™ Reverse Transcription Kit (Ribobio, C11027-2).
3. Data analysis. The sample Ct values were substituted into the regression equation of the standard curve, the siRNA content of samples was calculated.

From FIG. 4, compared with the negative control siRNA that did not link to the GalNAc, the distribution of G1-12 of GalNAc-siRNA structure was obviously increased in mice liver, showing that G1-12 could achieve hepatic targeting delivery in vivo.

### Embodiment 3: Know Down Effect to the Expression of PCSK9 mRNA

Referring to Chinese Patent Application No. CN109957567B, the synthesized siRNA sequences were shown below:

(SEQ ID NO: 3)
mG*mG*mUmCmUmGfGfAfAmUmGmCmAmAmAmG*mU*mC*mA,
(SEQ ID NO: 4)
mU*fG*mAfCmUfUmUfGmCfAmUfUmCfCmAfG*mA*mC*mC.

The compound G2, G4, G5 obtained from the examples 2, 4, 5, bound to the above mentioned siRNA respectively to form conjugates which were shown in Table 4.

TABLE 4
the conjugate structure

| | Corresponding | |
|---|---|---|
| Name | siRNA No. | The conjugate structure |
| G2-34 | 3 | 5′- |
| | | /G2/mG*mG*mUmCmUmGfGfAfAmUmGmCmAmAmAmG*m |
| | | U*mC*mA-3′ (5′-/G2/SEQ ID NO: 3-3′) |
| | 4 | 5′- |
| | | phos*/mU*fG*mAfCmUfUmUfGmCfAmUfUmCfCmAfG*mA*m |
| | | C*mC-3′ (5′-phos*/SEQ ID NO: 4-3′) |
| G4-34 | 3 | 5′- |
| | | /G4/mG*mG*mUmCmUmGfGfAfAmUmGmCmAmAmAmG*m |
| | | U*mC*mA-3′ (5′-/G4/SEQ ID NO: 3-3′) |
| | 4 | 5′- |
| | | phos*/mU*fG*mAfCmUfUmUfGmCfAmUfUmCfCmAfG*mA*m |
| | | C*mC-3′ (5′-phos*/SEQ ID NO: 4-3′) |
| G5-34 | 3 | 5′- |
| | | /G5/mG*mG*mUmCmUmGfGfAfAmUmGmCmAmAmAmG*m |
| | | U*mC*mA-3′ (5′-/G5/SEQ ID NO: 3-3′) |
| | 4 | 5′- |
| | | phos*/mU*fG*mAfCmUfUmUfGmCfAmUfUmCfCmAfG*mA*m |
| | | C*mC-3′ (5′-phos*/SEQ ID NO: 4-3′) |

Note:
the above A was adenine ribonucleotide, G was guanine ribonucleotide, C was cytosine ribonucleotide, U was uracil ribonucleotide, f was 2′-fluoro modification, m was 2′-O-methyl modification, * was thiophosphorylated skeleton modification, 5′-phos* was phosphorothioate of 5′ end.

Wherein, the structure of G2-34 was shown as Z₂. When G4 and G5 bound to siRNA, the phosphoester bond was needed modification, this embodiment used amino modification

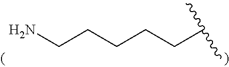 [SMILES: *CCCCCN]

of C6, so the structure of G4-34 was shown as Z₄, the structure of G5-34 was shown as Z₅.

Hep3B cell (ATCC, HB-8064) was cultured, the cell density was about 30% to 50% before transfection and relatively uniformly.

Each conjugate samples of Table 4 were prepared to 20 μM storage solution, and were set as experimental groups. Except the experimental groups, normal cell control group (Blank), transfection reagent control group (Mock), negative control group 1 (NC001, Ribobio, siM21 1215011852) and negative control group 2 (NC002, Ribobio, siM21 1215011852) were also set. Referring to the specification of Lipofectamine™ 3000 transfection reagent (Thermo Fisher, L300001), cells were transfected. Both the experimental groups and the control groups had 3 replicates.

The total RNA extraction was performed with reference to the specification of MagZol Reagent (Magen, R4801).

The reverse transcription was performed with reference to the specification of riboSCRIPT™ Reverse Transcription Kit (Ribobio, C11027-2).

Human house keeping gene actin was used for reference gene, forward primer sequence of actin gene: 5′-TCAAGATCATTGCTCCTCCTGAG-3′ (SEQ ID NO: 5), reverse primer sequence: 5′-ACATCTGCTGGAAGGTGGACA-3′ (SEQ ID NO: 6); forward primer sequence of human PCSK9 gene: 5′-AAGCCAAGCCTCTTCTTACTTCA-3′ (SEQ ID NO: 7), reverse primer sequence: 5′-CCTGGGTGATAACGGAAAAAG-3′ (SEQ ID NO: 8). 2×SYBR Green Mix (Ribobio, C10712F −1 m l) and fluorescence quantitative PCR instrument (BioRad, CFX96) were used to perform real time fluorescence quantitative PCR reaction. After PCR reaction, the Ct deviation of 9 replicates of a sample (3 transfection replicates×3 qPCR duplicates) should be ±0.5. Then CFX 2.1 software was used for quantitative analysis.

As shown in Table 5, the knock down efficiency to target gene PCSK9 of the conjugates formed by different GalNAc structures and same siRNA sequence in Hep3B cell reached about 60%, which can obviously knock down the expression of target gene mRNA.

TABLE 5
PCSK9 mRNA relative expression value

| | The average |
|---|---|
| Sample name | relative expression |
| NC001 | 1.00 |
| NC002 | 0.87 |
| Blank | 0.98 |
| Mock | 0.97 |
| G2-34 | 0.34 |
| G4-34 | 0.41 |
| G5-34 | 0.33 |

Although the embodiments of the present disclosure have been shown and described, it is apparent to those skilled in the art that a variety of changes, modifications, substitutions, and replacements may be made without departing from the scope of the present disclosure. The scope of the present disclosure is defined by the appended claims and their equivalents.

1. A compound of formula (I), or a pharmaceutical acceptable salt, tautomer or stereoisomer thereof: 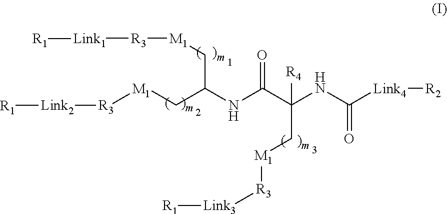
wherein,
M₁ is independently selected from at least one of —O—, —S—, —NR₆—, —CHR₇—, —C(O)—, —S(O)— and —S(O)₂—;
R₁ is residue of asialoglycoprotein receptor (ASGPR) ligand;
R₂ is at least one selected from hydroxyl, carboxyl, —ORₚ, —OPG₂, 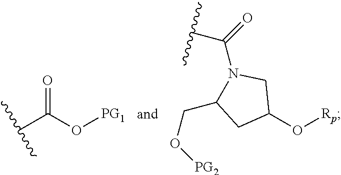
PG₁ is carboxyl protecting group;
PG₂ is hydroxyl protecting group;
Rₚ is 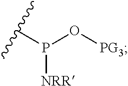
PG₃ is independently selected from phosphate protecting group;
R and R′ are respectively and independently selected from amino protecting group; or R, R′ and the N atom they linked optionally form together to be a 5-10 membered heterocyclic group;
R₃ is independently selected from 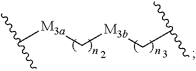
M₃ₐ is independently selected from at least one of chemical bond, —O—, —S—, —NR₆—, —CHR₇—, —C(O)—, —S(O)₁₋₂—, —NR₆C(O)—, —C(O)NR₆—, —NR₆S(O)₁₋₂—, —S(O)₁₋₂NR₆—, —OC(O)—, —C(O)O—, —OS(O)₁₋₂— and —S(O)₁₋₂O—;
M_{3b} is independently selected from at least one of —O—, —S—, —NR₆—, —CHR₇—, —CH═CH—, —C≡C—, —C(O)—, —S(O)₁₋₂—, —C(O)NR₆—, —NR₆C(O)—, —OC(O)—, —C(O)O—, —S(O)₁₋₂₀—Y₁—, —OS(O)₁₋₂—Y₁—, —OS(O)₁₋₂O—, —S(O)₁₋₂NR₆—Y₁—, —NR₆S(O)₁₋₂—Y₁—, C₆₋₁₀ arylene, 5-10 membered heteroarylene, 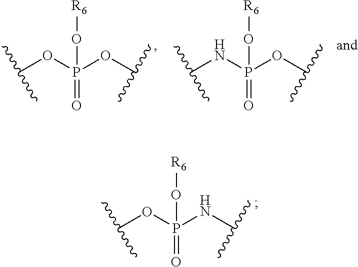
Y₁ is independently selected from C₆₋₁₀ arylene or 5-10 membered heteroarylene;
R₆ is respectively and independently selected from at least one of H, C₁₋₁₀ alkyl, C₁₋₁₀ haloalkyl, C₃₋₁₀ cycloalkyl, 3-10 membered heterocyclyl;
R₇ is respectively and independently selected from at least one of H, halogen, —C≡N, —NO₂, C₁₋₁₀ alkyl, C₁₋₁₀ haloalkyl, C₃₋₁₀ cycloalkyl, 3-10 membered heterocyclyl, C₆₋₁₀ arylene, 5-10 membered heteroaryl group, wherein, one or multiple methylene of the said alkyl or haloalkyl is optionally and independently substituted by the following group which is selected from at least one of —C(O)—, —C(O)NH—, —NHC(O)—, —OC(O)—, —O—, —C(O)O—, —S—, —S—(CH₂)₀₋₆—S—, —NR₆—, C₆₋₁₀ arylene or 5-10 membered heteroarylene, —CH═CH—, —C≡C— and —O—(CH₂)₁₋₆—O—;
n₂ is independently selected from 0, 1, 2, 3, 4, 5, 6, 7, 8, 9 or 10;
n₃ is independently selected from 1, 2, 3, 4, 5 or 6;
R₄ is selected from at least one of H, C₁₋₆ alkyl, C₁₋₆ haloalkyl and C₁₋₆ alkoxy;
Link₁, Link₂, Link₃ and Link₄ are respectively and independently selected from C₁₋₂₅ linear chain alkylene, wherein, one or multiple methylene of the said linear chain alkylene is optionally and independently substituted by R*, R* is independently selected from at least one of —O—, —S—, —NR₆—, —CHR₇—, —C(O)—, —S(O)₁₋₂—, —NR₆C(O)—, —C(O)NR₆—, —NR₆S(O)₁₋₂—, —S(O)₁₋₂NR₆—, —OC(O)—, —C(O)O—, —OS(O)₁₋₂— and —S(O)₁₋₂O—;
m₁, m₂ and m₃ are respectively and independently selected from 1, 2, 3, 4 or 5;
with the proviso that the methylene connected to the carbonyl of left side in Link₄ is not substituted by R*.

2. The formula (I) compound of claim 1, or the pharmaceutical acceptable salt, tautomer or stereoisomer thereof, wherein, M₁ is independently selected from at least one of —O—, —S—, —NH—, —CH₂—, —C(O)—, —S(O)— and —S(O)₂—, preferably selected from at least one of —O—, —S— and —NH—, preferably —O—;
and/or R₁ is independently selected from at least one of residue of the following structure: D-mannopyranose, L-mannopyranose, L-arabinose, D-arabinose, D-xylofuranose, L-xylofuranose, D-glucose, L-glucose, D-galactose, L-galactose, α-D-mannofuranose, β-D-mannofuranose, α-D-mannopyranose, Q-D-mannopyranose, α-D-glucopyranose, β-D-glucopyranose, α-D-glucofuranose, β-D-glucofuranose, α-D-fructofuranose, β-D-fructofuranose, α-D-fructopyranose, β-D-fructopyranose, α-D-galactopyranose, β-D-galactopyranose, α-D-galactofuranose, β-D-galactofuranose, glucosamine, sialic acid, galactosamine, N-acetylgalactosamine, N-trifluoroacetylgalactosamine, N-propionylgalactosamine, N-n-butyrylgalactosamine, N-isobutyrylgalactosamine, 2-amino-3-O—[(R)-1-carboxyethyl]-2-deoxy-β-D-glucopyranose, 2-deoxy-2-methylamino-L-glucopyranose, 4,6-dideoxy-4-carboxamido-2,3-di-O-methyl-D-mannopyranose, 2-deoxy-2-sulfamino-D-glucopyranose, N-glycolyl-α-neuraminic acid, 5-thio-β-D-glucopyranose, 2,3,4-tri-O-acetyl-1-thio-6-O-tribenzyl-α-D-glucopyranosyl methyl ester, 4-thio-(-D-galactopyranose, 3,4,6,7-tetra-O-acetyl-2-deoxy-1,5-dithio-α-D-glucoheptoside ethyl ester, 2,5-anhydro-D-allosenitrile, ribose, D-ribose, D-4-thioribose, L-ribose and L-4-thioribose;
preferably, the R₁ is independently selected from at least one of residue of the following structure: D-galactose, L-galactose, α-D-galactopyranose, β-D-galactopyranose, α-D-galactofuranose, β-D-galactofuranose, galactosamine, N-acetylgalactosamine, N-trifluoroacetylgalactosamine, N-propionylgalactosamine, N-n-butyrylgalactosamine, N-isobutyrylgalactosamine and β-D-galactofuranose, preferably selected from at least one of galactosamine, N-acetylgalactosamine, N-trifluoroacetylgalactosamine, N-propionylgalactosamine, N-n-butyrylgalactosamine, N-isobutyrylgalactosamine, preferably N-acetylgalactosamine;
preferably, R₁ is 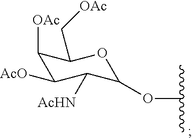 [SMILES: *OC1OC(COC(C)=O)C(OC(C)=O)C(OC(C)=O)C1NC(C)=O]
and/or R₂ is selected from at least one of hydroxyl, carboxyl, —ORₚ, —OPG₂, 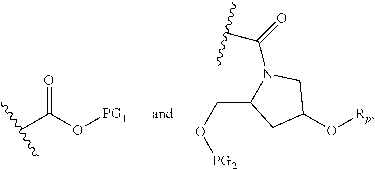
preferably the R₂ is selected from at least one of —ORₚ, 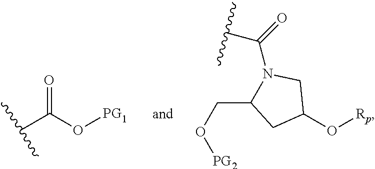
preferably the R₂ is selected from 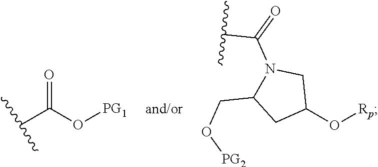
or preferably the R₂ is selected from at least one of 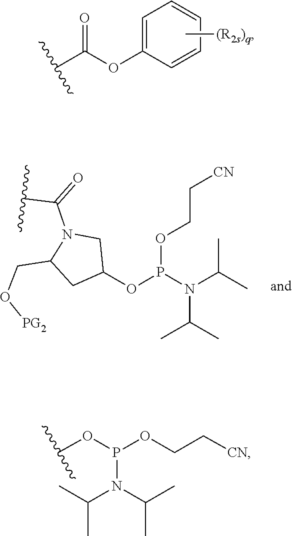
preferably selected from 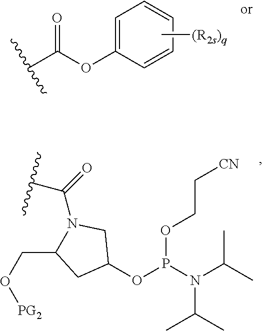
preferably 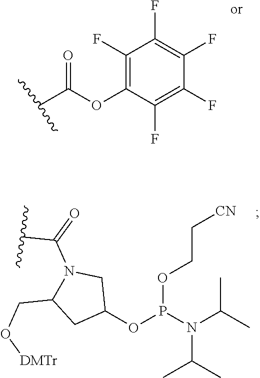
preferably, 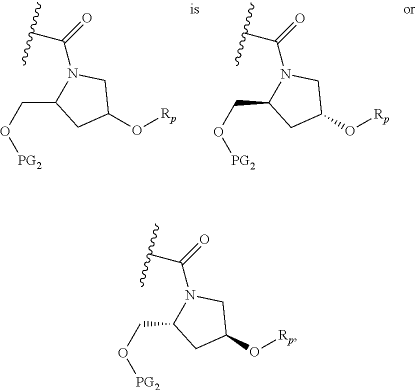
preferably

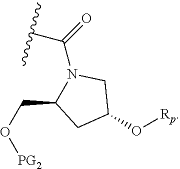

3. (canceled)

4. (canceled)

5. The formula (I) compound of claims 1-4, or the pharmaceutical acceptable salt, tautomer or stereoisomer thereof, wherein, PG₁ is selected from at least one of C₁₋₆ alkyl, benzyl, allyl, 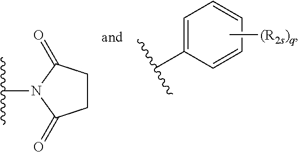
wherein, R₂ₛ is independently selected from at least one of H, halogen, C₁₋₁₀ alkyl, C₁₋₁₀ haloalkyl, q is 0, 1, 2, 3, 4 or 5;
preferably, PG₁ is selected from at least one of methyl, tert-butyl group, benzyl, allyl, 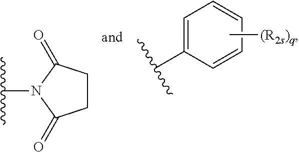
preferably the PG₁ is selected from at least one of 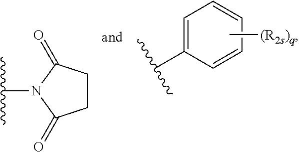
preferably the PG₁ is 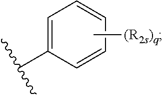
preferably, R₂ₛ is independently selected from at least one of H, halogen, C₁₋₆ alkyl and C₁₋₆ haloalkyl, preferably H or halogen, preferably —F, preferably the PG₁ is 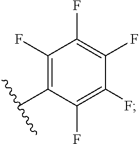 [SMILES: *c1c(F)c(F)c(F)c(F)c1F]
and/or PG₂ is selected from at least one of trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethyloxycarbonyl (Fmoc), allyloxycarbonyl (Alloc), 2,2,2-trichloroethoxycarbonyl (Troc), carbobenzyloxy (Cbz), tert-butyloxycarbonyl (Boc), benzyl (Bn), p-methoxybenzyl (PMB), allyl, dimethoxytrityl (DMT), monomethoxytrityl (MMT), 9-phenyloxaanthracen-9-yl (Pixyl), 9-(p-methoxyphenyl) oxaanthracen-9-yl (Mox), trityl (Tr), 4-methoxytriphenylmethyl (MMTr), 4,4′-dimethoxytrityl (DMTr), 4,4′,4″-trimethoxytriphenylmethyl (TMTr), methoxymethyl (MOM), phenoxymethyl (BOM), 2,2,2-trichloroethoxymethyl, 2-methoxyethoxymethyl (MEM), methylthiomethyl (MTM), and p-methoxybenzyloxymethyl (PMBM);
preferably, PG₂ is selected from at least one of dimethoxytrityl (DMT), monomethoxytrityl (MMT), 9-phenyloxaanthracen-9-yl (Pixyl), 9-(p-methoxyphenyl) oxaanthracen-9-yl (Mox), trityl (Tr), 4-methoxytriphenylmethyl (MMTr), 4,4′-dimethoxytrityl (DMTr), tert-butyldimethylsilyl (TBDMS), tert-butyldiphenylsilyl (TBDPS), triisopropylsilyl (TIPS), preferably selected from at least one of dimethoxytrityl (DMT), monomethoxytrityl (MMT), trityl (Tr), 4-methoxytriphenylmethyl (MMTr), 4,4′-dimethoxytrityl (DMTr), and 4,4′,4″-trimethoxytriphenylmethyl (TMTr), preferably 4,4′-dimethoxytrityl (DMTr);
and/or PG₃ is independently selected from at least one of 2-cyanoethyl and C₁₋₆ alkyl, preferably the PG₃ is independently selected from at least one of 2-cyanoethyl and methyl, preferably 2-cyanoethyl;
and/or R and R′ are respectively and independently selected from C₁₋₆ alkyl, preferably isopropyl;
and/or the R, the R′ and the N atom they linked optionally form together to be a 5-6 membered heterocyclic group.

6. (canceled)

7. (canceled)

8. (canceled)

9. (canceled)

10. The formula (I) compound of claim 1, or the pharmaceutical acceptable salt, tautomer or stereoisomer thereof, wherein, Rₚ is 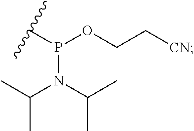 [SMILES: *P(OCCC#N)N(C(C)C)C(C)C]
and/or M₃ₐ is independently selected from at least one of chemical bond, —O—, —S—, —NR₆—, —CHR₇—, —C(O)—, —S(O)₁₋₂—, —NHC(O)—, —C(O)NH—, —NHS(O)₁₋₂—, —S(O)₁₋₂NH—, —OC(O)—, —C(O)O—, —OS(O)₂— and —S(O)O₂—, preferably the M₃ₐ is independently selected from at least one of chemical bond, —O—, —S—, —NR₆—, —C(O)—, —NHC(O)—, —C(O)NH—, —OC(O)— and —C(O)O—, preferably the Ma is independently selected from at least one of chemical bond, —NHC(O)—, —C(O)NH—, —OC(O)— and —C(O)O—, preferably the M₃ₐ is independently selected from at least one of chemical bond, —NHC(O)— and —C(O)NH—, preferably the M₃ₐ is independently chemical bond or —C(O)NH—;
and/or M_{3b} is independently selected from at least one of —O—, —S—, —NR₆—, —CHR₇—, —CH═CH—, —C≡C—, —C(O)—, —S(O)₁₋₂—, —C(O)NH—, —NHC(O)—, —OC(O)—, —C(O)O—, —S(O)₁₋₂O—Y₁—, —OS(O)₁₋₂—Y₁—, —OS(O)₁₋₂O—, —S(O)₁₋₂NH—Y₁—, —NHS(O)₁₋₂—Y₁—, phenylene, 5-6 membered heteroarylene, 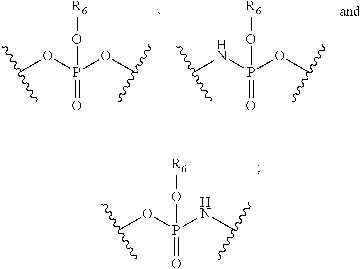
preferably, the M_{3b} is independently selected from at least one of —O—, —S—, —NR₆—, —CHR₇—, —CH═CH—, —C≡C—, —C(O)—, —C(O)NH—, —NHC(O)—, —OC(O)—, —C(O)O—, —OS(O)₂—Y₁—, —OS(O)₂O—, —NHS(O)₂—Y₁—, 5-6 membered heteroarylene, 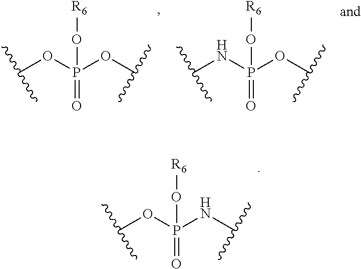
preferably the M_{3b} is independently selected from at least one of —C(O)NH—, —NHC(O)—, —OC(O)—, —C(O)O—, preferably the M_{3b} is independently —C(O)NH— or —NHC(O)—, preferably the M_{3b} is independently —NHC(O)—;
preferably, the 5-6 membered heteroarylene of the M_{3b} is 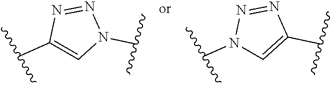 [SMILES: *c1cn(*)nn1 ; *c1cn(*)nn1]
preferably, Y₁ is independently selected from phenylene, 5-6 membered heteroarylene, preferably phenylene, preferably

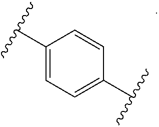 [SMILES: *c1ccc(*)cc1]

11. (canceled)

12. (canceled)

13. The formula (I) compound of claim 1, or the pharmaceutical acceptable salt, tautomer or stereoisomer thereof, wherein, 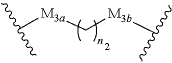
is selected from at least one of the following structure: 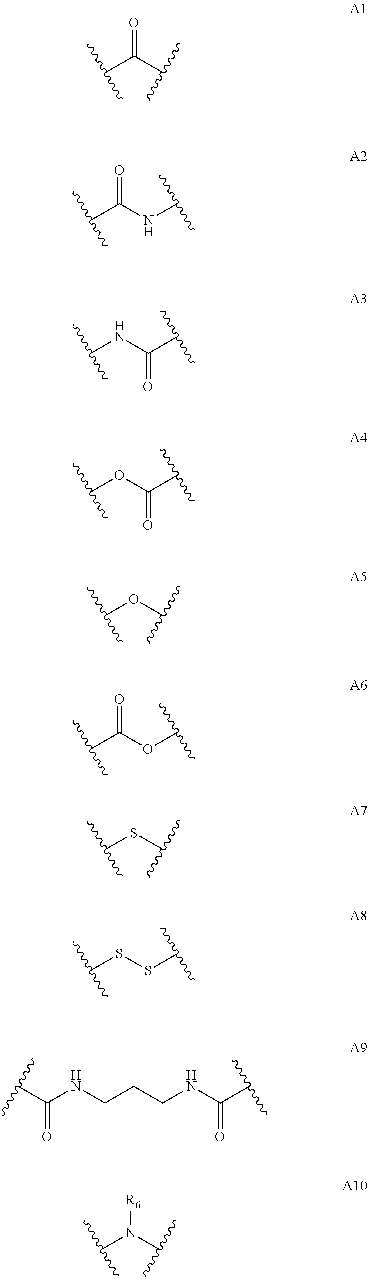 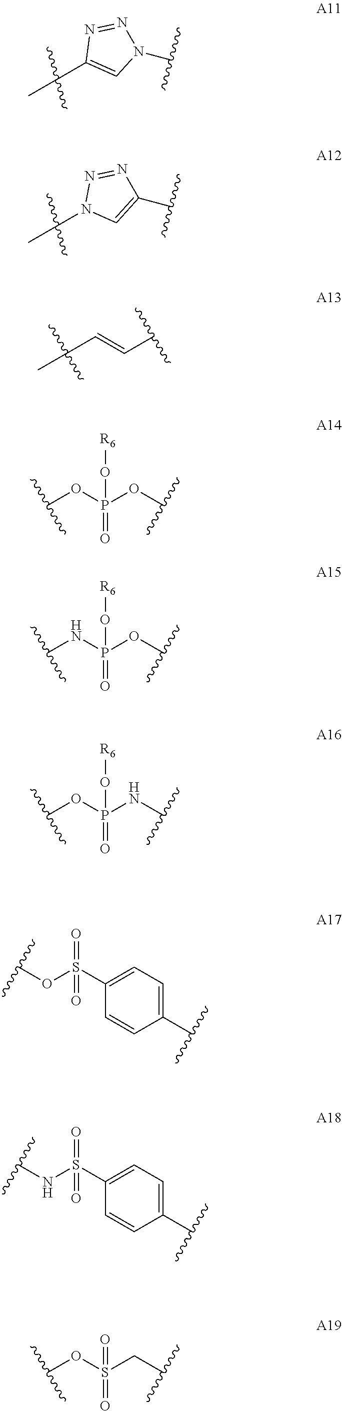 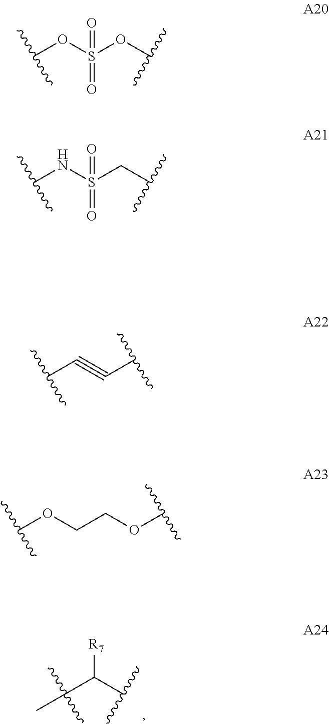
preferably the 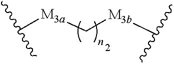
is selected from at least one of A2, A3, A4, A6 and A9, preferably the 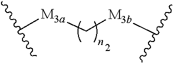
is selected from at least one of A2, A3, A9, preferably the 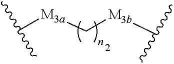
is A3 or A9;
and/or R₆ is independently selected from at least one of H, C₁₋₆ alkyl, C₁₋₆ haloalkyl, C₃₋₇ cycloalkyl and 3-7 membered heterocyclyl, preferably the R₆ is independently selected from at least one of H, C₁₋₆ alkyl, and C₁₋₆ haloalkyl preferably the R₆ is independently selected from at least one of H, —CH₃, 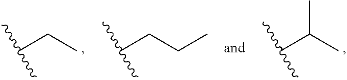
more preferably the R₆ is independently H;
and/or R₇ is independently selected from at least one of H, halogen, —C≡N, C₁₋₆ alkyl, C₁₋₆ haloalkyl, C₃ cycloalkyl, 3-7 membered heterocyclyl, phenyl, and 5-6 membered heteroaryl, preferably the R₇ is independently selected from at least one of H, halogen, —C≡N, C₁₋₆ alkyl and C₁₋₆ haloalkyl, preferably the R₇ is independently selected from at least one of H, halogen, C₁₋₆ alkyl and C₁₋₆ haloalkyl;
preferably, one or multiple methylene of the said alkyl or haloalkyl of R₇ are optionally and independently substituted by the following groups which are selected from at least one of —C(O)—, —C(O)NH—, —NHC(O)—, —OC(O)—, —O—, —C(O)O—, —S—, —S—(CH₂)₀₋₃—S—, —NR₆—, phenylene, 5-6 membered heteroarylene, —CH═CH—, —C≡C— and —O—(CH₂)₁₋₃—O—, preferably selected from at least one of —C(O)—, —C(O)NH—, —NHC(O)—, —OC(O)—, —O—, —C(O)O—, —S—, —S—S—, —NR₆, 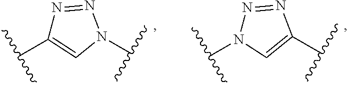 [SMILES: *c1cn(*)nn1 ; *c1cn(*)nn1]
—CH═CH—, —C≡C— and —O—(CH₂)₂—O—;
preferably, the halogen of the R₂ is —F, —Cl or —Br.

14. (canceled)

15. (canceled)

16. The formula (I) compound of claim 1, or the pharmaceutical acceptable salt, tautomer or stereoisomer thereof, wherein, n₂ is independently 0, 1, 2, 3, 4, 5 or 6, preferably 0, 1, 2 or 3, preferably 0 or 3;
n₃ is independently 1, 2, 3, preferably 2.

17. (canceled)

18. The formula (I) compound of claim 1, or the pharmaceutical acceptable salt, tautomer or stereoisomer thereof, wherein, R₄ is selected from at least one of H, C₁₋₅ alkyl, C₁₋₅ haloalkyl and C₁₋₅ alkoxy, preferably the R₄ is selected from at least one of H, C₁₋₃ alkyl and C₁₋₃ haloalkyl, preferably the R₄ is H;
and/or Link₁, Link₂ and Link₃ are respectively and independently selected from C₁₋₁₀ linear chain alkylene, preferably C₁₋₆ linear chain alkylene, preferably C₄₋₆ linear chain alkylene, preferably —(CH₂)₄— or —(CH₂)₆—;
and/or Link₄ is C₁₋₁₀ linear chain alkylene, preferably C₁₇ linear chain alkylene, preferably C₁₋₁₀ linear chain alkylene, preferably C₃₋₂₀ linear chain alkylene, preferably C₃₋₁₇ linear chain alkylene, preferably C₃₋₁₀ linear chain alkylene;
preferably, one or multiple methylenes of the linear chain alkylene of Link₄, preferably 1, 2, or 3 methylenes, preferably 1 methylene is optionally and independently substituted by R*;
preferably, R* is independently selected from at least one of —O—, —NR₆—, —CHR₇—, —C(O)—, —NR₆C(O)—, —C(O)NR₆—, —OC(O)— and —C(O)O—, preferably at least one of —C(O)NH—, —NHC(O)—, —C(O)O— and —OC(O)—, preferably at least one of —C(O)NH— and —C(O)O—, or preferably at least one of —C(O)NH— and —NHC(O)—, more preferably —C(O)NH—;
preferably, R* is separated from the carbonyl on the left side of Link₄ by 1-15 methylenes, preferably 1-12 methylenes, preferably 1-10 methylenes, preferably 1-3 methylenes, preferably 3-10 methylenes;
R is separated from R₂ by 1-12 methylenes, preferably 1-10 methylenes, preferably 1-6 methylenes;
preferably, Link₄ is selected from at least one of —(CH₂)₃—, —(CH₂)₁₀—, —(CH₂)₃—C(O)NH—(CH₆)₆— and —(CH₂)₁₀—C(O)NH—(CH₂)₆—, preferably selected from at least one of —(CH₃)₃—, —(CH₂)₁₀— and —(CH₂)₃—C(O)NH—(CH₂)₆—, preferably —(CH₂)₃— or —(CH₂)₁₀—; or preferably selected from at least one of —(CH₂)₁₀—, —(CH₂)₃—C(O)NH—(CH₂)₆— and —(CH₂)₁₀—C(O)NH—(CH₂)₆—, preferably —(CH₂)₁₀— or —(CH₂)₁₀—C(O)NH—(CH₂)₆—;
and/or m₁, m₂ and m₃ are respectively and independently 1, 2 or 3, preferably 1.

19. (canceled)

20. (canceled)

21. (canceled)

22. The formula (I) compound of claim 1, or the pharmaceutical acceptable salt, tautomer or stereoisomer thereof, which have the following structures: 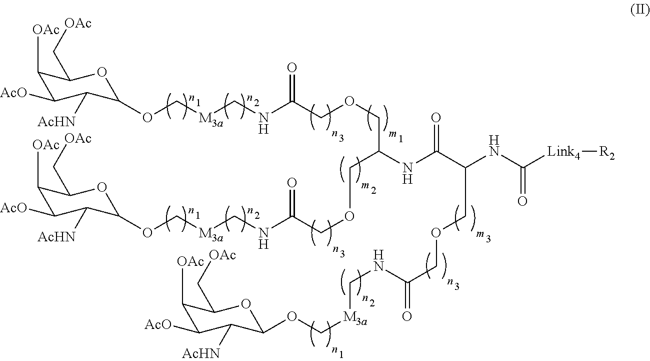
wherein,
n₂ is independently 0, 1, 2, 3, 4, 5, 6, 7, 8, 9 or 10;
each remaining groups are as defined in claim 1;
and/or R₂ is selected from at least one of hydroxyl, carboxyl, —ORₚ, —OPG₂, 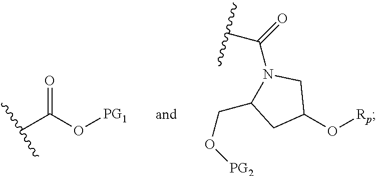
PG₁ is carboxyl protecting group, preferably selected from at least one of methyl, tert-butyl group, benzyl, allyl, 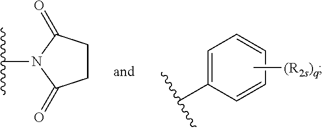
R₂, is independently selected from at least one of H, halogen, C₁₋₆ alkyl and C₁₋₆ haloalkyl;
q is 0, 1, 2, 3, 4 or 5;
PG₂ is hydroxyl protecting group, preferably selected from at least one of dimethoxytrityl (DMT), monomethoxytrityl (MMT), 9-phenyloxaanthracen-9-yl (Pixyl), 9-(p-methoxyphenyl) oxaanthracen-9-yl (Mox), trityl (Tr), 4-methoxytriphenylmethyl (MMTr), 4,4′-dimethoxytrityl (DMTr), 4,4′,4″-trimethoxytriphenylmethyl (TMTr), tert-butyldimethylsilyl (TBDMS), tert-butyldiphenylsilyl (TBDPS), and triisopropylsilyl (TIPS);
Rₚ is 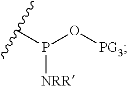
PG₃ is independently selected from at least one of phosphate protecting group, preferably selected from 2-cyanoethyl and methyl, more preferably 2-cyanoethyl;
R and R′ are respectively and independently amino protecting group, preferably C₁₋₆ alkyl, preferably isopropyl;
M₃ₐ is selected from at least one of chemical bond, —NHC(O)—, —C(O)NH—, —OC(O)— and —C(O)O—, preferably the M₃ₐ is selected from at least one of chemical bond, —NHC(O)—, and —C(O)NH—;
n₁ is independently 1, 2, 3, 4, 5, 6, 7, 8, 9 or 10;
n₂ is independently 0, 1, 2, 3, 4, 5 or 6;
n₃ is independently 1, 2, 3, 4, 5 or 6;
m₁, m₂ and m₃ are independently 1, 2, 3, 4 or 5;
Link₄ is selected from at least one of C₁₋₂₅ linear chain alkylene, preferably C₁₋₂₀ linear chain alkylene, wherein, 1, 2 or 3 methylenes of the said linear chain alkylene are optionally and independently substituted by R*, R* is independently selected from: —C(O)NH—, —NHC(O)—, —C(O)O— and —OC(O)—, preferably at least one of —C(O)NH— and —NHC(O)—;
with the proviso that the methylene connected to the carbonyl of left side in Link₄ is not substituted by R*.

23. (canceled)

24. The formula (II) compound of claim 22, or the pharmaceutical acceptable salt, tautomer or stereoisomer thereof, wherein, R₂ is selected from at least one of —ORₚ 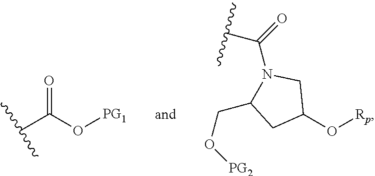
preferably selected from at least one of 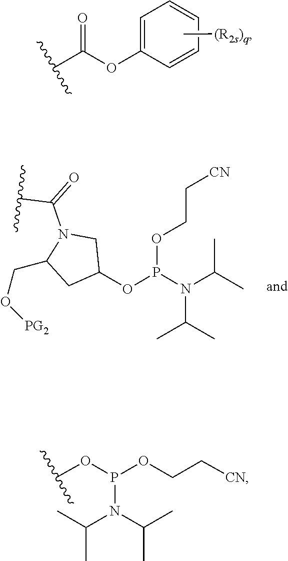
preferably 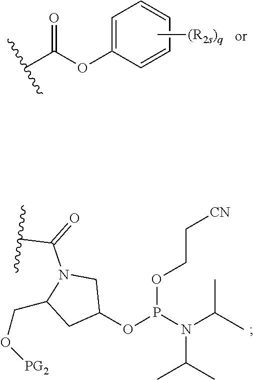
PG₁ is selected from at least one of 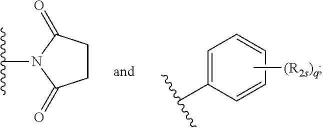
R₂ₛ is independently H or halogen, preferably —F;
q is 0, 1, 2, 3, 4 or 5;
PG₂ is selected from at least one of dimethoxytrityl (DMT), monomethoxytrityl (MMT), trityl (Tr), 4-methoxytriphenylmethyl (MMTr), 4,4′-dimethoxytrityl (DMTr), and 4,4′,4″-trimethoxytriphenylmethyl (TMTr), preferably the PG₂ is 4,4′-dimethoxytrityl (DMTr);
Rₚ is 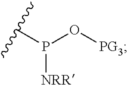
PG₃ is 2-cyanoethyl;
R and R′ are respectively and independently C₁₋₄ alkyl, preferably isopropyl;
M₃ₐ is chemical bond or —C(O)NH—;
n₁ is independently 4, 5 or 6;
n₂ is independently 0, 1, 2 or 3;
n₃ is independently 1 or 2;
m₁, m₂ and m₃ are respectively and independently 1, 2 or 3, preferably 1;
Link₄ is selected from C₁₋₁₇ linear chain alkylene, preferably C₁₋₁₀ linear chain alkylene, wherein, one methylene of the said linear chain alkylene is optionally substituted by R*, R* is —C(O)NH—;
preferably, R* is separated from the carbonyl on the left side of Link₄ by 1-12 methylenes, preferably 1-10 methylenes, preferably 1-3 methylenes;
R* is separated from R₂ by 1-12 methylenes, preferably 1-10 methylenes, preferably 1-6 methylenes.

25. The formula (II) compound of claim 24, or the pharmaceutical acceptable salt, tautomer or stereoisomer thereof, wherein, R₂ is selected from at least one of 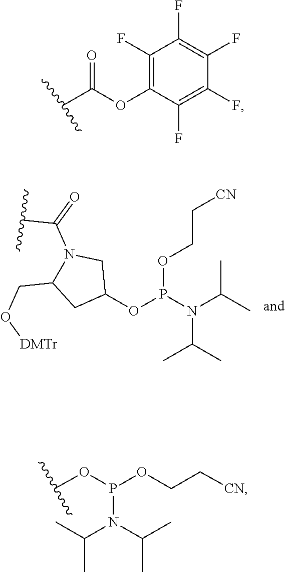
preferably 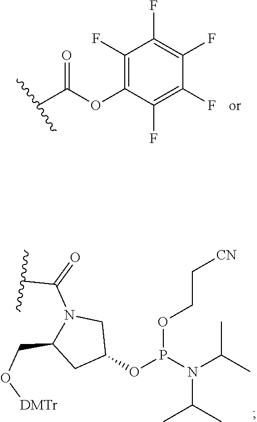
M₃ₐ is chemical bond or —C(O)NH—;
n₁ is independently 4 or 6;
n₂ is independently 0 or 3;
n₃ is 2;
m₁, m₂ and m₃ are all 1;
Link₄ is selected from —(CH₂)₃—, —(CH₂)₁₀—, —(CH₂)₃—C(O)NH—(CH₂)₆— or —(CH₂)₁₀—C(O)NH—(CH₂)₆—, preferably at least one of —(CH₂)₃—, —(CH₂)₁₀— and —(CH₂)₃—C(O)NH—(CH₂)₆—, preferably —(CH₂)₃— or —(CH₂)₁₀—.

26. The formula (I) compound of claim 1, or the pharmaceutical acceptable salt, tautomer or stereoisomer thereof, wherein, the said compound is selected from at least one of the following:

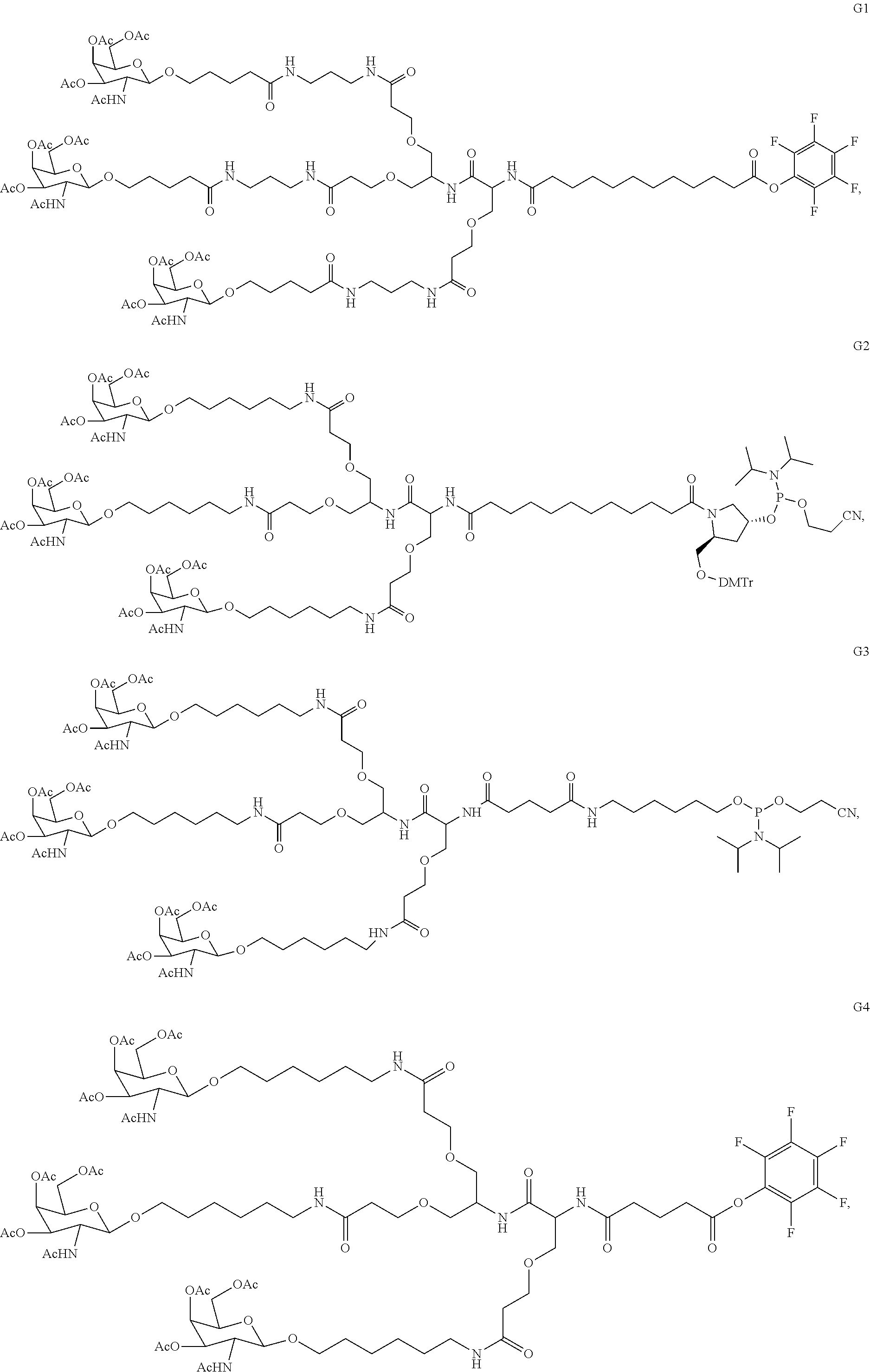

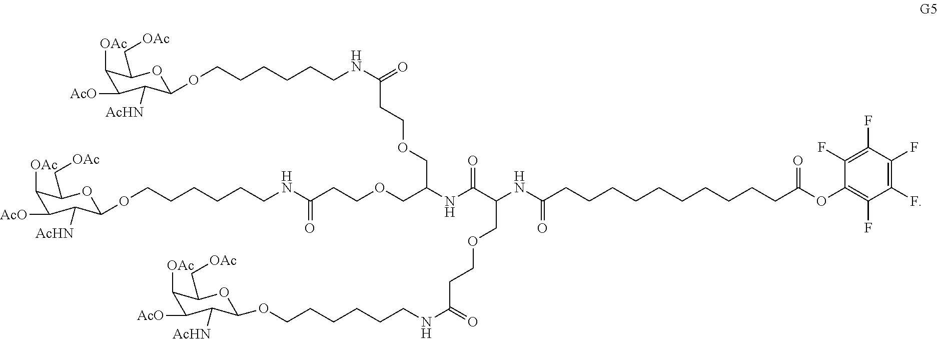 [SMILES: CC(=O)NC1C(OCCCCCCNC(=O)CCOCC(COCCC(=O)NCCCCCCOC2OC(COC(C)=O)C(OC(C)=O)C(OC(C)=O)C2NC(C)=O)NC(=O)C(COCCC(=O)NCCCCCCOC2OC(COC(C)=O)C(OC(C)=O)C(OC(C)=O)C2NC(C)=O)NC(=O)CCCCCCCCCCC(=O)Oc2c(F)c(F)c(F)c(F)c2F)OC(COC(C)=O)C(OC(C)=O)C1OC(C)=O]

27. A conjugate, which has the structure as shown in formula (III): 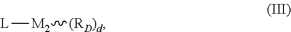
wherein,
L has structure as shown in formula (I′) or formula (II′): 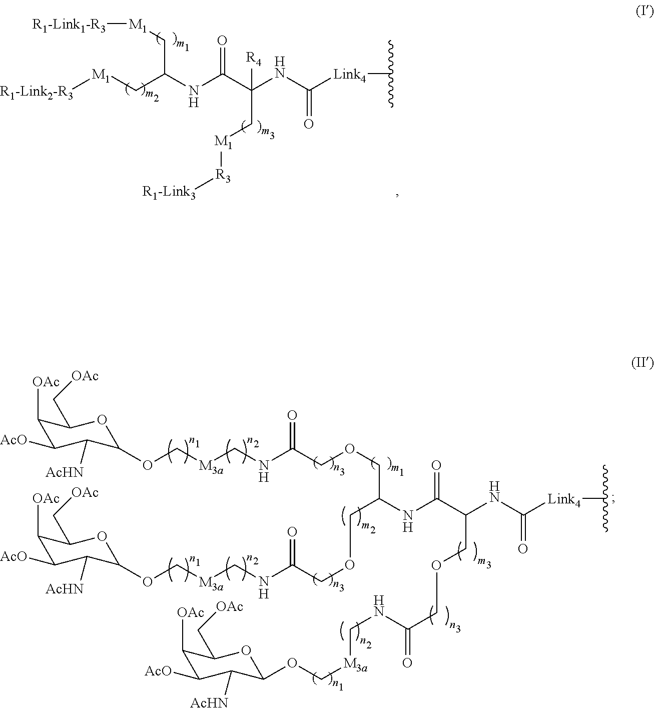 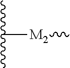
is selected from at least one of 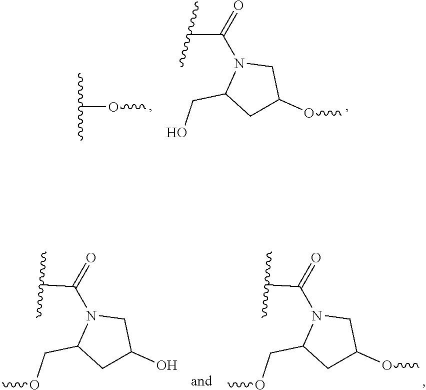
preferably selected from at least one of 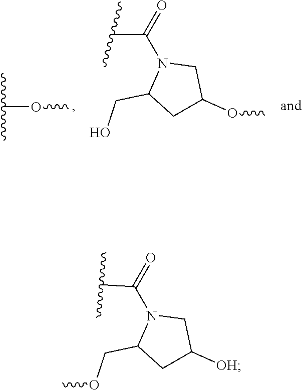
or preferably the 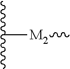
is selected from at least one of 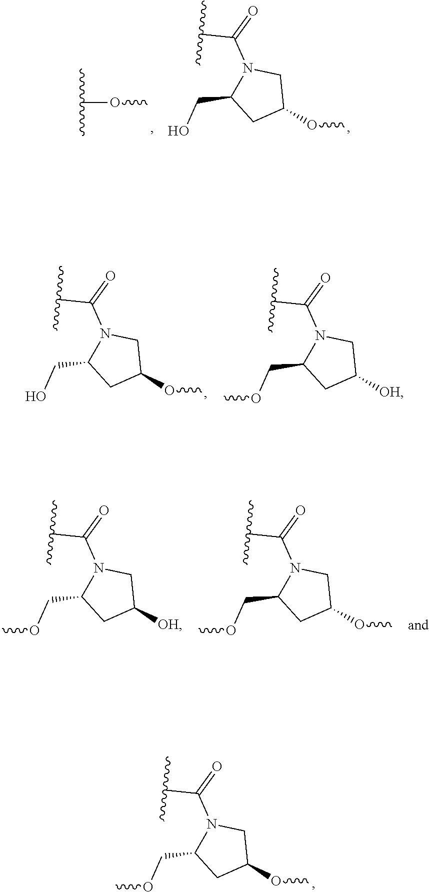
preferably selected from at least one of 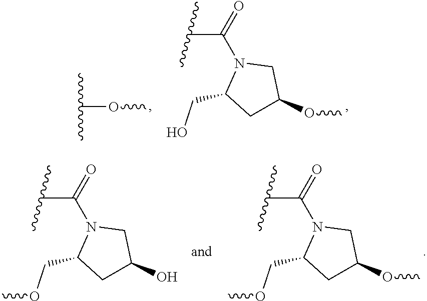
preferably selected from at least one of 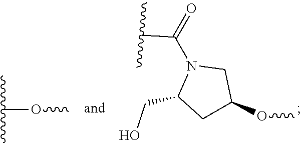
R_{D} is pharmaceutical molecule, the said pharmaceutical molecule is independently selected from at least one of cytotoxic agents, chemotherapeutic agents, somatostatin, toxins, radioisotopes and oligonucleotide strand;
d is 1 or 2, preferably 1;
each remaining groups are as defined in claim 1;
preferably, Link₄ is selected from C₅₋₂₅ linear chain alkylene, preferably C₅₋₂₀ linear chain alkylene, preferably C₈₋₂₀ linear chain alkylene, preferably C₁₀₋₁₇ linear chain alkylene;
preferably, one or multiple methylenes of Link₄, preferably 1, 2 or 3 methylenes, preferably 1 methylene is optionally and independently substituted by R*, R* is independently selected from at least one of —C(O)NH—, —NHC(O)—, —C(O)O— and —OC(O)—, preferably at least one of —C(O)NH— and —C(O)O—, or preferably at least one of —C(O)NH— and —NHC(O)—, preferably —C(O)NH—;
preferably, R* is separated from the carbonyl on the left side of Link₄ by 1-15 methylenes, preferably 1-12 methylenes, preferably 1-10 methylenes, preferably 3-10 methylenes;
R* is separated from R₂ by 1-12 methylenes, preferably 1-10 methylenes, preferably 1-6 methylenes;
preferably, Link₄ is selected from at least one of —(CH₂)₁₀—, —(CH₂)₃—C(O)NH—(CH₂)₆— and —(CH₂)₁₀—C(O)NH—(CH₂)₆—, preferably —(CH₂)₁₀— or —(CH₂)₁₀—C(O)NH—(CH₂)₆—.

28. The formula (III) conjugate of claim 27, or the pharmaceutical acceptable salt, tautomer or stereoisomer thereof, wherein, R_{D} is oligonucleotide strand, optionally, the said oligonucleotide strand is single strand oligonucleotide or double strand oligonucleotide or composition thereof;
preferably, M₂ links to 5′ end, 3′ end or any one nucleotide between 5′ end and 3′ end of at least one strand of the said oligonucleotide by phosphate ester bond, preferably links to 5′ end or 3′ end of at least one strand, preferably links to 5′ end of at least one strand, preferably links to 3′ end of at least one strand;
preferably, M₂ links to sense strand of the said oligonucleotide by phosphate ester bond;
preferably, the said phosphate ester bond is phosphodiester bond or modified phosphate ester bond, preferably phosphodiester bond; preferably, the said phosphate ester bond is selected from thio-modified phosphate ester bond and/or amino-modified phosphate ester bond, preferably thio-modified phosphate ester bond;
preferably, the said 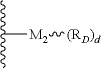
is selected from at least one of the following structure: 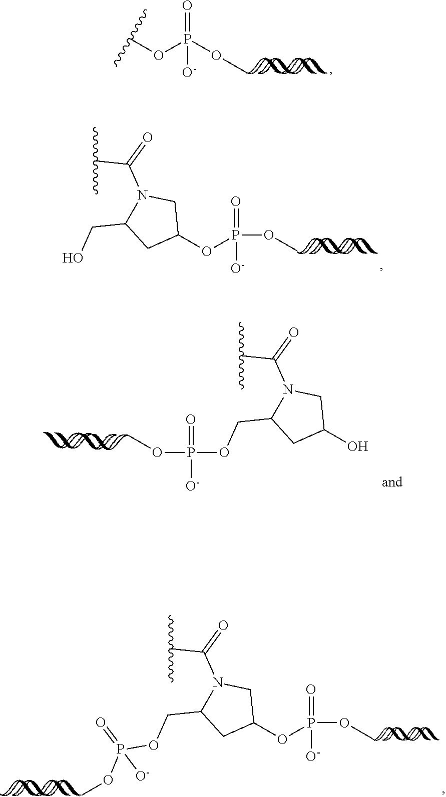
preferably selected from at least one of 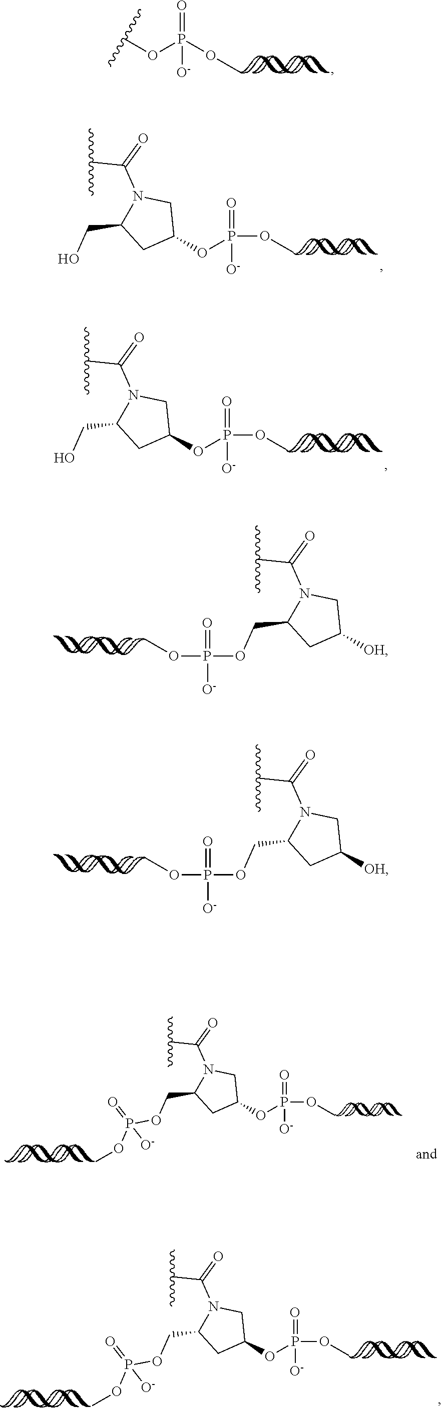
preferably selected from at least one of 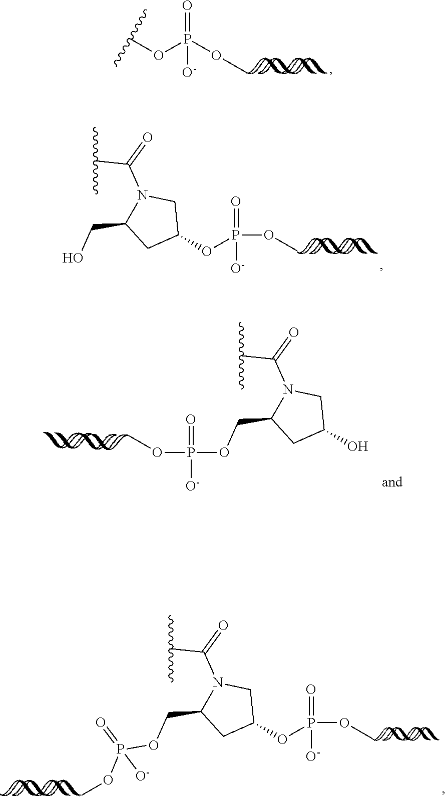
wherein [symbol] is oligonucleotide strand,
and/or the said oligonucleotide strand includes unmodified nucleotide and/or modified nucleotide;
preferably, the said modified nucleotide is respectively and independently selected from at least one of 2′-methoxyethyl modified nucleotide, 2′-O-alkyl modified nucleotide, 2′-O-allyl modified nucleotide, 2′-C-allyl modified nucleotide, 2′-fluoro modified nucleotide, 2′-deoxy modified nucleotide, 2′-hydroxy modified nucleotide, thiophosphoryl backbone modified nucleotide, locked nucleotide modified nucleotide, glycol nucleic acid (GNA) modified nucleotide and unlocked nucleic acid (UNA) modified nucleotide, preferably selected from at least one of 2′-O-alkyl modified nucleotide, 2′-fluoro modified nucleotide, 2′-deoxy modified nucleotide and thiophosphoryl backbone modified nucleotide;
preferably, the said 2′-O-alkyl is 2′-O—C₁₋₆ alkyl, preferably 2′-O-methyl;
preferably, the length of the said oligonucleotide strand is 5-100 bp;
and/or the said oligonucleotide strand is siRNA, ASO or microRNA, preferably siRNA.

29. (canceled)

30. (canceled)

31. The formula (III) conjugate of claim 27, or the pharmaceutical acceptable salt, tautomer or stereoisomer thereof, wherein, the said oligonucleotide strand has terminal modifier, the said terminal modifier is selected from cholesterol, polyethylene glycol, fluorescent probe, biotin, polypeptide, vitamin, or tissue targeting molecule, or the composition thereof;
and/or the ribose-phosphate backbone of the said oligonucleotide strand is substituted by polypeptide nucleic acid (PNA) or morpholine ring antisense nucleotide (PMO);
and/or L is selected from at least one of the following structure: 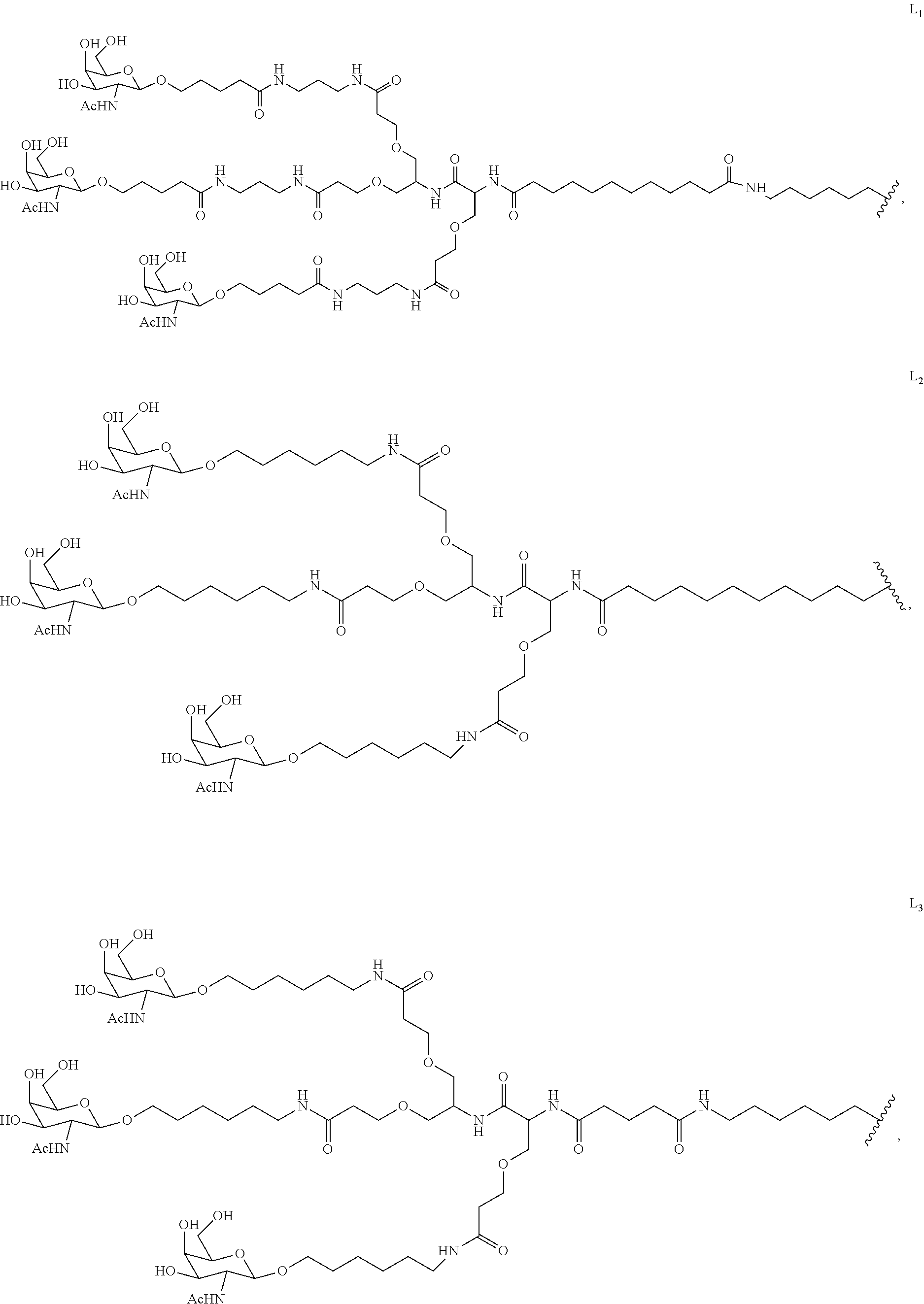 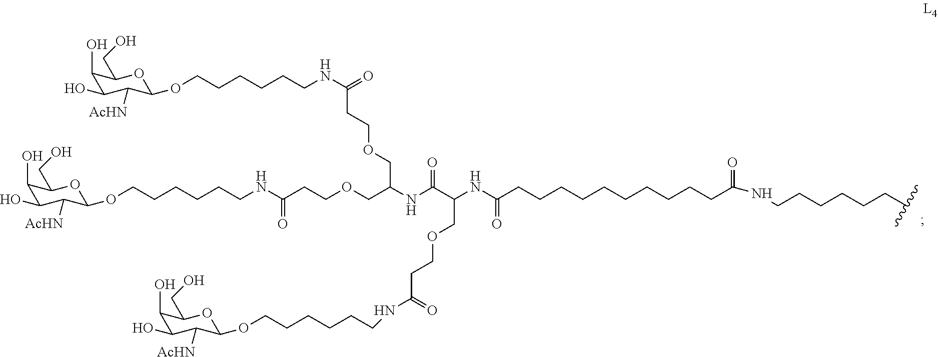 [SMILES: *CCCCCCNC(=O)CCCCCCCCCCC(=O)NC(COCCC(=O)NCCCCCCOC1OC(CO)C(O)C(O)C1NC(C)=O)C(=O)NC(COCCC(=O)NCCCCCCOC1OC(CO)C(O)C(O)C1NC(C)=O)COCCC(=O)NCCCCCCOC1OC(CO)C(O)C(O)C1NC(C)=O]
preferably at least one of the following structure:

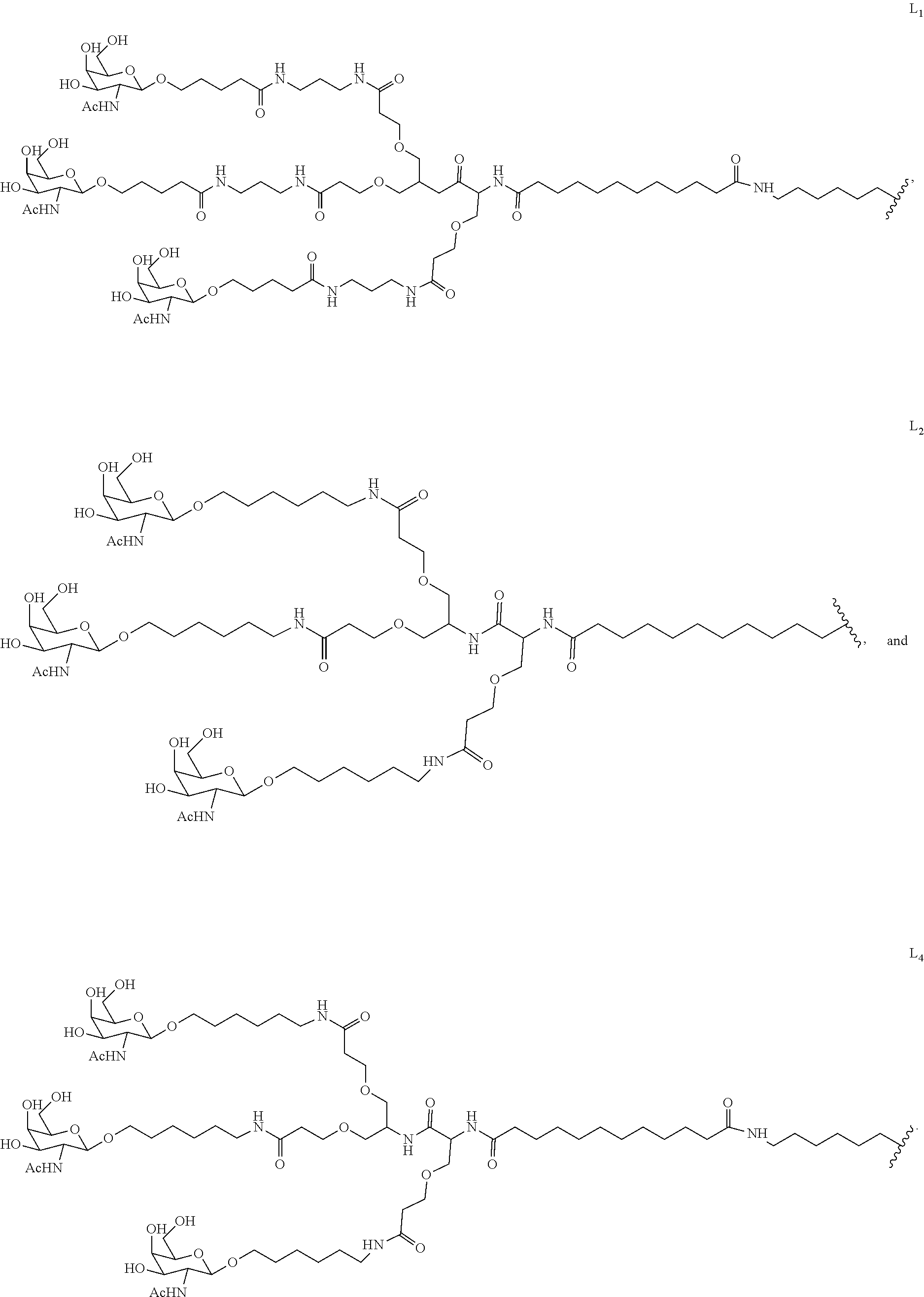

32. (canceled)

33. (canceled)

34. The formula (III) conjugate of claim 28, or the pharmaceutical acceptable salt, tautomer or stereoisomer thereof, wherein, the said conjugate is selected from at least one of the following:

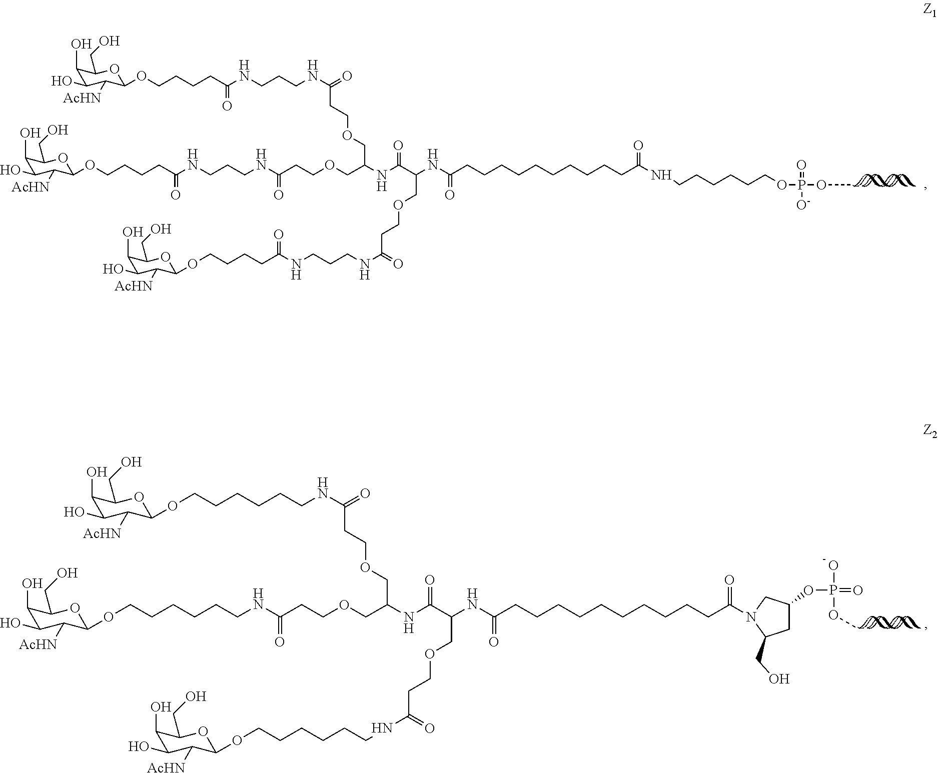

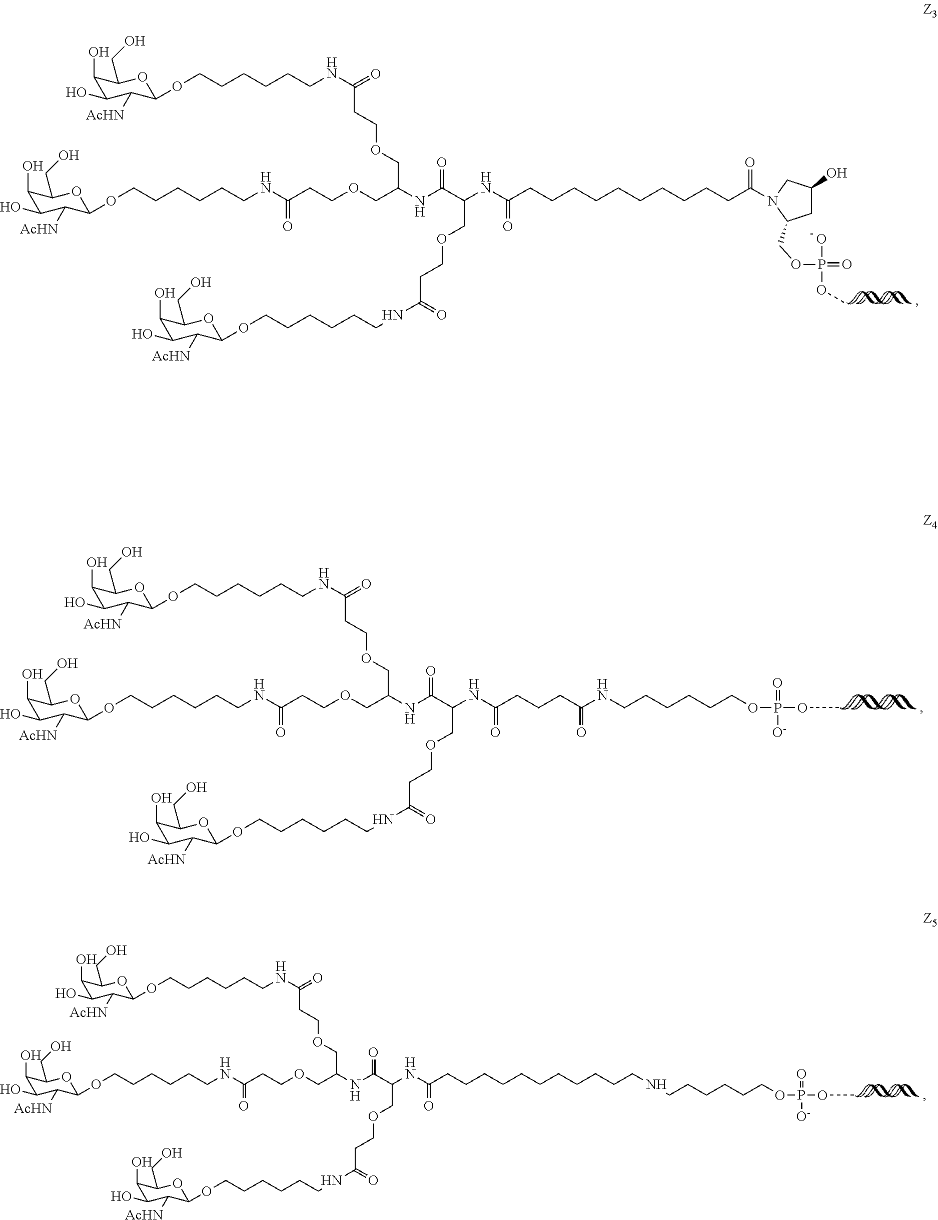

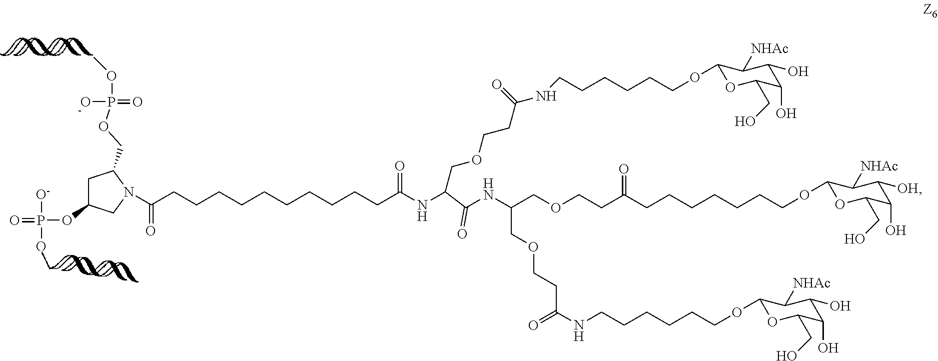 [SMILES: COP(=O)(O)OC[C@H]1C[C@H](OP(=O)([O-])O)CN1C(=O)CCCCCCCCCCC(=O)NC(COCCC(=O)NCCCCCCOC1OC(CO)C(O)C(O)C1NC(C)=O)C(=O)NC(COCCC(=O)CCCCCCCOC1OC(CO)C(O)C(O)C1NC(C)=O)COCCC(=O)NCCCCCCOC1OC(CO)C(O)C(O)C1NC(C)=O]

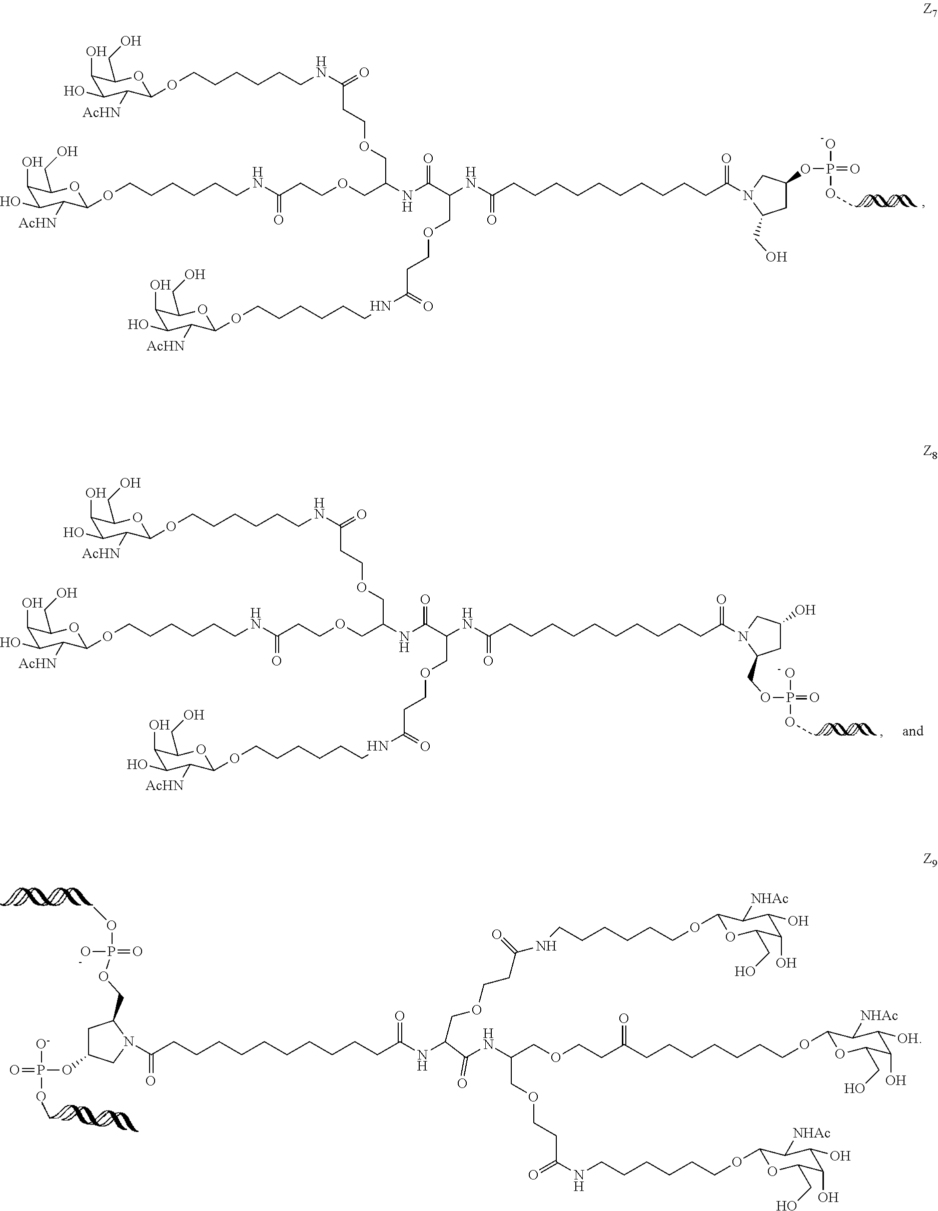

35. A pharmaceutical composition, which includes conjugate of claim 27, and optional selected pharmaceutical acceptable carrier, excipient, adjuvant or vehicle, and optionally other therapeutic agents.

36. (canceled)

37. A method of curing or preventing the disease related to expression or over expression of gene in hepatocyte in subject, including administrating conjugate of claim of 27.

38. (canceled)

39. The method of claim 37, wherein the said gene is selected from HBV gene group, HCV gene group, PCSK9, xanthine oxidase, URAT1, APOB, hepatic fibrosis related gene (AP3S2, AQP2, AZINI, DEGSI, STXBP5L, TLR4, TRPM5), non-alcoholic fatty liver disease related gene (PNPLA3, FDFTl), or primary biliary cirrhosis (HLA-DQB1, IL-12, IL-12RB2), or the combination thereof;
and/or the said disease is selected from at least one of hereditary angioedema, familial tyrosinemia type I or type II, Alagille syndrome, α-1-antitrypsindeficiency, bile acid synthesis and metabolism defect, biliary atresia, cystic fibrosis liver disease, idiopathic neonatal hepatitis, mitochondrial liver disease, progressive familial intrahepatic cholestasis, primary sclerosing cholangitis, transthyretin amyloidosis, hemophilia, homozygous familial hypercholesterolemia, hyperlipidemia, hepatitis B virus (HBV), hepatitis C virus (HCV), steatohepatitis, nonalcoholic steatohepatitis (NASH), non-alcoholic fatty liver disease (NAFLD), hyperglycemia or disease of abnormally increased hepatic glucose production similar to type I and II diabetes, hepatitis and hepatoporphyrin.

40. (canceled)

41. (canceled)

42. (canceled)

43. (canceled)

44. (canceled)

45. A method of curing or preventing the disease related to expression or over expression of gene in hepatocyte in subject, including administrating pharmaceutical composition of claim 35 to subject.

46. The method of claim 45, wherein the said gene is selected from HBV gene group, HCV gene group, PCSK9, xanthine oxidase, URAT1, APOB, hepatic fibrosis related gene (AP3S2, AQP2, AZINI, DEGSI, STXBP5L, TLR4, TRPM5), non-alcoholic fatty liver disease related gene (PNPLA3, FDFTl), or primary biliary cirrhosis (HLA-DQB1, IL-12, IL-12RB2), or the combination thereof;
and/or the said disease is selected from at least one of hereditary angioedema, familial tyrosinemia type I or type II, Alagille syndrome, α-1-antitrypsindeficiency, bile acid synthesis and metabolism defect, biliary atresia, cystic fibrosis liver disease, idiopathic neonatal hepatitis, mitochondrial liver disease, progressive familial intrahepatic cholestasis, primary sclerosing cholangitis, transthyretin amyloidosis, hemophilia, homozygous familial hypercholesterolemia, hyperlipidemia, hepatitis B virus (HBV), hepatitis C virus (HCV), steatohepatitis, nonalcoholic steatohepatitis (NASH), non-alcoholic fatty liver disease (NAFLD), hyperglycemia or disease of abnormally increased hepatic glucose production similar to type I and II diabetes, hepatitis and hepatoporphyrin.


SEQUENCE LISTING (8 sequences)

SEQ ID NO: 1 - RNA, 19 nt, synthetic construct
  source 1..19: mol_type = other RNA
  modified_base join(1,5,8,12,16): mod_base = OTHER; note = deoxyribonucleotide modification
  modified_base 3: mod_base = OTHER; note = thiophosphorylated skeleton modification and deoxyribonucleotide modification
  modified_base join(4,6,7,9,10,11,13,14,15,17): mod_base = OTHER; note = 2'-fluoro modification
  modified_base join(2,18,19): mod_base = OTHER; note = thiophosphorylated skeleton modification and 2'-fluoro modification
gtatgttgcccgtttgtcc

SEQ ID NO: 2 - RNA, 21 nt, synthetic construct
  source 1..21: mol_type = other RNA
  modified_base join(1,5,7,9,11,13,15,17): mod_base = OTHER; note = 2'-O-methyl modification
  modified_base join(3,19,20,21): mod_base = OTHER; note = thiophosphorylated skeleton modification and 2'-O-methyl modification
  modified_base join(2,4): mod_base = OTHER; note = thiophosphorylated skeleton modification and 2'-fluoro modification
  modified_base join(6,8,10,12,14,16,18): mod_base = OTHER; note = 2'-fluoro modification
ggacaaacgggcaacatacct

SEQ ID NO: 3 - RNA, 19 nt, synthetic construct
  source 1..19: mol_type = other RNA
  modified_base join(1,4,5,6,10,11,12,13,14,15,16): mod_base = OTHER; note = 2'-O-methyl modification
  modified_base join(2,3,17,18,19): mod_base = OTHER; note = thiophosphorylated skeleton modification and 2'-O-methyl modification
  modified_base join(7,8,9): mod_base = OTHER; note = 2'-fluoro modification
ggtctggaatgcaaagtca

SEQ ID NO: 4 - RNA, 19 nt, synthetic construct
  source 1..19: mol_type = other RNA
  modified_base join(1,5,7,9,11,13,15): mod_base = OTHER; note = 2'-O-methyl modification
  modified_base join(3,17,18,19): mod_base = OTHER; note = thiophosphorylated skeleton modification and 2'-O-methyl modification
  modified_base 2: mod_base = OTHER; note = thiophosphorylated skeleton modification and 2'-fluoro modification
  modified_base join(4,6,8,10,12,14,16): mod_base = OTHER; note = 2'-fluoro modification
tgactttgcattccagacc

SEQ ID NO: 5 - DNA, 23 bp, synthetic construct
  source 1..23: mol_type = other DNA; note = Forward primer sequence of actin gene
tcaagatcattgctcctcctgag

SEQ ID NO: 6 - DNA, 21 bp, synthetic construct
  source 1..21: mol_type = other DNA; note = Reverse primer sequence of actin gene
acatctgctggaaggtggaca

SEQ ID NO: 7 - DNA, 23 bp, synthetic construct
  source 1..23: mol_type = other DNA; note = Forward primer sequence of human PCSK9 gene
aagccaagcctcttcttacttca

SEQ ID NO: 8 - DNA, 21 bp, synthetic construct
  source 1..21: mol_type = other DNA; note = Reverse primer sequence of human PCSK9 gene
cctgggtgataacggaaaaag